(19) United States
(12) Patent Application Publication
(10) Pub. No.: US 2026/0364183 A1
(43) Pub. Date: Nov. 19, 2026

(54) HYDROXYLAMINE-HYDROXYPIPERIDINE CONJUGATE COMPOUNDS, COMPOSITIONS, AND PROCESSES FOR MAKING AND USING THE SAME

(71) Applicant: Zevra Therapeutics, Inc., Celebration, FL (US)

(72) Inventor: Sven Guenther, Coralville, IA (US)

(21) Appl. No.: 19/430,100

(22) Filed: Dec. 22, 2025

Related U.S. Application Data
(60) Provisional application No. 63/738,273, filed on Dec. 23, 2024.

(51) Int. Cl. A61K 47/55; A61K 47/54

(52) U.S. Cl. CPC A61K 47/55; A61K 47/545

(57) ABSTRACT

The present technology relates to a genus of compounds comprising a subunit of arimoclomol covalently linked to a subunit of miglustat. The present technology further relates to compositions comprising said compounds and method of treating neurological diseases and disorders using said compounds.

### STATEMENT OF RELATED APPLICATIONS

This application claims priority to U.S. Application No. 63/738,273, filed on Dec. 23, 2024, the entire contents of which are incorporated herein by reference.

### STATEMENT OF FEDERAL FUNDING

No U.S. Federal Funding was used to develop the instant technology.

### BACKGROUND OF THE TECHNOLOGY

The lysosomal storage diseases are a rare group of diseases, characterized by the accumulation of substances in the lysosomal compartment and resulting destabilization hereof, with a resulting devastating effect for affected individuals. Substances accumulate in the lysosomal compartment due to deficiencies in the enzymes involved in their catabolism. Among these lysosomal storage disorders are Niemann-Pick type C (NPC) disease and Gaucher disease (GD), both of which are caused by a build-up of fatty substances.

Currently, arimoclomol citrate is approved in the United States as an oral medicament for treating NPC and GD, respectively. Miglustat is approved in the European Union as an oral medicament for treating Gaucher disease. Both arimoclomol and miglustat however need to be administered multiple times a day in order to maintain therapeutically effective plasma levels.

In addition, it is believed in the art that brain absorption of miglustat is low, likely around 20%, which results in requiring a higher dosage to achieve therapeutic effect before the remaining miglustat is cleared from the blood by the kidneys. Moreover, miglustat often produces gastrointestinal side effects that are believed to be caused by inhibition of intestinal disaccharidases resulting in, for example, carbohydrate malabsorption, diarrhea, abdominal pain, flatulence, bloating, vomiting, nausea, anorexia, dyspepsia, nutrient deficiency, weight loss, and stunted growth.

Therefore, there further exists a need in the art for forms of arimoclomol that can provide therapeutic effect with 1 or 2 oral doses per day. It is believed in the art that brain absorption of miglustat is low, likely around 20%, which results in requiring a higher dosage to achieve therapeutic effect; and that the higher dosage requirement of miglustat results in gastrointestinal effects believed to be caused by inhibition of intestinal disaccharidases, which causes for example, carbohydrate malabsorption, diarrhea, abdominal pain, flatulence, bloating, vomiting, nausea, anorexia, dyspepsia, nutrient deficiency, weight loss, and stunted growth.

Additionally, there exists a need in the art for forms of miglustat with increased bioavailability and more consistent plasma levels.

There exists a further need in the art for forms of miglustat that can more efficiently cross the blood-brain barrier and result in higher brain absorption and reduced side effects. There exists a still further need in the art for forms of miglustat that reduce the number of daily unit doses administered from 3 down to 1 or 2.

Additionally, there exists a need in the art for methods of treating lysosomal storage disorders using prodrugs of arimoclomol and miglustat that provide improved bioavailability and/or more consistent plasma levels of the active components over longer periods of time. Currently, oral treatments for lysosomal storage disorders are accomplished by multiple doses of drugs such as arimoclomol and/or salts thereof.

Finally, there exists a need in the art for forms of arimoclomol with slower metabolisms that result in longer release profiles that provide more consistent plasma levels of the drugs.

### SUMMARY OF THE DISCLOSURE

In certain aspects, the present technology relates to a compound of Formula I:

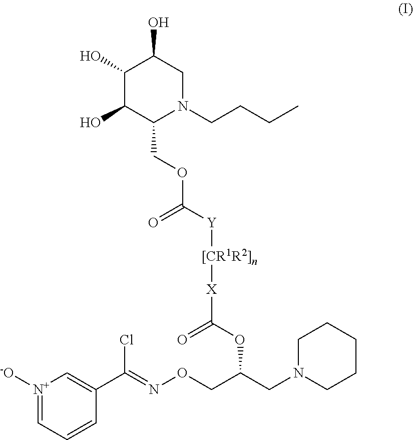

wherein X is selected from the group consisting of O, N, or absent,
Y is selected from the group consisting of O, N, or absent,
n is 1-10, and
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to a compound of Formula I, wherein n is 1-3, alternatively 4-7, alternatively 8-10, alternatively 1-5, alternatively 3-7, alternatively 6-10.

In a further aspect, the present technology relates to a compound of Formula I, wherein X and Y are absent; or a pharmaceutically acceptable salt thereof.

In a further aspect, the present technology relates to a compound of Formula I, wherein the compound of Formula I is selected from the group consisting of:

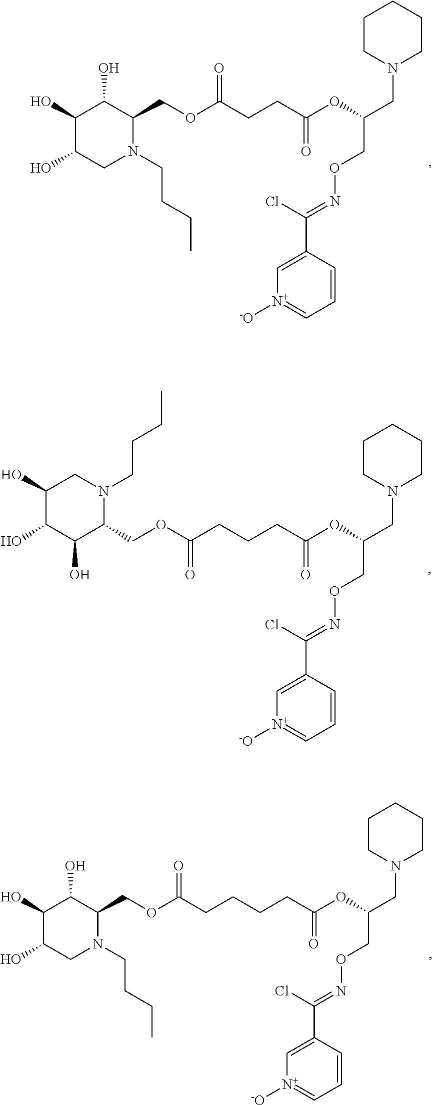

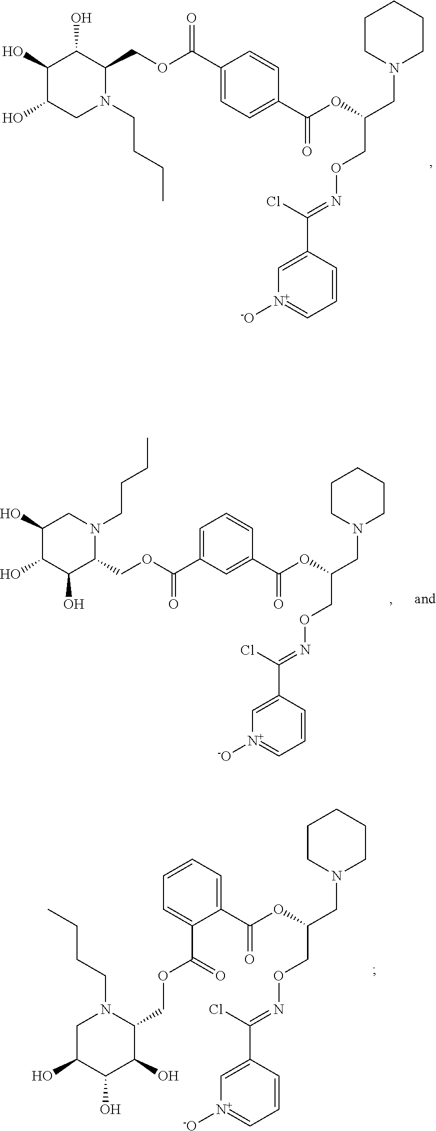

or a pharmaceutically acceptable salt thereof.

In a further aspect, the present technology relates to a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further aspect, the present technology relates to a compound of Formula I, wherein one of X or Y is absent; or a pharmaceutically acceptable salt thereof. In a further aspect, the compound of Formula I is selected from the group consisting of:

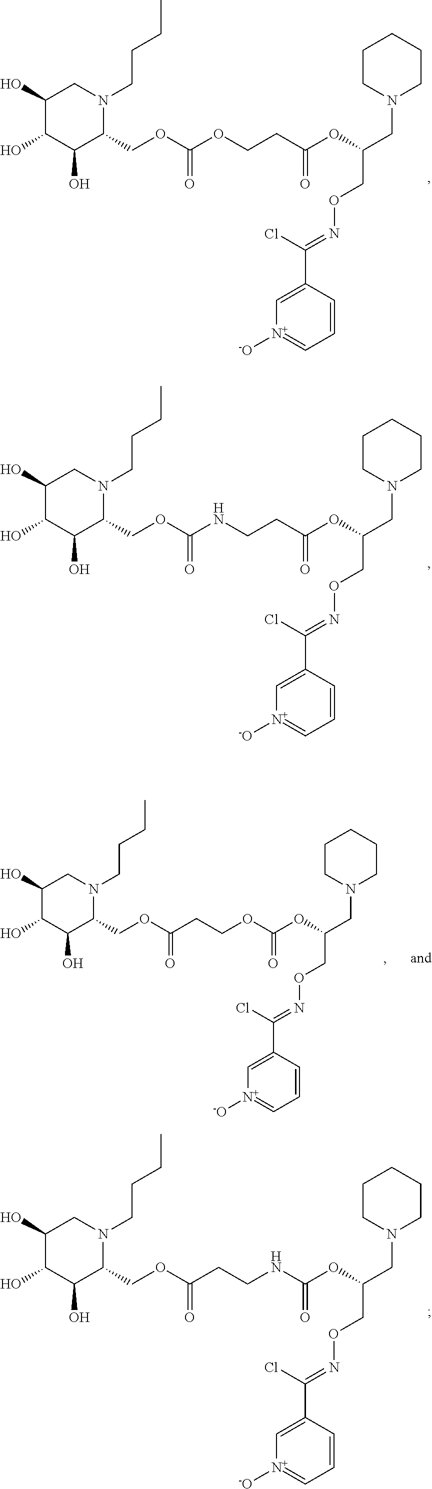

or a pharmaceutically acceptable salt thereof.

In a further aspect, the present technology relates to a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further aspect, the present technology relates to a compound of Formula I, wherein X is N or O, and Y is N or O; or a pharmaceutically acceptable salt thereof. In a further aspect, the compound of Formula I is selected from the group consisting of:

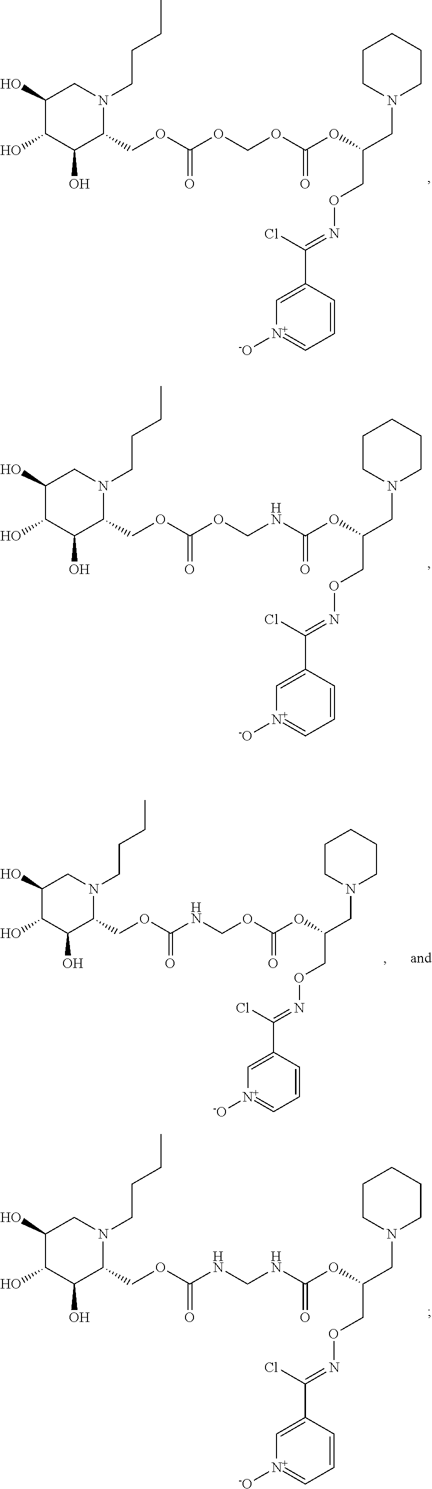

or a pharmaceutically acceptable salt thereof.

In a further aspect, the present technology relates to a compound of Formula I, wherein the compound of Formula I is present in a form selected from the group consisting of a stereoisomer and a racemate, or a mixture thereof.

In a further aspect, the present technology relates to a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a compound of Formula II:

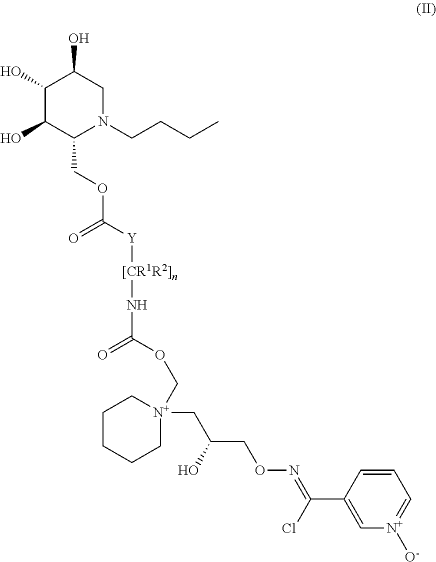

wherein Y is selected from the group consisting of O, N, or absent,
n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to a compound of Formula II, wherein n is 1-3, alternatively 4-7, alternatively 8-10, alternatively 1-5, alternatively 3-7, alternatively 6-10.

In a further aspect, the present technology relates to a compound of Formula II, wherein Y is absent; or a pharmaceutically acceptable salt thereof. In a still further aspect, the compound of Formula II is selected from the group consisting of:

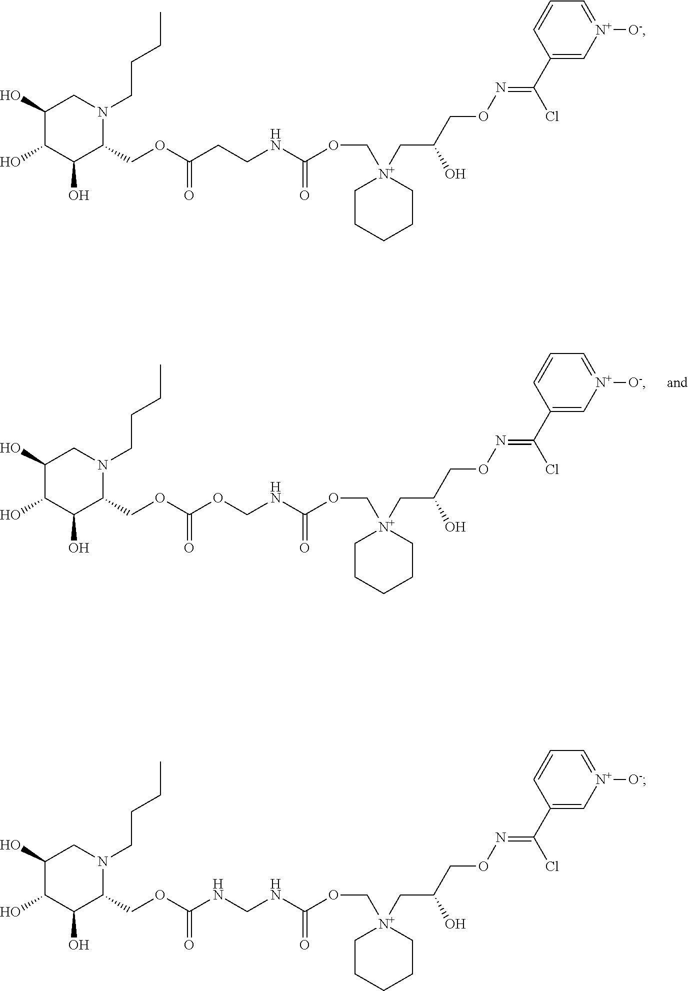

or a pharmaceutically acceptable salt thereof.

In a further aspect, the present technology relates to a compound of Formula II, wherein the compound of Formula II is present in a form selected from the group consisting of a stereoisomer and a racemate, or a mixture thereof.

In a further aspect, the present technology relates to a compound of Formula II, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a compound of Formula III:

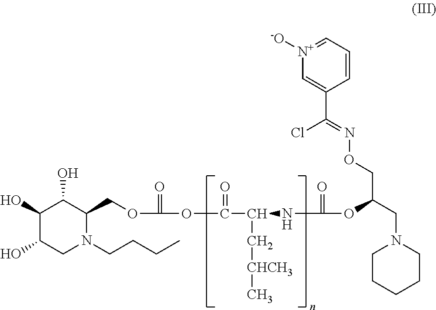

wherein n is 1-50,
or a pharmaceutically acceptable salt thereof.

In a further aspect, the present technology relates to a compound of Formula III, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a compound of Formula III, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a compound of Formula IV:

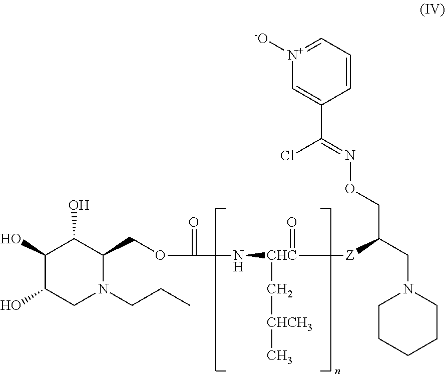

wherein Z is selected from the group consisting of O or OC(═O)O,
n is 1-50,
or a pharmaceutically acceptable salt thereof.

In a further aspect, the present technology relates to a compound of Formula IV, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a compound of Formula IV, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a compound of Formula V:

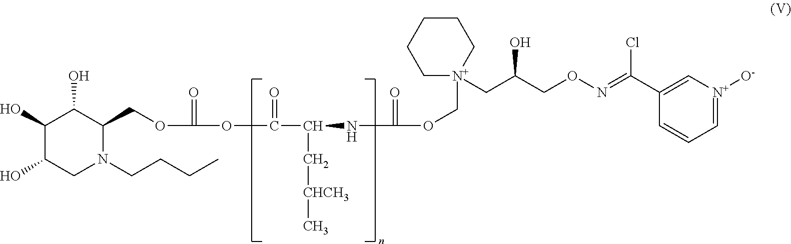

wherein n is 1-50,
   or a pharmaceutically acceptable salt thereof.

In a further aspect, the present technology relates to a compound of Formula V, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a compound of Formula V, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a composition comprising:
a therapeutically effective amount of a compound of Formula I:

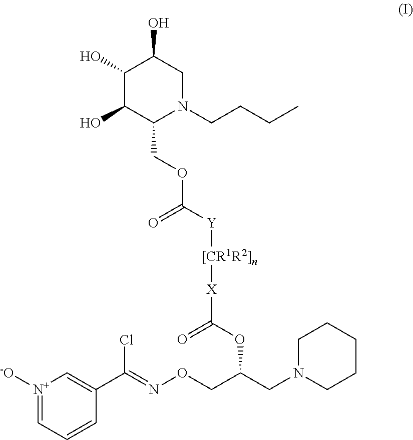

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkyl carb onyl amino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, aryl carb onyl amino, arylcarbonyloxy, arylcycloalkyl, aryloxy, aryloxyalkyl, arylsulfinyl, arylsulfinylalkyl, arylsulfonyl, aryl sulfonyl amino, arylthio, arylthioalkyl, cycloalkenyl, cycloalkenylalkyl, cycloalkyl, cycloalkylalkyl, cycloalkyl amino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroaryl amino, heteroarylcarbonyl, heteroaryl carb onyl amino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a composition comprising the therapeutically effective amount of the compound of Formula I, wherein n is 1-3, alternatively 4-7, alternatively 8-10, alternatively 1-5, alternatively 3-7, alternatively 6-10.

In a further aspect, the present technology relates to a composition comprising the therapeutically effective amount of the compound of Formula I, wherein X and Y are absent; or a pharmaceutically acceptable salt thereof. In a still further aspect, the compound of Formula I, wherein the compound of Formula I is selected from the group consisting of:

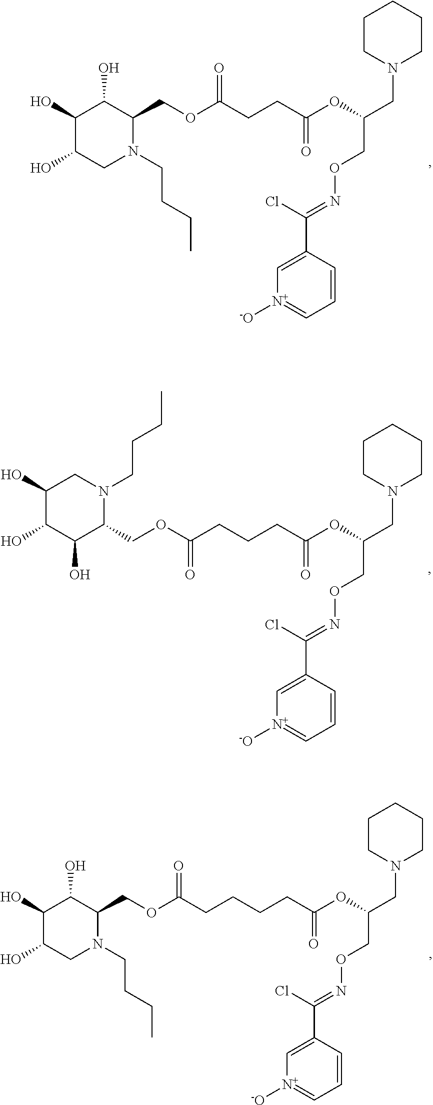

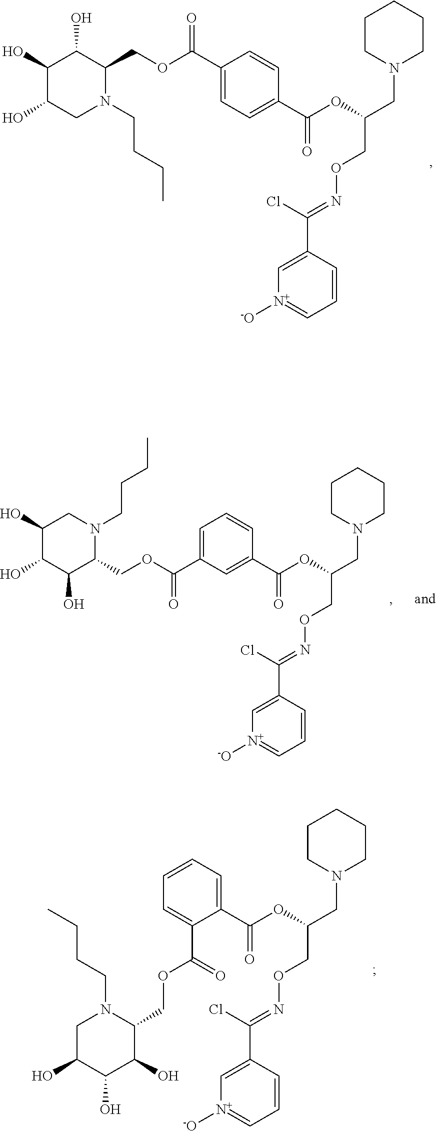

or a pharmaceutically acceptable salt thereof.

In a further aspect, the present technology relates to a composition comprising the therapeutically effective amount of the compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further aspect, the present technology relates to a composition comprising the therapeutically effective amount of the compound of Formula I, wherein X or Y is absent; or a pharmaceutically acceptable salt thereof. In a still further aspect, the compound of Formula I is selected from the group consisting of:

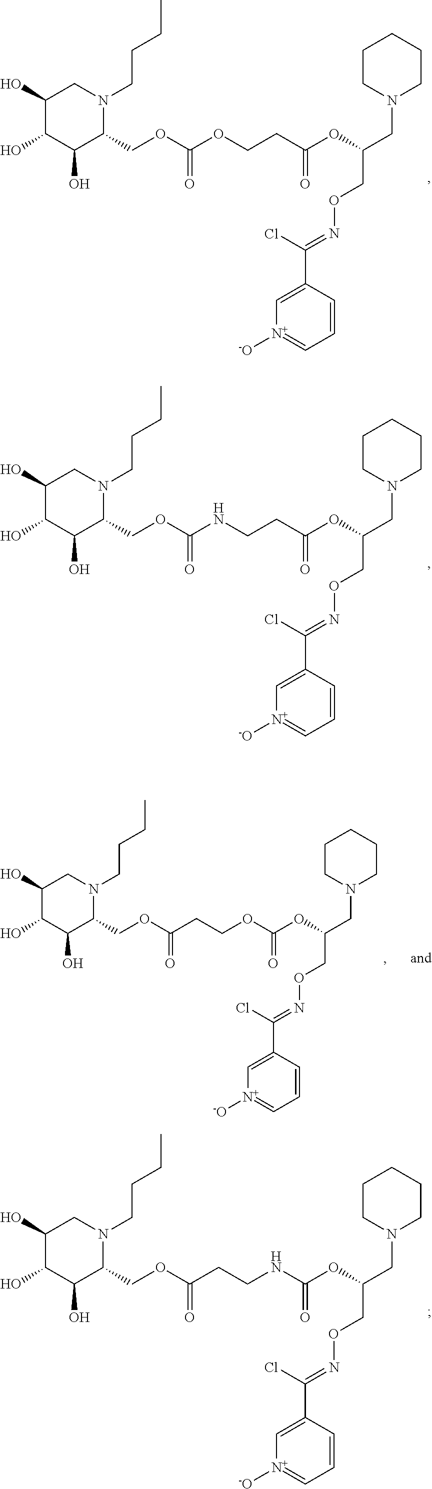

or pharmaceutically acceptable salt thereof.

In a further aspect, the present technology relates to a composition comprising the therapeutically effective amount of the compound of Formula I, wherein the compound of Formula I is present in a form selected from the group consisting of a stereoisomer and a racemate, or a mixture thereof.

In a further aspect, the present technology relates to a composition comprising the therapeutically effective amount of the compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further aspect, the present technology relates to a composition comprising the therapeutically effective amount of the compound of Formula I, wherein X is N or O, and Y is N or O; or a pharmaceutically acceptable salt thereof. In a still further aspect, the compound of Formula I is selected from the group consisting of:

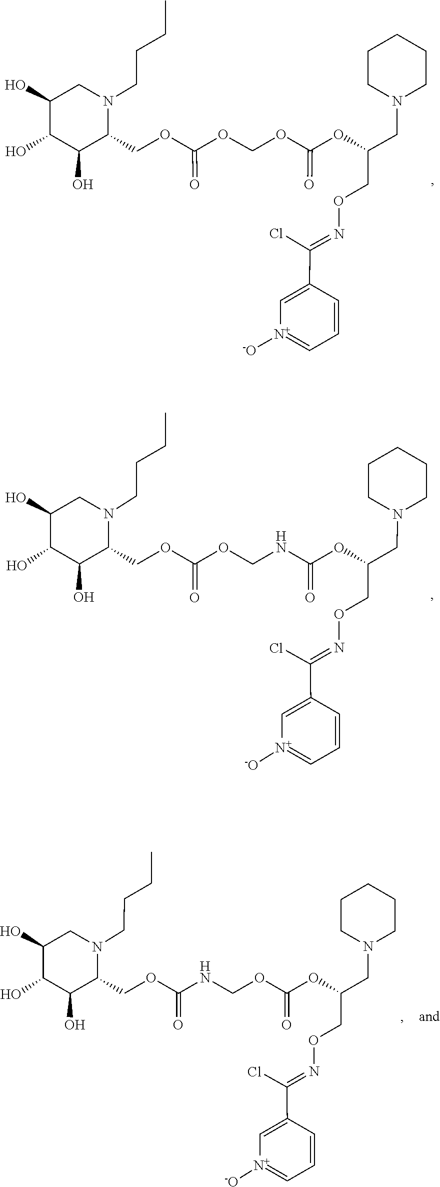

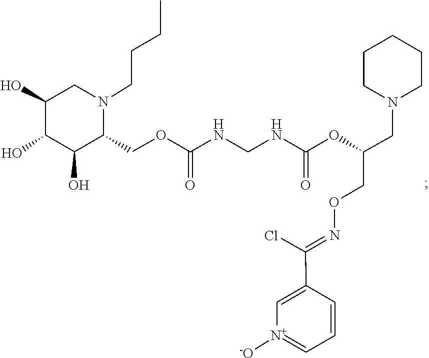

or a pharmaceutically acceptable salt thereof.

In a further aspect, the present technology relates to a composition comprising the therapeutically effective amount of the compound of Formula I, wherein the compound of Formula I is present in a form selected from the group consisting of a stereoisomer and a racemate, or a mixture thereof.

In a further aspect, the present technology relates to a composition comprising the therapeutically effective amount of the compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a composition comprising: a therapeutically effective amount of a compound of Formula II:

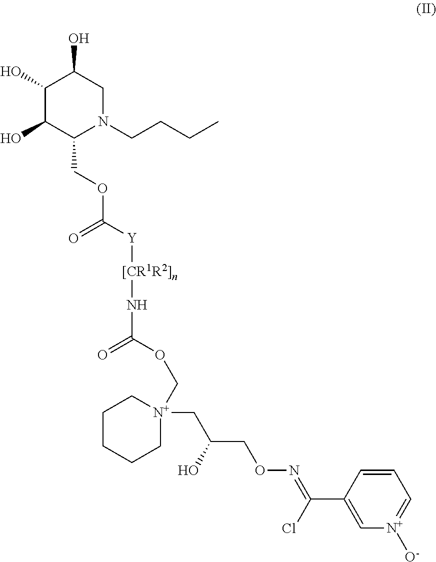

wherein Y is selected from the group consisting of O, N, or absent,
n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to a composition comprising the therapeutically effective amount of the compound of Formula II, wherein n is 1-3, alternatively 4-7, alternatively 8-10, alternatively 1-5, alternatively 3-7, alternatively 6-10.

In a further aspect, the present technology relates to a composition comprising the therapeutically effective amount of the compound of Formula II, wherein Y is absent; or a pharmaceutically acceptable salt thereof. In a still further aspect, the compound of Formula II is selected from the group consisting of:

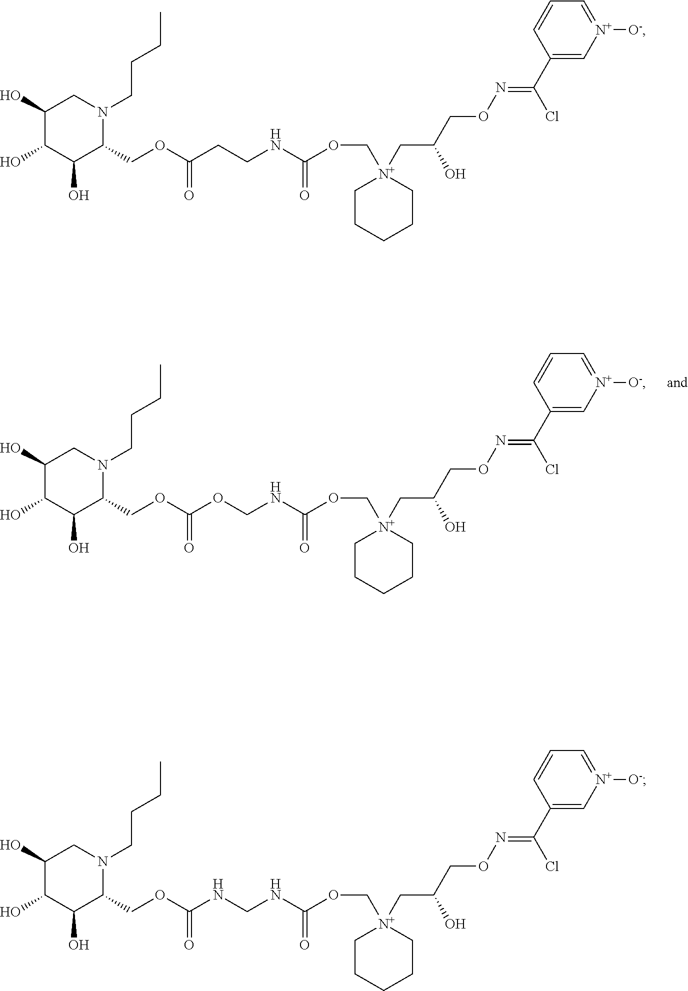

or a pharmaceutically acceptable salt thereof.

In a further aspect, the present technology relates to a composition comprising the therapeutically effective amount of the compound of Formula II, wherein the compound of Formula II is present in a form selected from the group consisting of a stereoisomer and a racemate, or a mixture thereof.

In a further aspect, the present technology relates to a composition comprising the therapeutically effective amount of the compound of Formula II, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspect, the present technology a composition comprising:
a therapeutically effective amount of a compound of Formula III:

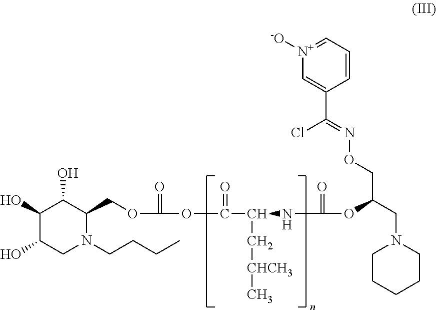

wherein n is 1-50,
or a pharmaceutically acceptable salt thereof.

In a still further aspect, the present technology relates to a composition comprising the therapeutically effective amount of the compound of Formula III, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50

In a further aspect, the present technology relates to a composition comprising the therapeutically effective amount of the compound of Formula III, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a composition comprising:
a therapeutically effective amount of a compound of Formula IV:

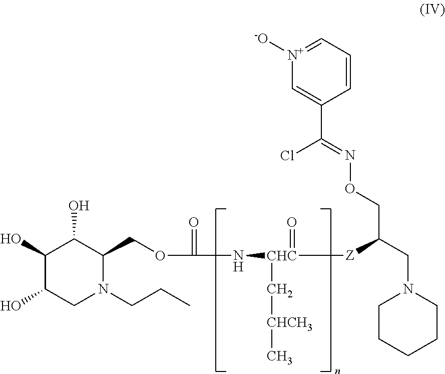

wherein Z is selected from the group consisting of O or OC(═O)O,
n is 1-50,
or a pharmaceutically acceptable salt thereof.

In a still further aspect, the present technology relates to a composition comprising the therapeutically effective amount of the compound of Formula IV, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50

In a further aspect, the present technology relates to a composition comprising the therapeutically effective amount of the compound of Formula IV, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a composition comprising:
a therapeutically effective amount of a compound of Formula V:

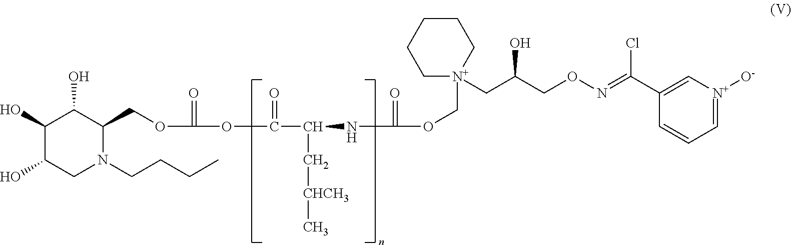

wherein n is 1-50,
or a pharmaceutically acceptable salt thereof.

In a still further aspect, the present technology relates to a composition comprising the therapeutically effective amount of the compound of Formula V, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50

In a further aspect, the present technology relates to a composition comprising the therapeutically effective amount of the compound of Formula V, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further aspect, the present technology relates to a composition comprising the therapeutically effective amount of the compound selected from the group consisting of Formula I to Formula V, wherein the composition further comprises at least one excipient.

In a further aspect, the present technology relates to a composition comprising the therapeutically effective amount of the compound selected from the group consisting of Formula I to Formula V, wherein the at least one excipient is selected from the group consisting of antiadherents, binders, coatings, disintegrants, gel forming agents, fillers, flavors and colors, glidants, lubricants, preservatives, sorbents and sweeteners

In a further aspect, the present technology relates to s composition comprising the therapeutically effective amount of the compound selected from the group consisting of Formula I to Formula V, wherein the composition further comprises at least one additional active pharmaceutical ingredient, wherein the at least one additional active pharmaceutical ingredient is selected from the group consisting of bimoclomol, miglustat, eliglustat, N-acetyl-L-leucine and 2-hydroxypropyl-β-cyclodextrin, arimoclomol, and combinations thereof.

In a further aspect, the present technology relates to a composition comprising the therapeutically effective amount of the compound selected from the group consisting of Formula I to Formula V, wherein the at least one additional active pharmaceutical ingredient is selected from miglustat, N-acetyl-L-leucine, and combinations thereof.

In a further aspect, the present technology relates to a composition comprising the therapeutically effective amount of the compound selected from the group consisting of Formula I to Formula V, wherein the at least one additional active pharmaceutical ingredient is a combination of miglustat and N-acetyl-L-leucine.

In a further aspect, the present technology relates to a composition comprising the therapeutically effective amount of the compound selected from the group consisting of Formula I to Formula V, wherein the composition is formulated for oral administration.

In a further aspect, the present technology relates to a composition comprising the therapeutically effective amount of the compound selected from the group consisting of Formula I to Formula V, wherein the composition is a solid oral dosage formulation.

In a further aspect, the present technology relates to a composition comprising the therapeutically effective amount of the compound selected from the group consisting of Formula I to Formula V, wherein the solid oral dosage formulation is selected from the group consisting of a sublingual, a gummy, a chewable tablet, a rapidly dissolving tablet, a tablet, a capsule, a caplet, a troche, a lozenge, an oral powder, a solution, a thin strip, an oral thin film (OTF), and an oral strip.

In a further aspect, the present technology relates to a composition comprising the therapeutically effective amount of the compound selected from the group consisting of Formula I to Formula V, wherein the composition is a liquid oral dosage formulation.

In a further aspect, the present technology relates to a composition comprising the therapeutically effective amount of the compound selected from the group consisting of Formula I to Formula V, wherein the liquid oral dosage formulation is selected from the group consisting of syrups, emulsions, solutions, and suspensions.

In a further aspect, the present technology relates to a composition comprising the therapeutically effective amount of the compound selected from the group consisting of Formula I to Formula V wherein the therapeutically effective amount of the compound of Formula I ranges from about 1 to about 5000 mg per day.

In a further aspect, the present technology relates to a composition comprising the therapeutically effective amount of the compound selected from the group consisting of Formula I to Formula V, wherein the therapeutically effective amount of the compound of Formula I is administered as a unit dose.

In a further aspect, the present technology relates to a composition comprising the therapeutically effective amount of the compound selected from the group consisting of Formula I to Formula V, wherein the composition is an extended-release formulation, wherein the extended-release formulation further comprises a release controlling agent.

In a further aspect, the present technology relates to a composition comprising the therapeutically effective amount of the compound selected from the group consisting of Formula I to Formula V, wherein the release controlling agent is selected from the group consisting of polymer coating, polymer matrix, and combinations thereof.

In a further aspect, the present technology relates to a composition comprising the therapeutically effective amount of the compound selected from the group consisting of Formula I to Formula V, wherein the release controlling agent is a polymer coating selected from the group consisting of polyethylene oxides (PEO), hydroxypropyl cellulose (HPC), methylcellulose (MC), hydroxypropyl methylcellulose (HPMC), hydroxyethyl cellulose (HEC), ethylcellulose (EC), and sodium carboxymethyl cellulose (CMC)

In a further aspect, the present technology relates to a composition comprising the therapeutically effective amount of the compound selected from the group consisting of Formula I to Formula V, wherein the release controlling agent is a polymer matrix selected from the group consisting of hydroxypropylmethylcellulose (HPMC), ethylcellulose (EC), methylcellulose, hypromellose acetate succinate, hypromellose phthalate, cellulose acetate, glycerin monostearate, glyceryl monooleate, glyceryl palmitate, glyceryl behenate, hydrogenated vegetable oil, guar gum, polyvinyl alcohol, alginates, xanthan gum, carnauba wax, yellow wax, white wax, zein, carrageenan, carbomers and agar.

In a further aspect, the present technology relates to a composition comprising a therapeutically effective amount of the compound selected from the group consisting of Formula I to Formula V, wherein the release controlling agent is a combination of a polymer coating and a polymer matrix.

In certain aspects, the present technology relates to a method of treating a neurodegenerative or metabolic disorder in a patient in need thereof, the method comprising:
administering to the patient in need thereof a composition comprising a therapeutically effective amount of a compound of Formula I:

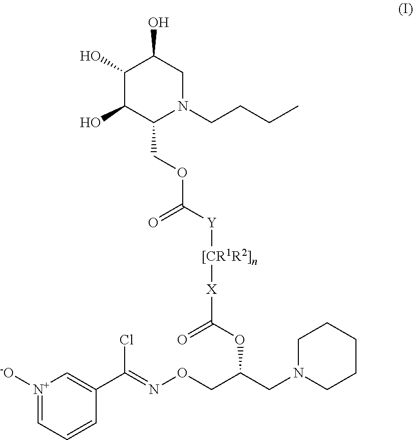

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a method of treating a neurodegenerative or metabolic disorder in a patient in need thereof, the method comprising administering to the patient in need thereof a composition comprising a therapeutically effective amount of a compound of Formula I, wherein n is 1-3, alternatively 4-7, alternatively 8-10, alternatively 1-5, alternatively 3-7, alternatively 6-10

In a further aspect, the present technology relates to a method of treating a neurodegenerative or metabolic disorder in a patient in need thereof, the method comprising administering to the patient in need thereof a composition comprising a therapeutically effective amount of a compound of Formula I, wherein X and Y are absent; or a pharmaceutically acceptable salt thereof.

In a further aspect, the present technology relates to a method of treating a neurodegenerative or metabolic disorder in a patient in need thereof, the method comprising administering to the patient in need thereof a composition comprising a therapeutically effective amount of a compound of Formula I, wherein the compound of Formula I is selected from the group consisting of:

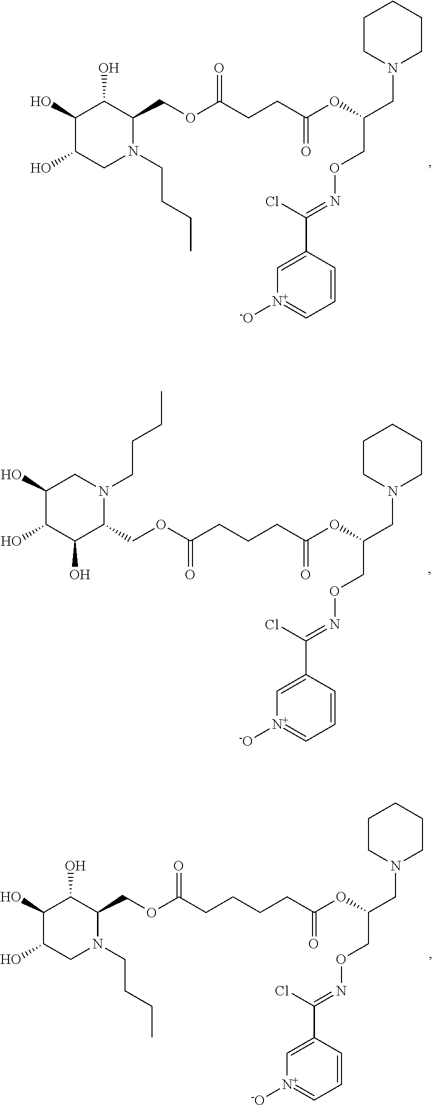

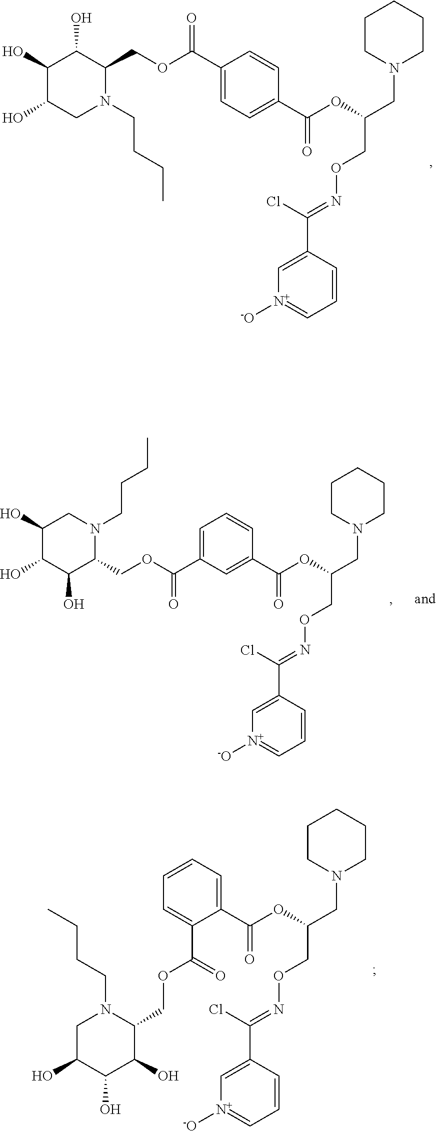

or a pharmaceutically acceptable salt thereof.

In a further aspect, the present technology relates to a method of treating a neurodegenerative or metabolic disorder in a patient in need thereof, the method comprising administering to the patient in need thereof a composition comprising a therapeutically effective amount of a compound of Formula I, wherein the compound of Formula I is present in a form selected from the group consisting of a stereoisomer and a racemate, or a mixture thereof.

In a further aspect, the present technology relates to a method of treating a neurodegenerative or metabolic disorder in a patient in need thereof, the method comprising administering to the patient in need thereof a composition comprising a therapeutically effective amount of a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further aspect, the present technology relates to a method of treating a neurodegenerative or metabolic disorder in a patient in need thereof, the method comprising administering to the patient in need thereof a composition comprising a therapeutically effective amount of a compound of Formula I, wherein X or Y is absent; or a pharmaceutically acceptable salt thereof. In a further aspect, the compound of Formula I is selected from the group consisting of:

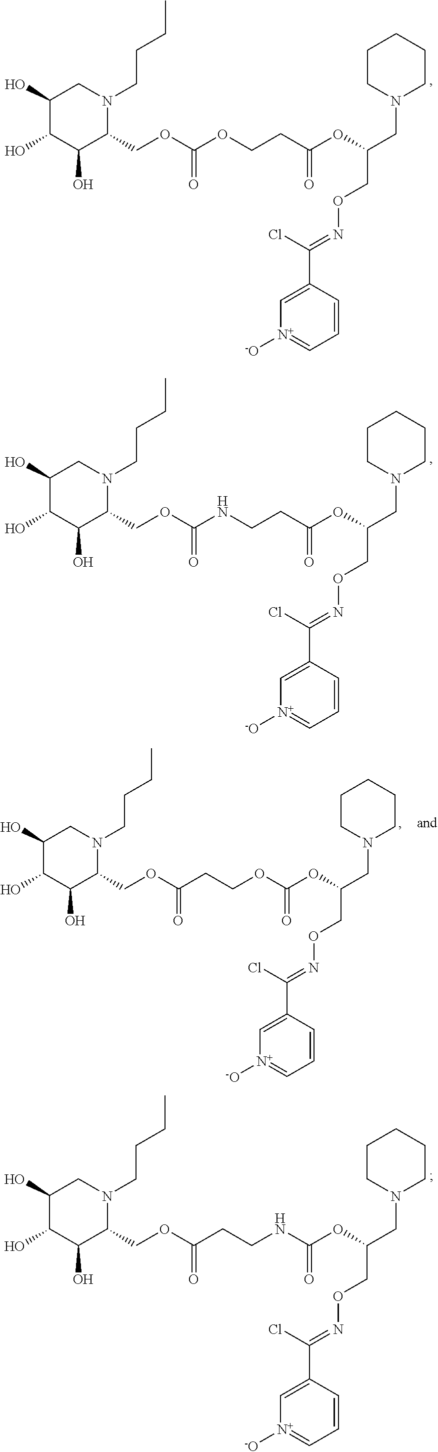

or a pharmaceutically acceptable salt thereof.

In a further aspect, the present technology relates to a method of treating a neurodegenerative or metabolic disorder in a patient in need thereof, the method comprising administering to the patient in need thereof a composition comprising a therapeutically effective amount of a compound of Formula I, wherein the compound of Formula I is present in a form selected from the group consisting of a stereoisomer and a racemate, or a mixture thereof.

In a further aspect, the present technology relates to a method of treating a neurodegenerative or metabolic disorder in a patient in need thereof, the method comprising administering to the patient in need thereof a composition comprising a therapeutically effective amount of a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further aspect, the present technology relates to a method of treating a neurodegenerative or metabolic disorder in a patient in need thereof, the method comprising administering to the patient in need thereof a composition comprising a therapeutically effective amount of a compound of Formula I, wherein X is N or O, and Y is N or O; or a pharmaceutically acceptable salt thereof. In a further aspect, the compound of Formula I is selected from the group consisting of:

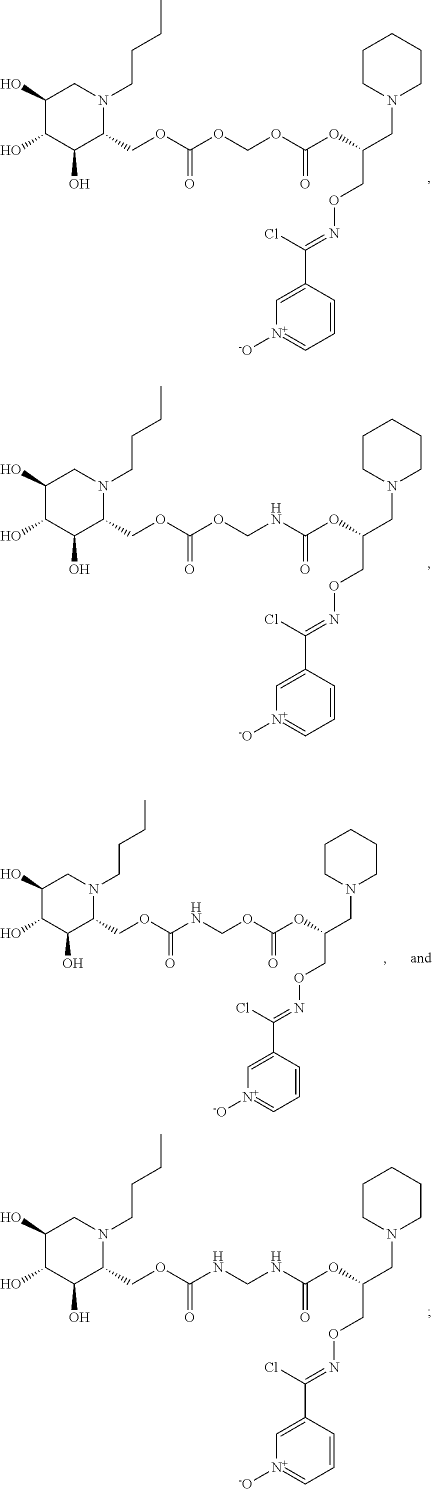

or a pharmaceutically acceptably salt thereof.

In a further aspect, the present technology relates to a method of treating a neurodegenerative or metabolic disorder in a patient in need thereof, the method comprising administering to the patient in need thereof a composition comprising a therapeutically effective amount of a compound of Formula I, wherein the compound of Formula I is present in a form selected from the group consisting of a stereoisomer and a racemate, or a mixture thereof.

In a further aspect, the present technology relates to a method of treating a neurodegenerative or metabolic disorder in a patient in need thereof, the method comprising administering to the patient in need thereof a composition comprising a therapeutically effective amount of a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a method of treating a neurodegenerative or metabolic disorder in a patient in need thereof, the method comprising:
administering to the patient in need thereof a composition comprising a therapeutically effective amount of a compound of Formula II:

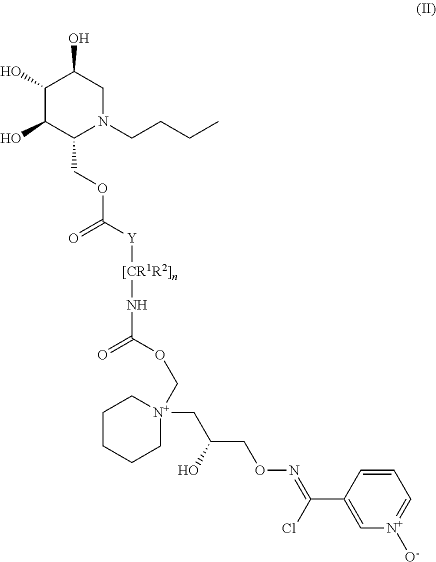

wherein Y is selected from the group consisting of O, N, or absent,
   n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a method of treating a neurodegenerative or metabolic disease or disorder in a patient in need thereof, the method comprising administering to the patient in need thereof a composition comprising a therapeutically effective amount of the compound of Formula II, wherein n is 1-3, alternatively 4-7, alternatively 8-10, alternatively 1-5, alternatively 3-7, alternatively 6-10

In a further aspect, the present technology relates to a method of treating a neurodegenerative or metabolic disease or disorder in a patient in need thereof, the method comprising administering to the patient in need thereof a composition comprising a therapeutically effective amount of the compound of Formula II, wherein Y is absent; or a pharmaceutically acceptable salt thereof. In a further aspect the compound of Formula II is selected from the group consisting of:

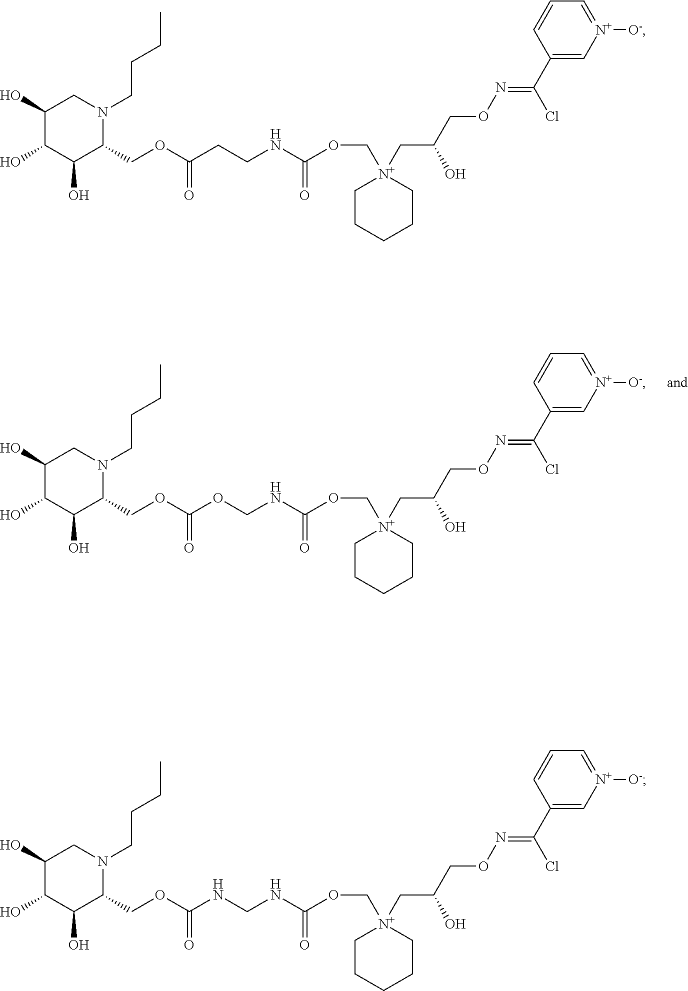

or a pharmaceutically acceptable salt thereof.

In a further aspect, the present technology relates to a method of treating a neurodegenerative or metabolic disease or disorder in a patient in need thereof, the method comprising administering to the patient in need thereof a composition comprising a therapeutically effective amount of the compound of Formula II, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a method of treating a neurodegenerative or metabolic disorder in a patient in need thereof, the method comprising:
administering to the patient in need thereof a therapeutically effective amount of a compound of Formula III:

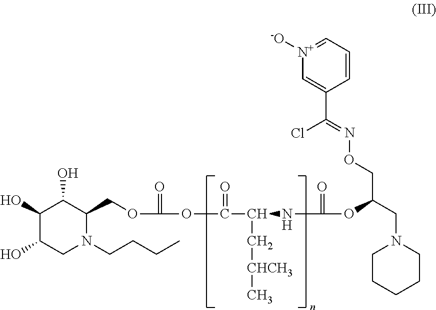

wherein n is 1-50,
or a pharmaceutically acceptable salt thereof.

In a further aspect, the present technology relates to a method of treating a neurodegenerative or metabolic disease or disorder in a patient in need thereof, the method comprising administering to the patient in need thereof a composition comprising a therapeutically effective amount of the compound of Formula III, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a method of treating a neurodegenerative or metabolic disease or disorder in a patient in need thereof, the method comprising administering to the patient in need thereof a composition comprising a therapeutically effective amount of the compound of Formula III, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a method of treating a neurodegenerative or metabolic disorder in a patient in need thereof, the method comprising:
administering to the patient in need thereof a therapeutically effective amount of a compound of Formula IV:

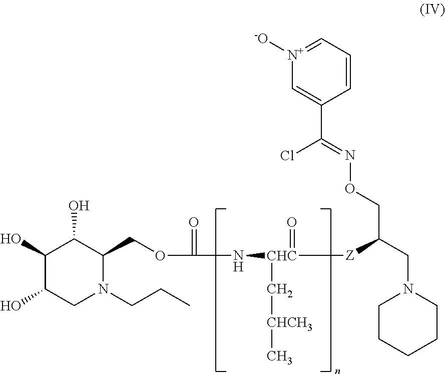

wherein Z is selected from the group consisting of O or OC(═O)O,
   n is 1-50,
or a pharmaceutically acceptable salt thereof.

In a further aspect, the present technology relates to a method of treating a neurodegenerative or metabolic disease or disorder in a patient in need thereof, the method comprising administering to the patient in need thereof ae composition comprising a therapeutically effective amount of the compound of Formula IV, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a method of treating a neurodegenerative or metabolic disease or disorder in a patient in need thereof, the method comprising administering to the patient in need thereof ae composition comprising a therapeutically effective amount of the compound of Formula IV, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a method of treating a neurodegenerative or metabolic disorder in a patient in need thereof, the method comprising:
administering to the patient in need thereof a therapeutically effective amount of a compound of Formula V:

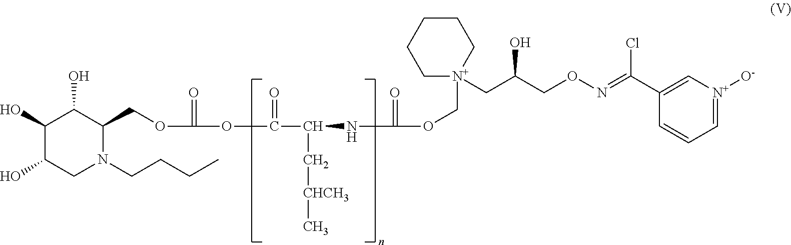

wherein n is 1-50,
or a pharmaceutically acceptable salt thereof.

In a further aspect, the present technology relates to a method of treating a neurodegenerative or metabolic disease or disorder in a patient in need thereof, the method comprising administering to the patient in need thereof a composition comprising a therapeutically effective amount of a compound of Formula V, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a method of treating a neurodegenerative or metabolic disease or disorder in a patient in need thereof, the method comprising administering to the patient in need thereof a composition comprising a therapeutically effective amount of a compound of Formula V, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the therapeutically effective amount of the compound selected from the group comprising Formula I, Formula II, Formula III, Formula IV, and Formula V, or mixtures thereof, ranges from about 1 to about 10000 mg per day.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the composition is formulated for parenteral or intranasal administration.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the composition is formulated for oral or suppository administration.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the composition is formulated for oral administration.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the oral formulation is a liquid oral dosage formulation.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the liquid oral formulation is selected from the group consisting of syrups, emulsions, solutions, and suspensions.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the oral formulation is a solid oral dosage formulation.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the solid oral dosage formulation is selected from the group consisting of a sublingual, a gummy, a chewable tablet, a rapidly dissolving tablet, a tablet, a capsule, a caplet, a troche, a lozenge, an oral powder, a solution, a thin strip, an oral thin film (OTF), and an oral strip.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the solid oral dosage is administered as a unit dosage.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the unit dosage is administered in 1 to 3 unit doses.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the neurodegenerative or metabolic disease or disorder is a lysosomal storage disorder.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the lysosomal storage disorder is selected from the group consisting of sphingolipidoses, mucopolysaccharidoses (MPS), lysosomal glycogen storage disorders, oligosaccharidoses, mucolipidoses, lipid storage disorders, glycoproteinoses, neuronal ceroid lipofuscinoses (NCL), peroxisomal biogenesis disorders (Zellweger spectrum disorders), and other lysosomal storage disorders.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the sphingolipidosis is selected from the group consisting of Gaucher disease type 1, Gaucher disease type 2, Gaucher disease type 3, Fabry disease, Niemann-pick disease type A, Niemann-pick disease type B, Niemann-pick disease type C, Tay-Sachs disease, Sandhoff disease, GM1 gangliosidosis, metachromatic leukodystrophy (MLD), Krabbe disease, and Farber lipogranulomatosis.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the mucopolysaccharidosis is selected from the group consisting of MPS I, Hurler syndrome, Hurler-Scheie syndrome, Scheie syndrome, MPS II, Hunter syndrome), MPS III, Sanfilippo syndrome type A, Sanfilippo syndrome type B, Sanfilippo syndrome type C, Sanfilippo syndrome type D, MPS IV, Morquio syndrome type A, Morquio syndrome type B), MPS VI, Maroteaux-Lamy syndrome, MPS VII, Sly syndrome, MPS IX, and Natowicz syndrome.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the lysosomal glycogen storage disorder is selected from the group consisting of Pompe disease and glycogen storage disease type II.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the oligosaccharidosis is selected from the group consisting of Schindler disease, Sialidosis Type I, Sialidosis type II, aspartylglucosaminuria, alpha-mannosidosis, beta-mannosidosis, and fucosidosis.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the mucolipidosis is selected from the group consisting of mucolipidosis II, I-cell disease, mucolipidosis III, pseudo-Hurler polydystrophy, and mucolipidosis IV.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the lipid storage disorder is selected from the group consisting of Wolman disease, cholesteryl ester storage disease, and Farber disease.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the glycoproteinosis is selected from the group consisting of alpha-mannosidosis, beta-mannosidosis, fucosidosis, aspartylglucosaminuria.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the neuronal ceroid lipofuscinosis is selected from the group consisting of infantile NCL, CLN1, CLN5, late infantile NCL, CLN2, juvenile NCL, CLN3, adult NCL, Kufs disease, CLN4, congenital NCL, and CLN10.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the peroxisomal biogenesis disorder is selected from the group consisting of Zellweger syndrome, neonatal adrenoleukodystrophy, and infantile Refsum disease.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the other lysosomal storage disorder is selected from the group consisting of Danon disease, cystinosis, sialic acid storage disease, Salla disease, lysosomal acid lipase deficiency (LAL-D), multiple sulfatase deficiency, pycnodysostosis, galactosialidosis, hyaluronidase deficiency (IPS IX), Hermansky-Pudlak syndrome, and pseudoxanthoma elasticum (PXE).

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the Niemann Pick disease Type C is Niemann Pick disease Type C 1 or Niemann Pick disease Type C 2.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the composition further comprises a therapeutically effective amount of at least one additional active pharmaceutical ingredient, wherein the at least one additional active pharmaceutical ingredient is selected from the group consisting of bimoclomol, miglustat, eliglustat, N-acetyl-L-leucine and 2-hydroxypropyl-β-cyclodextrin, arimoclomol, and combinations thereof.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the at least one additional active pharmaceutical ingredient is selected from miglustat, arimoclomol, N-acetyl-L-leucine, and combinations thereof.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the at least one additional active pharmaceutical ingredient is a combination of miglustat, arimoclomol, and N-acetyl-L-leucine.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the at least one additional active pharmaceutical ingredient is a combination of arimoclomol and miglustat.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the at least one additional active pharmaceutical ingredient is a combination arimoclomol and N-acetyl-L-leucine.

In a further aspect, the present technology relates to a method of treating a neurodegenerative or metabolic disorder in a patient in need thereof, the method comprising administering to the patient in need thereof a composition comprising a therapeutically effective amount of a compound selected from the group consisting of Formula I to Formula V, wherein the at least one additional pharmaceutical ingredient is a combination of miglustat and N-acetyl-L-leucine.

### DETAILED DESCRIPTION

In addition, any of the compositions or methods provided, disclosed, or described herein can be combined with one or more of any of the other compositions and methods provided, disclosed, or described herein.

Currently neurodegenerative or metabolic disorders such as Niemann-Pick disease type C and Gaucher Disease are treated by administering, alone or in combination, arimoclomol and miglustat. The standard dosing regimen comprises administering up to 3 unit doses per day in order to maintain effective plasma levels of the drugs.

Furthermore, brain absorption of miglustat is low, likely around 20%, which results in requiring a higher dosage to achieve therapeutic effect; and that the higher dosage requirement of miglustat results in gastrointestinal effects believed to be caused by inhibition of intestinal disaccharidases, which causes for example, carbohydrate malabsorption, diarrhea, abdominal pain, flatulence, bloating, vomiting, nausea, anorexia, dyspepsia, nutrient deficiency, weight loss, and stunted growth.

The inventors discovered that arimoclomol and miglustat can be joined together through at the 2-hydroxy position of arimoclomol and the 2-hydroxymethyl position of miglustat by substituents that are cleaved in vivo releasing both arimoclomol and miglustat in their unconjugated forms.

The presently described compounds unexpectedly display much better pharmacokinetics characterized by either lower maximum plasma concentrations (Cₘₐₓ) or Cₘₐₓ values comparable to unmodified arimoclomol and/or miglustat, longer times to maximum plasma concentration (Tₘₐₓ), and longer half-life (t_{1/2}). This results in a longer elimination phase with higher plasma concentrations over a longer period of time as measured by Area Under the Curve (AUC). These improved pharmacokinetics allow for dosages that result in therapeutically effective plasma levels for longer periods of time per dose, meaning patients need fewer doses per day, even only a single dose per day.

Reference will now be made in detail to exemplary embodiments of the claimed invention. While the claimed invention will be described in conjunction with the exemplary embodiments, it will be understood that it is not intended to limit the claimed invention to those embodiments. To the contrary, it is intended to cover alternatives, modifications, and equivalents, as may be included within the spirit and scope of the claimed invention, as defined by the appended claims.

Those of ordinary skill in the art may make modifications and variations to the embodiments described herein without departing from the spirit or scope of the claimed invention. In addition, although certain methods and materials are described herein, other methods and materials that are similar or equivalent to those described herein can also be used to practice the claimed invention.

### DEFINITIONS

“A” and “an” as it relates to the present technology, means the singular form, but includes the plural form unless clear from the context.

“About” as it relates to the present technology means, as it applied to measured quantities, +/−10% of the stated measured value; for example “about 100 mg” means 100 mg+/−10%, i.e. 90-110 mg. Unless specifically stated or obvious from context, as used herein, the term “about” is understood as within a range of normal tolerance in the art, for example, within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “or” means, and is used interchangeably with, the term “and/or,” unless context clearly indicates otherwise.

As used herein, the term “such as” means, and is used interchangeably with, the phrase “such as, for example” or “such as but not limited.”

As used herein, the term “subject” means a human or animal, including but not limited to a human or animal patient.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

“Arimoclomol” as it relates to the present technology means N-[2-hydroxy-3-(1-piperidinyl)-propoxy]-pyridine-1-oxide-3-carboximidoyl chloride, its stereoisomers and the acid addition salts thereof. Stereoisomers of arimoclomol include, but are not limited to (+)-(R)—N-[2-hydroxy-3-(1-piperidinyl)-propoxy]-pyridine-1-oxide-3-carboximidoyl chloride, (−)-(S)—N-[2-hydroxy-3-(1-piperidinyl)-propoxy]-pyridine-1-oxide-3-carboximidoyl chloride, (Z)—(R)—N-[2-hydroxy-3-(1-piperidinyl)-propoxy]-pyridine-1-oxide-3-carboximidoyl chloride, (E)-(R)—N-[2-hydroxy-3-(1-piperidinyl)-propoxy]-pyridine-1-oxide-3 carboximidoyl chloride, (Z)—(S)—N-[2-hydroxy-3-(1-piperidinyl)-propoxy]-pyridine-1-oxide-3-carboximidoyl chloride; and (E)-(S)—N-[2-hydroxy-3-(1-piperidinyl)-propoxy]-pyridine-1-oxide-3-carboximidoyl chloride.

“Cholesterol,” as it relates to the present technology means free cholesterol or cholest-5-en-30-ol. “Cholesterol” as it relates to the present technology includes and may be used interchangeably with “unesterified cholesterol” as defined below.

“CLEAR” means “Coordinated Lysosomal Expression and Regulation”.

“Drug” or “medicament” as it relates to the present technology includes biologically, physiologically, or pharmacologically active substances that act locally or systemically in the human or animal body.

“Esterified cholesterol” as it relates to the present technology means a form of cholesterol where the hydroxyl group of the cholesterol molecule has been conjugated with a fatty acid to form an ester-bond that links the cholesterol molecule and the fatty acid. In most animal tissues, an enzyme acyl-CoA:cholesterol acyltransferase (ACAT) or sterol O-acyl-transferase (SOAT) synthesizes cholesterol esters from CoA esters of fatty acids and cholesterol. ACAT exists in two forms, both of which are intracellular enzymes found in the endoplasmic reticulum and are characterized by multiple transmembrane domains and a catalytic histidine residue in a hydrophobic domain.

“Niemann-Pick disease type C Clinical Severity Scale” and/or “NPCCSS” as it relates to the present technology means a composite clinical severity scale (hereafter “NPCCSS”; see Yanjanin et al.). A full “17-domain NPCCSS score” incorporates clinical signs and symptoms in nine major (ambulation, cognition, eye movement, fine motor, hearing, memory, seizures, speech, swallowing) and eight minor (auditory brainstem response, behavior, gelastic cataplexy, hyperreflexia, incontinence, narcolepsy, psychiatric, respiratory problems) domains to determine a score which describes the severity of the subject's NPC progression (a higher score, the more progressed/severe the disease is). An abridged “5-domain NPCCSS score” is successfully used by clinicians and incorporates clinical signs and symptoms from the major domains of ambulation, cognition, fine motor, speech and swallowing (see Cortina-Borja). A “4-domain NPCCSS score” is used based on Ambulation, Fine Motor Skills, Speech, and Swallow domains.

“Impaired cholesterol trafficking” as it relates to the present technology means a reduced ability to remove cholesterol from the cell through the normal lysosomal pathway.

“Including,” as it relates to the present technology means, and is used interchangeably with, the phrase “including but not limited to.”

“Isoform 1” as it relates to the present technology means a mature form of NPC1 protein

“Lipid storage disorders” or “lipidoses” are a subgroup of the lysosomal storage disorders in which harmful amounts of lipids accumulate in the intracellular space due to reduced expression or function of the enzymes needed to metabolize lipids. Over time, this excessive storage of lipids can cause permanent cellular and tissue damage, particularly in the brain, peripheral nervous system, liver, spleen and bone marrow.

Lipids are a broad group of naturally occurring molecules which include fats, waxes, sterols, fat-soluble vitamins (such as vitamins A, D, E and K), monoglycerides, diglycerides, phospholipids, and others. The main biological functions of lipids include energy storage, as structural components of cell membranes, and as important signaling molecules.

Lipids may be broadly defined as hydrophobic or amphiphilic small molecules; the amphiphilic nature of some lipids allows them to form structures such as vesicles, liposomes, or membranes in an aqueous environment. Biological lipids originate entirely or in part from two distinct types of biochemical subunits: ketoacyl and isoprene groups. Using this approach, lipids may be divided into eight categories: fatty acyls, glycerolipids, glycerophospholipids, sphingolipids, saccharolipids and polyketides (derived from condensation of ketoacyl subunits); and sterol lipids and prenol lipids (derived from condensation of isoprene subunits).

Although the term lipid is sometimes used as a synonym for fats, fats are a subgroup of lipids called triglycerides. Lipids also encompass molecules such as fatty acids and their derivatives (including tri-, di-, and monoglycerides and phospholipids), as well as other sterol-containing metabolites such as cholesterol.

Several lysosomal storage disorders characterized by the accumulation of lipids (i.e., lipid storage disorders) have been characterized; these are outlined herein below.

“Lysosomal Sphingolipid Hydrolysis” as it relates to the present technology means a multitude of enzymes are involved in the lysosomal catabolism of sphingolipids (or glycophingolipids). These enzymes, or more specifically hydrolases, are each responsible for the degradation of a specific sphingolipid.

The lysosomal sphingolipid hydrolases interact with sphingolipid activator proteins (SAP or saposins) to stimulate the activity of said hydrolases. SAPs are considered to facilitate the enzyme/substrate interaction between water-soluble enzymes and membrane-bound substrates.

Further, the lipid composition of late endosomal and lysosomal compartments is characterized by the presence of negatively charged phospholipids such as BMP and PI (phosphatidylinositol), which also stimulates the activity of some hydrolases. The BMP-dependent lysosomal hydrolases include sialidase, α-galactosidase A, glucosylceramidase, β-galactosylceramidase, arylsulfatase A, acid ceramidase and Sphingomyelinase.

“Maturation,” “maturing,” or “mature” as it relates to the present technology means the process and/or characteristic of a protein reaching its folded state and optionally being subjected to post-translational modifications including, for example, glycosylation.

“Mechanism of Action of Arimoclomol” or “Arimoclomol MOA” as it relates to the present technology means the biological pathway by which arimoclomol treats the NPC Disease State. Arimoclomol targets NPC etiology by both NPC1-independent and NPC1-dependent pathways: (1) via the NPC1-independent pathway, arimoclomol upregulates expression of certain CLEAR genes other than NPC1, thereby improving overall cell health by, for example, increasing lysosomal biogenesis and/or autophagy flux; (2) via the NPC1-dependent pathway, CLEAR gene upregulation increases production of the NPC1 protein. Though still mutated, overproducing the reduced function protein improves lysosomal function and cholesterol elimination.

“Myelin Basic Protein” and/or “MBP” as it relates to the present technology means a protein believed to be important in the process of myelination of nerves in the nervous system. The myelin sheath is a multi-layered membrane, unique to the nervous system, that functions as an insulator to greatly increase the velocity of axonal impulse conduction. MBP maintains the correct structure of myelin, interacting with the lipids in the myelin membrane.

“Patient” as it relates to the present technology means a human or animal subject in need of treatment.

“Pharmaceutically acceptable salt” as it relates to the present technology means a salt which is not harmful to the subjects. Such salts include pharmaceutically acceptable basic or acid addition salts as well as pharmaceutically acceptable metal salts, ammonium salts and alkylated ammonium salts.

Acid addition salts include, but are not limited to, salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 66, 2, (1977) which is incorporated herein by reference.

Other pharmaceutically acceptable salts include, but are not limited to, acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, or undecylenate. In the preferred embodiments, the anionic salt form is selected from the group consisting of chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite.

“Rearing” as it relates to the present technology means a functional behavior in rodents that plays an important role in exploring and interacting with the environment. As such, rearing is a complex behavior that involves aspects such as locomotion, balance, exploratory drive, spatial awareness, cognitive mapping, sequence learning, and decision making. Therefore, the ability to rear is not simply an indicator of muscle function but a broader marker of brain health in rodents. The same brain regions (particularly the cerebellum, hippocampus, and midbrain) that are responsible for controlling rearing behavior in rodents are involved in recruiting muscles during movements related to fine motor function and swallow in humans. Rearing activity in NPC mice is therefore an appropriate indicator of neuronal health in brain regions relevant to functional endpoints like the Fine Motor Skills and Swallow domains of the NPCCSS in human NPC patients

“Saposin Deficiency” as it relates to the present technology means a disease (in both humans and mice) caused by prosaposin/saposin deficiencies which leads to severe neurological deficits. Human patients with point mutations in the saposin A, B and C show phenotypes of Krabbe disease, metachromatic leukodystrophy and Gaucher disease, indicating that their primary in vivo substrates are galactosylceramide, sulfatide and glucosylceramide, respectively. Krabbe disease, atypical, due to saposin A deficiency: An inherited biochemical disorder which results in neurological regression within a few months of birth. Death usually occurs during the first few years of life. The disorder is similar to Krabbe disease but is differentiated by the genetic origin of the biochemical defect. Krabbe disease involves a defect in the galactocerebrosidase gene whereas atypical Krabbe disease involves a defect in the prosaposin gene which causes a deficiency of saposin A. Saposin B, previously known as SAP-1 and sulfatide activator, stimulates the hydrolysis of a wide variety of substrates including cerebroside sulfate, GM1 ganglioside, and globotriaosylceramide by arylsulfatase A, acid beta-galactosidase, and alpha-galactosidase, respectively. Human saposin B deficiency, transmitted as an autosomal recessive trait, results in tissue accumulation of cerebroside sulfate and a clinical picture resembling metachromatic leukodystrophy (activator-deficient metachromatic leukodystrophy) although with normal arylsulfatase activity. Saposin B deficiency is a heterogeneous disease with a spectrum similar to that in metachromatic leukodystrophy. Saposin (SAP-) C is required for glucosylceramide degradation, and its deficiency results in a variant form of Gaucher disease; non-neuronopathic Gaucher disease due to SAP-C deficiency. Very high levels of chitotriosidase activity, chemokine CCL18, and increased concentration of glucosylceramide in plasma and normal β-glucosidase activity in skin fibroblasts are observed in the patients. A missense mutation, p.L349P, located in the SAP-C domain and another mutation, p.M1L, located in the initiation codon of the prosaposin precursor protein has been identified. In a few non-neuronopathic Gaucher patients, a mutation in both Saposin C and saposin D has been identified. Combined saposin C and D deficiencies in mice lead to a neuronopathic phenotype with glucosylceramide and alpha-hydroxy ceramide accumulation. In mice, saposin D deficiency is associated with ceramide accumulation, partial loss of Purkinje cells and impaired urinary system function. This phenotype does not mimic the embryonic lethality exhibited by mice with complete deficiency of acid ceramidase, saposin D's cognate enzyme. Two mutations are known in humans that result in complete inactivation of all four saposins and prosaposin. Total saposin deficiency is a devastating disease with involvement of multiple organs and multiple sphingolipids. Combined saposin deficiency (or prosaposin deficiency) has been reported in a case presenting with a severe neurovisceral dystrophy which caused death as a neonate. Multiple sphingolipids were elevated in the urine, with globotriaosylceramide showing the greatest increase. A novel mutation in the PSAP gene was identified, being homozygous for a splice-acceptor site mutation two bases upstream of exon 10. This mutation led to a premature stop codon and yielded low levels of transcript.

“Transcription Factor EB” as it relates to the present technology means protein that in humans is encoded by the TFEB gene. TFEB is a master gene for lysosomal biogenesis. It encodes a transcription factor that coordinates expression of lysosomal hydrolases, membrane proteins and genes involved in autophagy. Upon nutrient depletion and under aberrant lysosomal storage conditions such as in lysosomal storage diseases, TFEB translocate from the cytoplasm to the nucleus, resulting in the activation of its target genes.

“Transcription Factor E3” as it relates to the present technology means a protein that in humans is encoded by the TFE3 gene. TFE3, like TFEB, binds to CLEAR (Coordinated Lysosomal Expression and Regulation) motifs in the promoter regions of lysosomal and autophagy-related genes, thereby activating genes involved in lysosomal biogenesis and autophagy.

“Treating” or ‘treatment” as it relates to the present technology means any of the following: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Unesterified cholesterol” as it relates to the present technology means “free cholesterol,” or cholest-5-en-30-ol.

“Upregulate,” “upregulation,” or “upregulates” as it relates to the present technology means a process by which gene expression increases. Upregulation can be determined by measuring an increase in the presence of nucleotide strands such as DNA and/or RNA or an increase of the quantity of protein encoded by the gene which is expressed by the cell.

### Hydroxylamine-Hydroxypiperidiene Compounds

In an embodiment, the present disclosure provides a compound of Formula I:

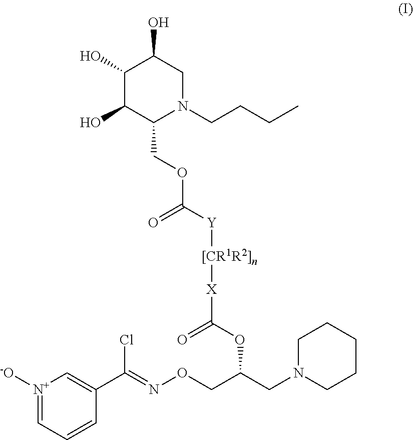

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkyl carb onyl amino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, aryl carb onyl amino, arylcarbonyloxy, arylcycloalkyl, aryloxy, aryloxyalkyl, arylsulfinyl, arylsulfinylalkyl, arylsulfonyl, aryl sulfonyl amino, arylthio, arylthioalkyl, cycloalkenyl, cycloalkenylalkyl, cycloalkyl, cycloalkylalkyl, cycloalkyl amino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroaryl amino, heteroarylcarbonyl, heteroaryl carb onyl amino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure describes a compound of Formula I, wherein n is 1-3, alternatively 4-7, alternatively 8-10, alternatively 1-5, alternatively 3-7, alternatively 6-10.

In a further embodiment, the present disclosure provides a compound of Formula I, wherein X and Y are absent; or a pharmaceutically acceptable salt thereof. Alternatively, the compound of Formula I is selected from the group consisting of:

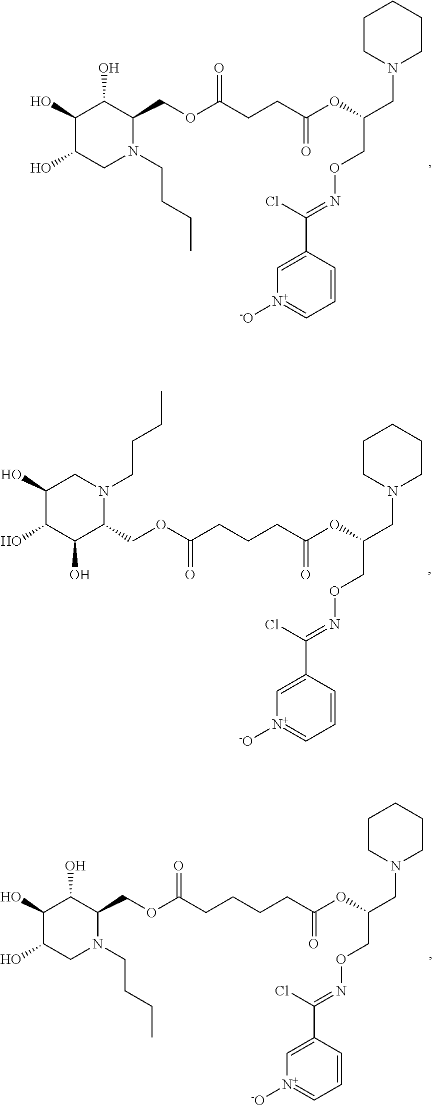

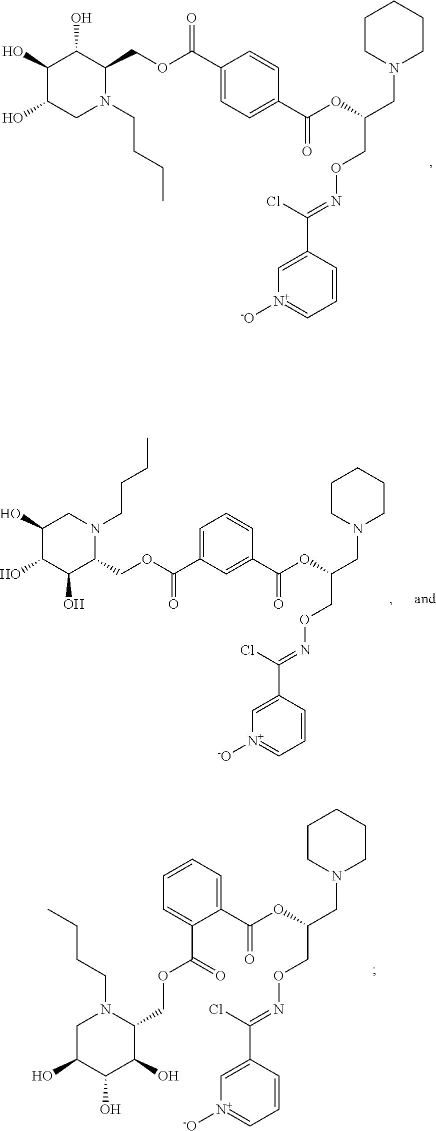

or a pharmaceutically acceptable salt thereof.

In a further embodiment, the present disclosure provides a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further embodiment, the present disclosure provides a compound of Formula I, wherein one of X or Y is absent; or a pharmaceutically acceptable salt thereof. Alternatively, the compound of Formula I is selected from the group consisting of:

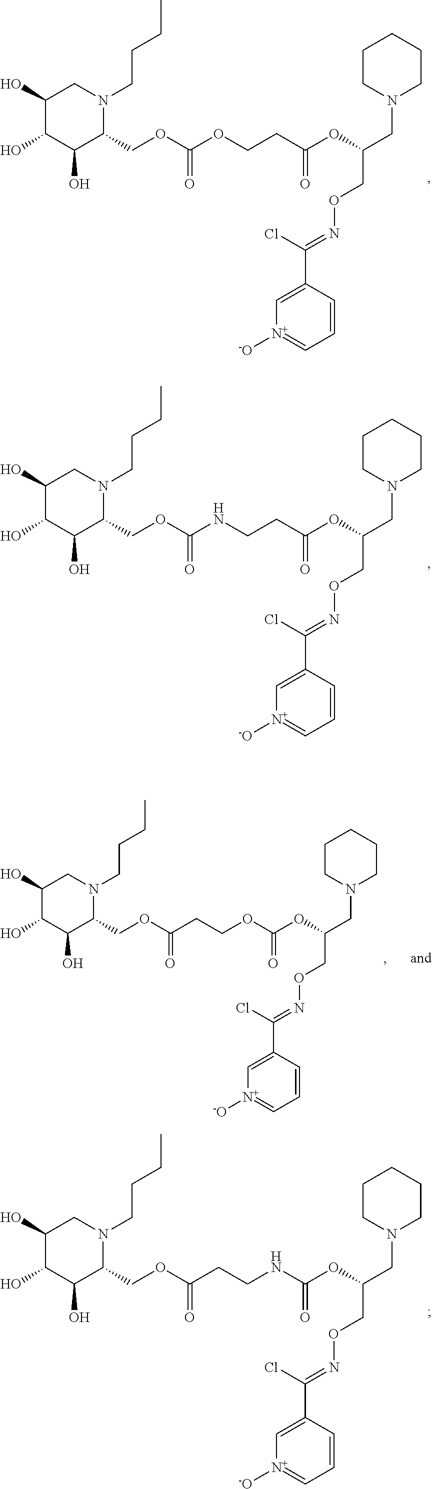

or a pharmaceutically acceptable salt thereof.

In a further embodiment, the present disclosure provides a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further embodiment, the present disclosure provides a compound of Formula I, wherein X is N or O, and Y is N or O; or a pharmaceutically acceptable salt thereof. Alternatively, the compound of Formula I is selected from the group consisting of.

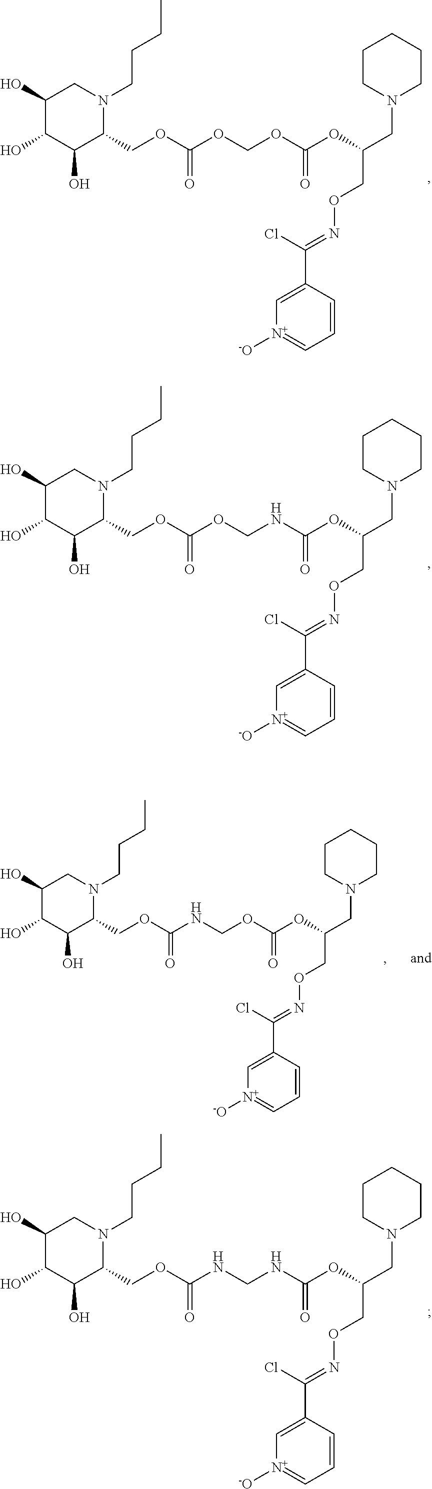

or a pharmaceutically acceptable salt thereof.

In a further embodiment, the present disclosure provides a compound of Formula I, wherein the compound of Formula I is present in a form selected from the group consisting of a stereoisomer and a racemate, or a mixture thereof.

In a further embodiment, the present disclosure provides a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In an embodiment, the present disclosure provides a compound of Formula II:

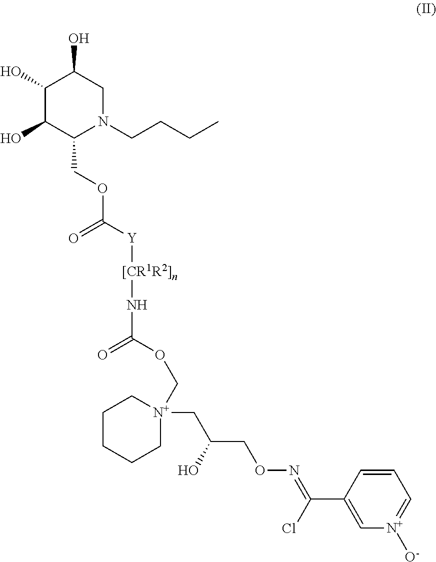

wherein Y is selected from the group consisting of O, N, or absent,
n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides a compound of Formula II, wherein n is 1-3, alternatively 4-7, alternatively 8-10, alternatively 1-5, alternatively 3-7, alternatively 6-10.

In a further embodiment, the present disclosure provides a compound of Formula II, wherein Y is absent; or a pharmaceutically acceptable salt thereof. Alternatively, the compound of Formula II is selected from the group consisting of:

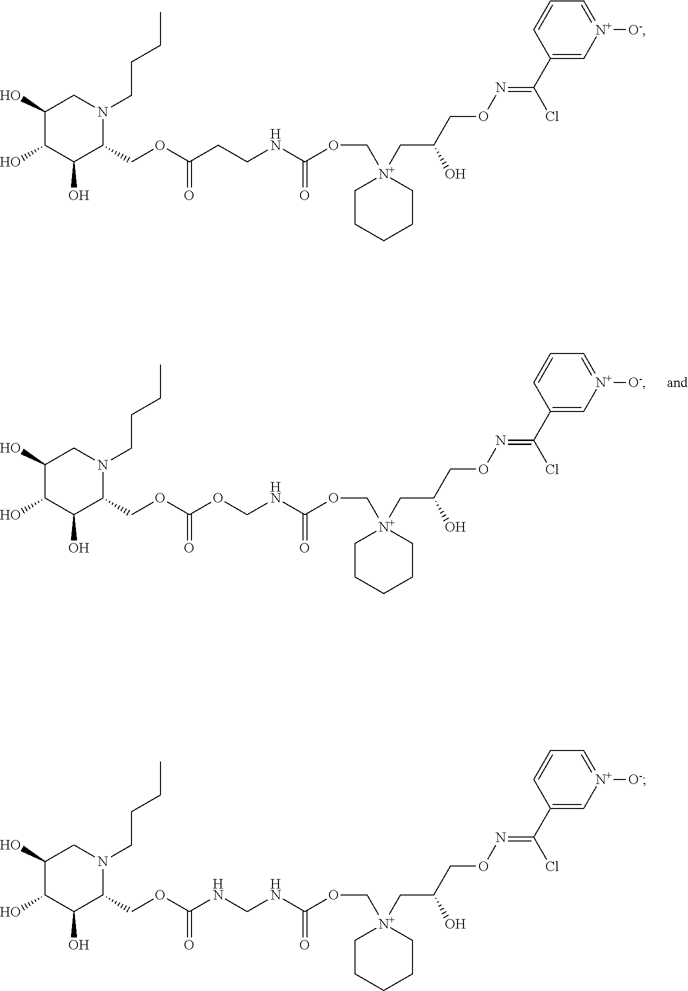

or a pharmaceutically acceptable salt thereof.

In a further embodiment, the present disclosure provides a compound of Formula II, wherein the compound of Formula II is present in a form selected from the group consisting of a stereoisomer and a racemate, or a mixture thereof.

In a further embodiment, the present disclosure provides a compound of Formula II, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In an embodiment, the present disclosure provides a compound of Formula III:

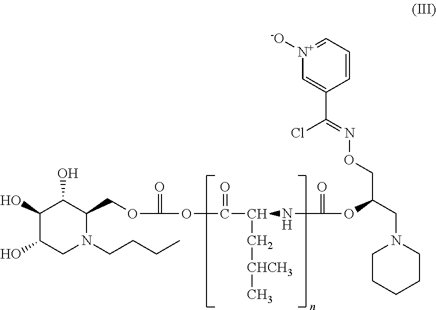

wherein n is 1-50,
or a pharmaceutically acceptable salt thereof.

In a further embodiment, the present disclosure provides a compound of Formula III, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present disclosure provides a compound of Formula III, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In an embodiment, the present disclosure provides to a compound of Formula IV:

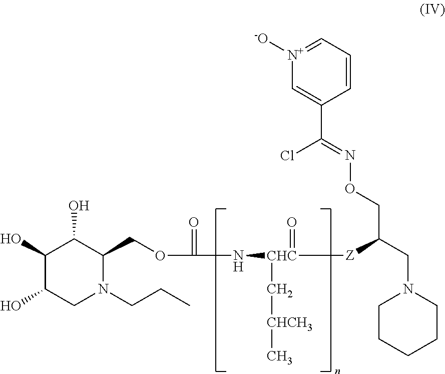

wherein Z is selected from the group consisting of O or OC(═O)O,
n is 1-50,
or a pharmaceutically acceptable salt thereof.

In a further embodiment, the present disclosure provides to a compound of Formula IV, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present disclosure provides to a compound of Formula IV, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In an embodiment, the disclosure provides a compound of Formula V:

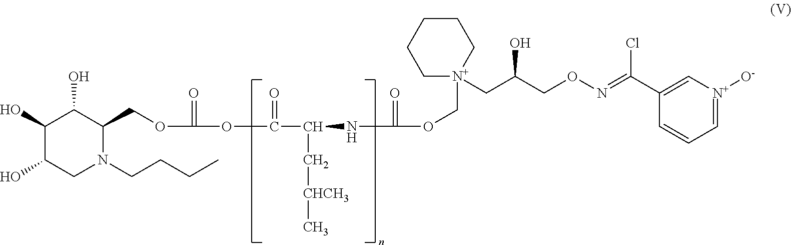

wherein n is 1-50,
or a pharmaceutically acceptable salt thereof.

In a still further aspect, the disclosure provides a compound of Formula V, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the disclosure provides a compound of Formula V, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

### Synthesis of Hydroxylamine-Hydroxypiperidine Conjugates

The compounds disclosed above are generally synthesized by joining arimoclomol and miglustat together via carbonyl groups linked by the —X—[CR¹R²]ₙ—Y— linker groups of Formula I and Formula II or the leucine polypeptide chains of Formula III, Formula IV, or Formula V according to the following schemes.

### Synthesis of Tribenzyl-Protected Miglustat (4)

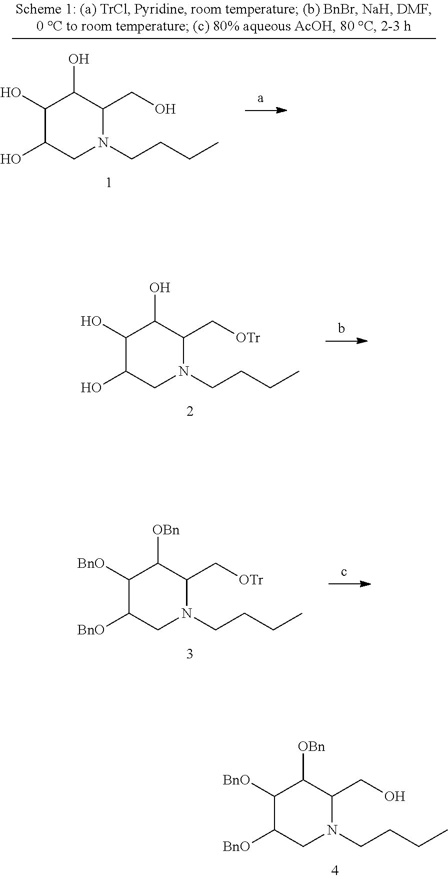

### Synthesis of Compound 2:

To a solution of miglustat 1 (2.5 g, 11.4 mmol, 1.0 eq) in anhydrous pyridine (20 mL) was added trityl chloride (TrCl, 3.5 g, 12.5 mmol) under argon atmosphere. The reaction mixture was stirred overnight at room temperature. Pyridine was removed under reduced pressure (in water bath at 28 °C). Any residual pyridine was removed by co-distilling the crude reaction mixture with toluene (2×30 mL). The resulting dark brown crude oil was diluted with dichloromethane (DCM) (100 mL) and the organic part was washed with 5% aq. NaHCO₃ solution (50 mL) and brine (50 mL), dried over Na₂SO₄, and evaporated to dryness to give the tritylated product. The crude product was purified over silica gel to afford trityl-miglustat 2 (1.74 g, 33%) as white solid.

### Synthesis of Compound 3:

Compound 2 (5.4 g, 11.7 mmol, 1.0 eq) was dissolved into anhydrous DMF (60 mL) and the solution was cooled to 0-5 °C with an ice-water bath under the argon atmosphere. Sodium hydride (90%, 1.56 g, 58.5 mmol,) was added in portions to the reaction mixture over 10 min. under constant stirring. After 30 min., a solution of benzyl bromide (6.3 mL, 52.6 mmol, 4.5 eq) in anhydrous DMF (30 mL) was added dropwise over a period of 30 min. (under the argon atmosphere) under constant stirring. Once the addition was completed, the ice-water bath was removed to allow the reaction mixture to gradually warm up to room temperature. After stirring overnight at room temperature, the reaction mixture was cooled to 0-5 °C with an ice-water bath and 100 mL of saturated ammonium chloride (NH₄Cl) was added. The reaction mixture was extracted with ethyl acetate (EtOAc) (2×100 mL). The combined organic phases were dried over anhydrous Na₂SO₄ and concentrated under reduced pressure. The crude product was purified over silica gel to give compound 3 (7.3 g, 85%) as a sticky solid.

### Synthesis of Compound 4:

A suspension of compound 3 (7.1 g, 9.7 mmol) in 80% acetic acid (200 mL) was heated at 80 °C for 3 h. The reaction mixture was cooled to room temperature and solvent was evaporated under reduced pressure. The residue was co-evaporated with toluene (25 mL) to remove any residual acetic acid. The resulting brown crude oil was diluted with ethyl acetate (200 mL) and the organic part was washed with 5% aq. NaHCO₃ solution (2×75 mL) and water (50 mL). The organic phase was dried over anhydrous Na₂SO₄ and evaporated to dryness to produce tribenzyl-miglustat conjugate 4. The crude product was purified by flash column chromatography (MeOH/DCM as the eluents) to afford the compound 4 (3.9 g, 82%) as a light brown oil which solidified over time.

### Synthesis of Compound 8:

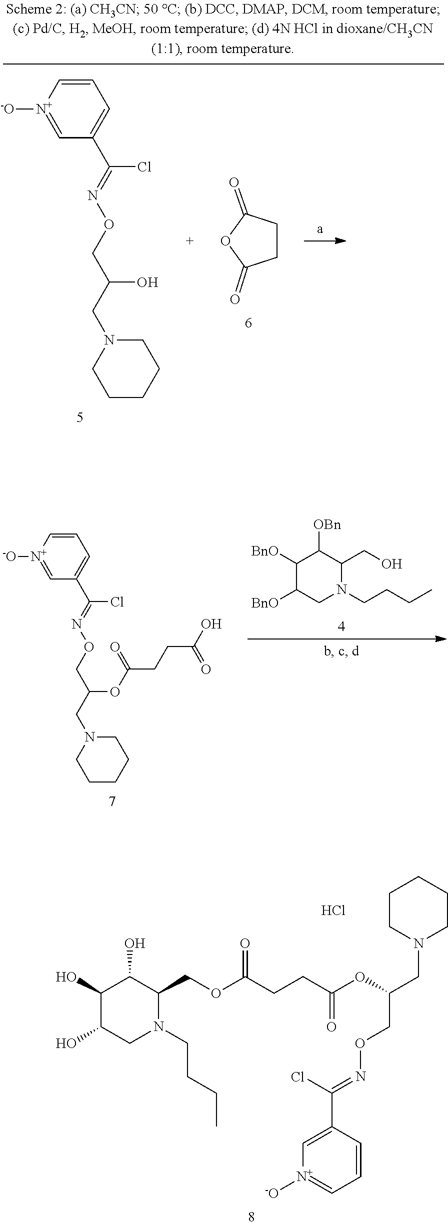

Synthesis of compound 7:

A solution of arimoclomol free base 5 (1 eq) and acid anhydride 6 (1.2 eq) in CH₃CN was heated at 50 °C for 1-2 h. The solvent evaporated under reduced pressure and the residue dried under vacuum to give 7.

### Synthesis of Compound 8:

To a solution of 7 (1 mmol), 4 (1.1-1.2 mmol), and DMAP (0.1 mmol) in DCM (10 mL) is added a solution of DCC (1.1-1.2 mmol) dropwise at room temperature. The reaction mixture is stirred at room temperature overnight and the suspension is filtered. The filtrate is evaporated to dryness and the crude product is purified by preparative HPLC to give the protected intermediate of 8.

A suspension of the protected intermediate of 8 (1 mmol), Pd/C (10% Pd, 100% wt/wt) in MeOH (20 mL/mmol) is stirred under hydrogen for 20 h at room temperature. The suspension is filtered through celite, washed with MeOH (2×10 mL) and the combined filtrates are evaporated under reduced pressure. The resulting residue is dried overnight under high vacuum.

The dry, deprotected product is dissolved in CH₃CN (5 mL) and to the solution is added 4N HCl/dioxane (5 mL). After stirring at room temperature for 1 h, solvent is evaporated under vacuum. The residue is co-evaporated with IPAc and dried to give the corresponding carbonate hydrochloride salt 8.

### Synthesis of Compound 10:

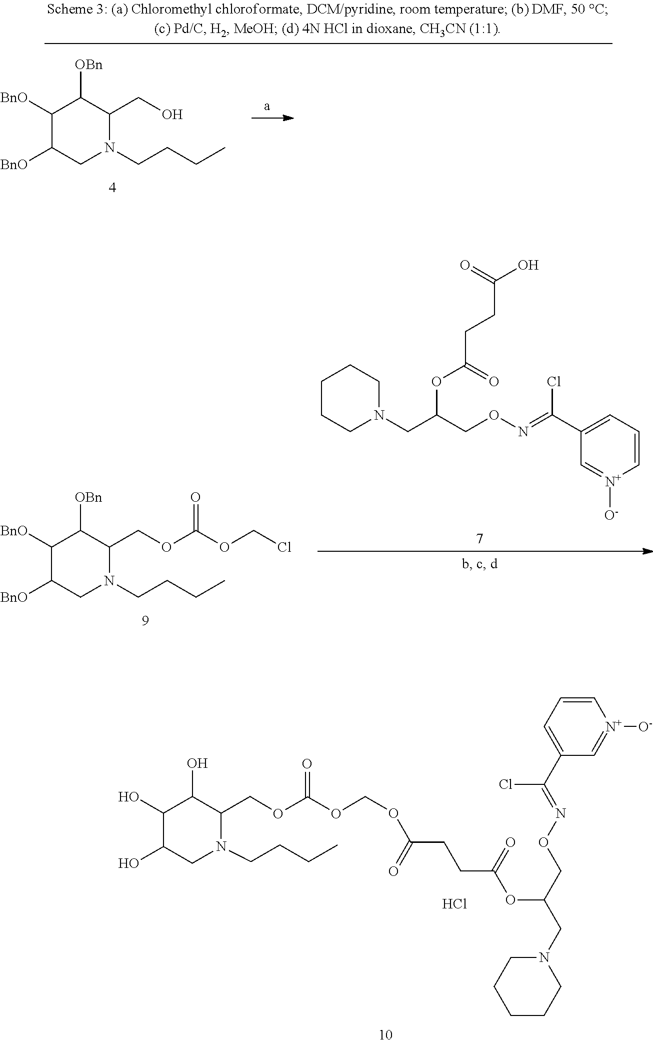 [SMILES: CCCCN1CC(OCc2ccccc2)C(OCc2ccccc2)C(OCc2ccccc2)C1CO ; CCCCN1CC(OCc2ccccc2)C(OCc2ccccc2)C(OCc2ccccc2)C1COC(=O)OCCl ; O=C(O)CCC(=O)OC(CO/N=C(\Cl)c1ccc[n+]([O-])c1)CN1CCCCC1 ; CCCCN1CC(O)C(O)C(O)C1COC(=O)OCOC(=O)CCC(=O)OC(CO/N=C(\Cl)c1ccc[n+]([O-])c1)CN1CCCCC1 ; Cl]

### Synthesis of Compound 9:

Compound 4 (1 mmol) was dissolved in DCM/pyridine (9:1, 10 mL) and to this solution was added a solution of chloromethyl chloroformate (2 mmol) in DCM (2 mL). After stirring for 2 at room temperature, solvent was evaporated under reduced pressure at 25 °C. The residual mixture was diluted with DCM (40 mL), and washed with 5% aq. NaHCO₃ and brine. The organic part was dried over Na₂SO₄, and solvent was evaporated under reduced pressure to give chloromethyl carbonate 9 which was used for next step without purification.

### Synthesis of Compound 10:

A suspension of compound 9 (1 mmol) and 7 (1.2 mmol) in DMF (5 mL) is heated for 2-6 h at 50 °C. DMF is removed under reduced pressure. The residue is dissolved in EtOAc (50 mL) and the EtOAc part is washed with 5% aq. NaHCO₃ (25 mL) and brine, dried over Na₂SO₄, and evaporated to dryness. The crude product is purified by preparative HPLC to give the protected intermediate of 10.

A suspension of the protected intermediate of 10 (1 mmol) and Pd/C (10% Pd, 100% wt/wt) in MeOH (20 mL) is stirred under hydrogen for 20 h. The suspension is filtered through celite and washed with MeOH (2×10 mL). The combined filtrates are evaporated under reduced pressure and the residue is dried over high vacuum.

The dry, deprotected product is dissolved in CH₃CN (5 mL) and to the solution was added 4N HCl/dioxane (5 mL). After stirring at room temperature for 1 h, solvent is evaporated under vacuum. The residue is co-evaporated with IPAc and dried to give the corresponding hydrochloride salt 10.

### Synthesis of Compound 14:

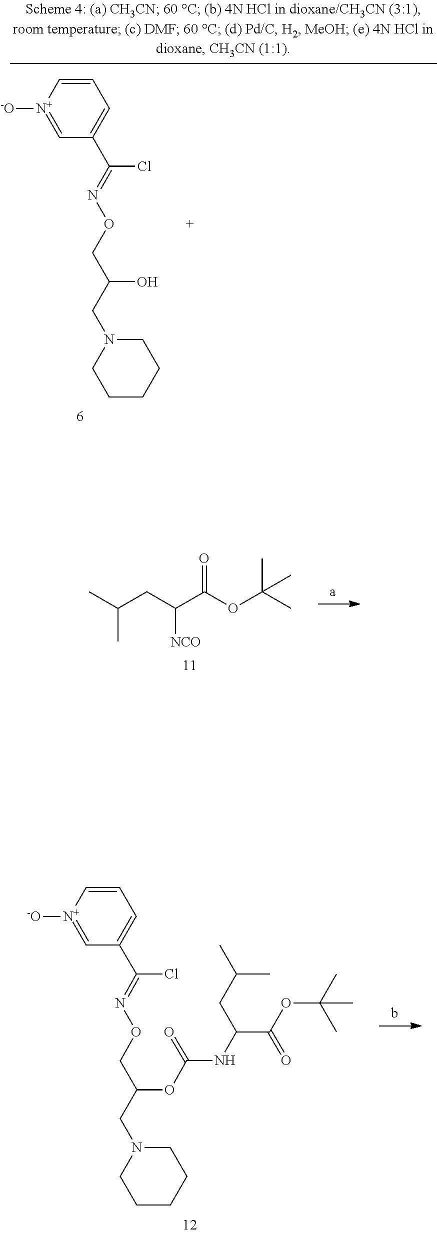

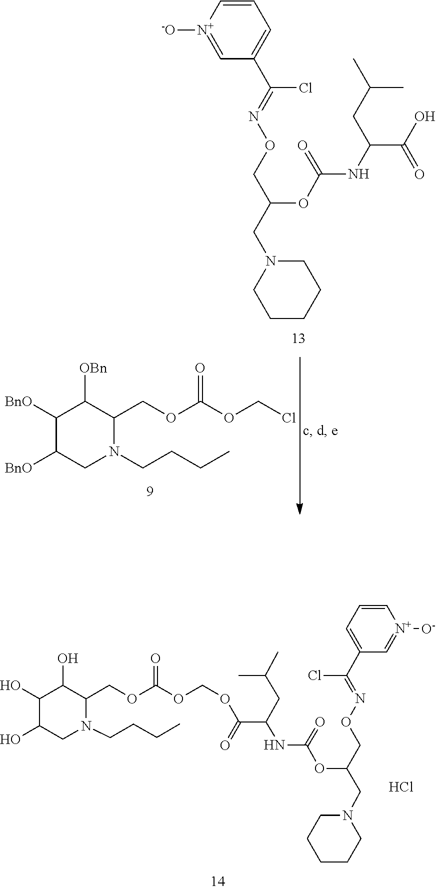

### Synthesis of Compound 12:

A solution of arimoclomol free base 6 (1 eq) and L-leucine tert-butyl ester isocyanate (1.5 eq) in CH₃CN (5 mL) is heated at 60 °C for 6-8 h. Solvent is evaporated, and the crude product is purified by preparative HPLC to afford 12.

### Synthesis of Compound 13:

To a solution of 12 in 1,4-dioxane (3 mL) is added 4N HCl/dioxane (9 mL) and the reaction mixture is stirred overnight at room temperature. The solvent is evaporated under vacuum. The resulting residue is co-evaporated with IPAc and dried to give 13.

### Synthesis of Compound 14:

A suspension of compound 13 (1 mmol) and 9 (1.2 mmol) in DMF (5 mL) is heated for 2-6 h at 60 °C. DMF is removed under reduced pressure. The residue is dissolved in EtOAc (50 mL) and the EtOAc part is washed with 5% aq. NaHCO₃ (25 mL) and brine, dried over Na₂SO₄, and evaporated to dryness. The crude product is purified by preparative HPLC to give the protected intermediate of 14.

A suspension of the protected intermediate of 14 (1 mmol) and Pd/C (10% Pd, 100% wt/wt) in MeOH (20 mL) is stirred under hydrogen for 20 h. The suspension is filtered through celite and washed with MeOH (2×10 mL). The combined filtrates are evaporated under reduced pressure and the residue is dried over high vacuum.

The dry, deprotected product is dissolved in CH₃CN (5 mL) and to the solution is added 4N HCl/dioxane (5 mL). After stirring at room temperature for 1 h, solvent is evaporated under vacuum. The residue is co-evaporated with IPAc and dried to give the corresponding hydrochloride salt 14.

### Pharmaceutical Compositions Comprising Hydroxylamine-Hydroxypiperdine Compounds

“Pharmaceutical composition” as it relates to the present technology means a composition comprising at least one active pharmaceutical ingredient (API) and, optionally, one or more excipients as defined herein.

In an embodiment, the present disclosure describes a composition comprising:
a therapeutically effective amount of a compound of Formula I:

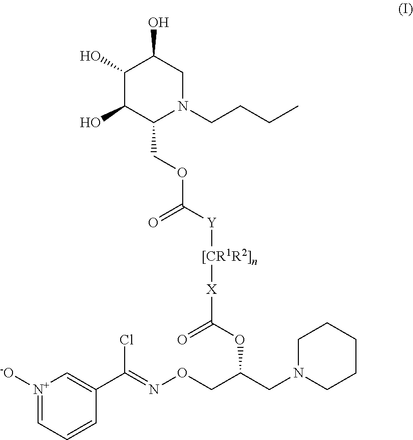

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides a composition comprising a therapeutically effective amount of a compound of Formula I, wherein n is 1-3, alternatively 4-7, alternatively 8-10, alternatively 1-5, alternatively 3-7, alternatively 6-10.

In a further embodiment, the present disclosure provides a composition comprising a therapeutically effective amount of a compound of Formula I, wherein X and Y are absent; or a pharmaceutically acceptable salt thereof. Alternatively, the compound of Formula I is selected from the group consisting of.

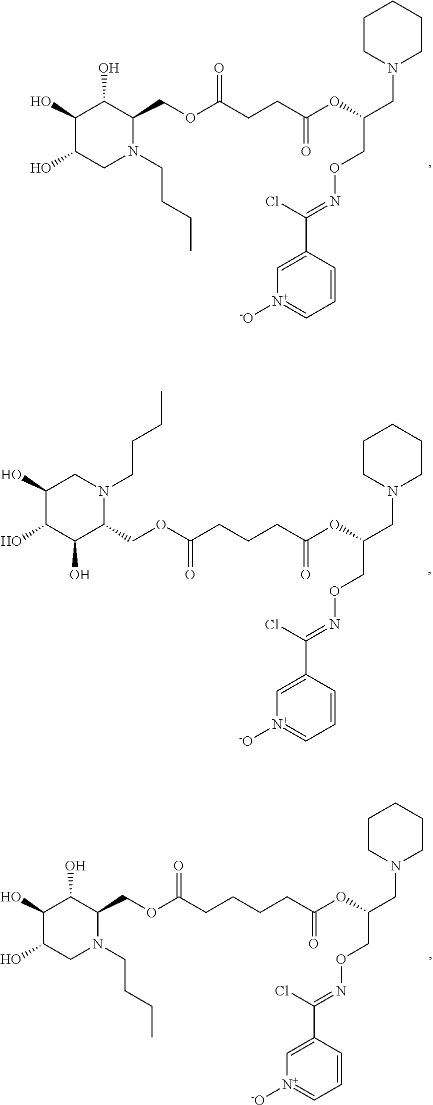

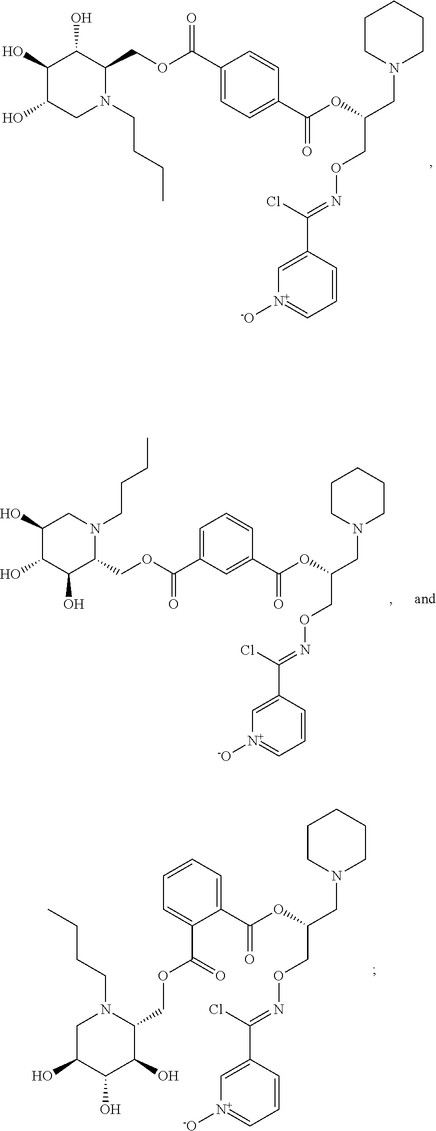

or a pharmaceutically acceptable salt thereof.

In a further embodiment, the present disclosure provides a composition comprising a therapeutically effective amount of a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further embodiment, the present disclosure provides a composition comprising a therapeutically effective amount of a compound of Formula I, wherein X or Y is absent; or a pharmaceutically acceptable salt thereof. Alternatively the compound of Formula I is selected from the group consisting of.

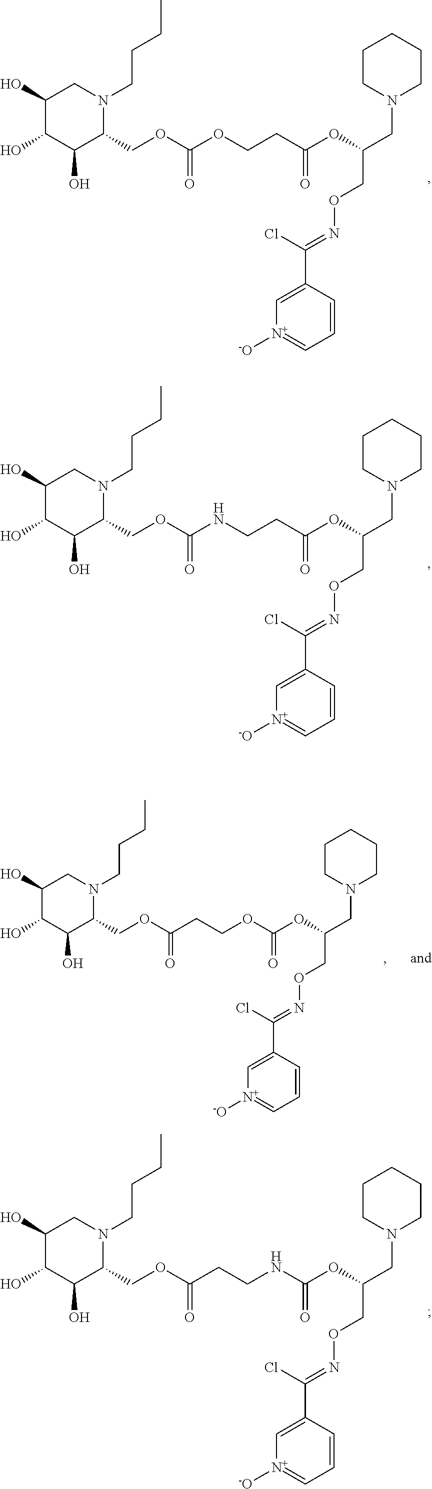

or pharmaceutically acceptable salt thereof.

In a further embodiment, the present disclosure provides a composition comprising a therapeutically effective amount of a compound of Formula I, wherein the compound of Formula I is present in a form selected from the group consisting of a stereoisomer and a racemate, or a mixture thereof.

In a further embodiment, the present disclosure provides a composition comprising a therapeutically effective amount of a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further embodiment, the present disclosure provides a composition comprising a therapeutically effective amount of a compound of Formula I, wherein X is N or O, and Y is N or O; or a pharmaceutically acceptable salt thereof. Alternatively, the compound of Formula I is selected from the group consisting of.

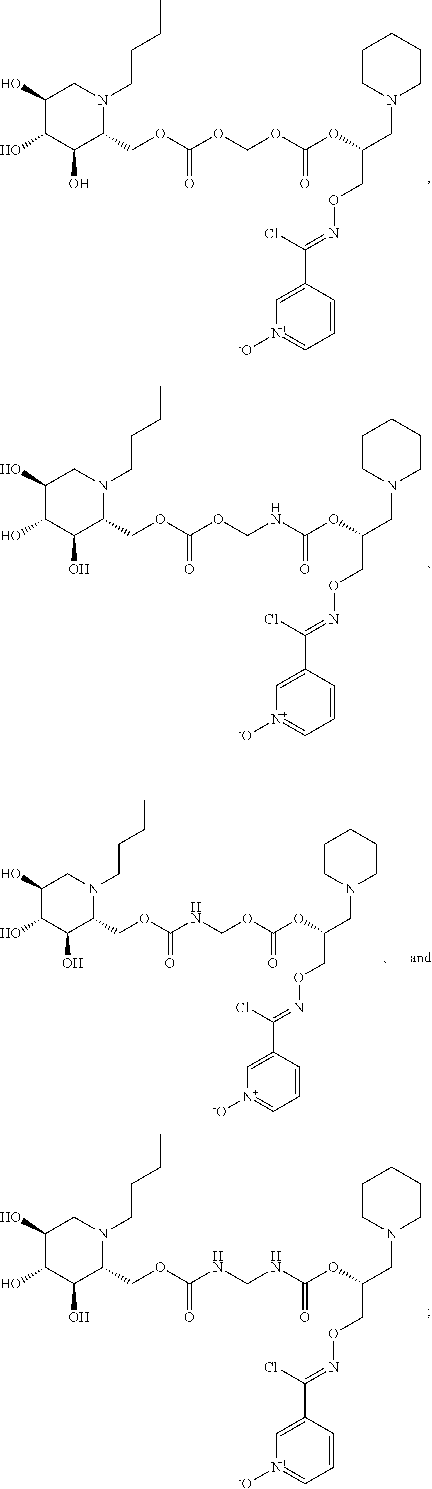

or a pharmaceutically acceptable salt thereof.

In a further embodiment, the present disclosure provides a composition comprising a therapeutically effective amount of a compound of Formula I, wherein the compound of Formula I is present in a form selected from the group consisting of a stereoisomer and a racemate, or a mixture thereof.

In a further embodiment, the present disclosure provides a composition comprising a therapeutically effective amount of a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In an embodiment, the present disclosure provides a composition comprising:
a therapeutically effective amount of a compound of Formula II:

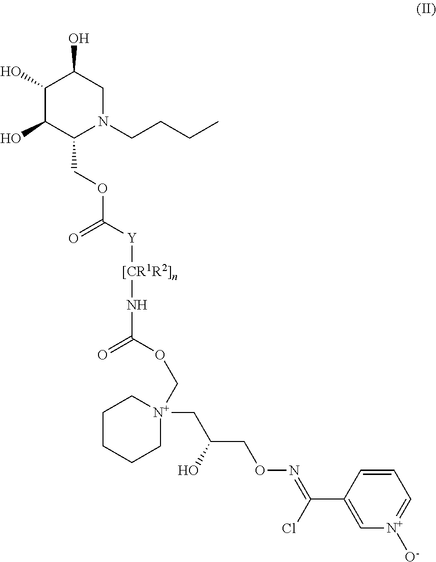

wherein Y is selected from the group consisting of O, N, or absent,
n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure describes a composition comprising a therapeutically effective amount of a compound of Formula II, wherein n is 1-3, alternatively 4-7, alternatively 8-10, alternatively 1-5, alternatively 3-7, alternatively 6-10.

In a further embodiment, the present disclosure describes a composition comprising a therapeutically effective amount of a compound of Formula II, wherein Y is absent; or a pharmaceutically acceptable salt thereof. Alternatively, the compound of Formula II is selected from the group consisting of:

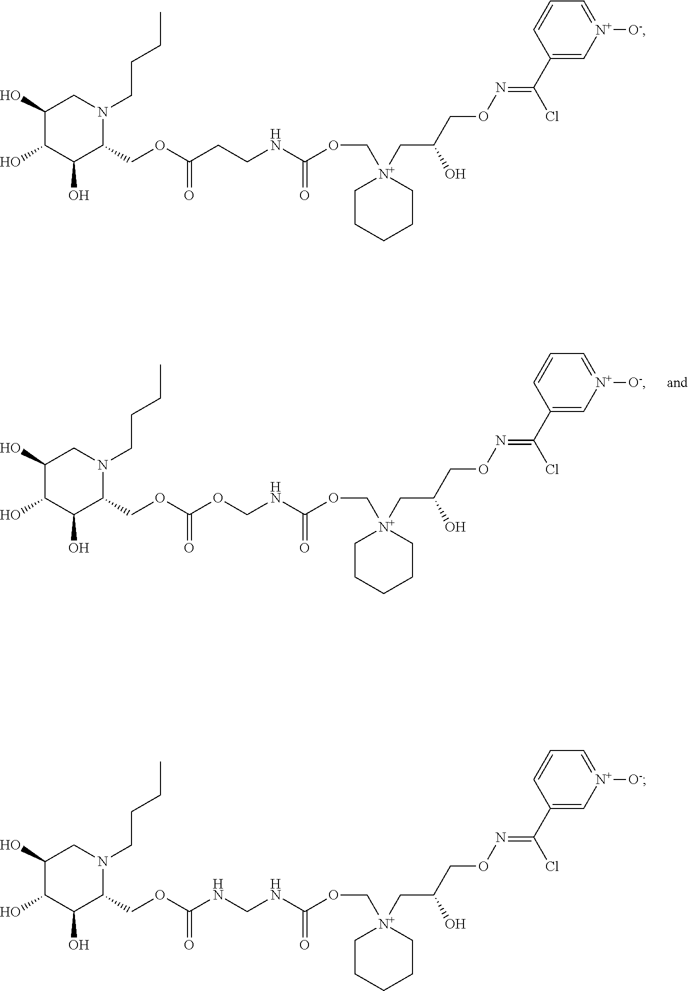

or a pharmaceutically acceptable salt thereof.

In a further embodiment, the present disclosure describes a composition comprising a therapeutically effective amount of a compound of Formula II, wherein the compound of Formula II is present in a form selected from the group consisting of a stereoisomer and a racemate, or a mixture thereof.

In a further embodiment, the present disclosure describes a composition comprising a therapeutically effective amount of a compound of Formula II, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In an embodiment, the present disclosure provides a composition comprising:
a therapeutically effective amount of a compound of Formula III:

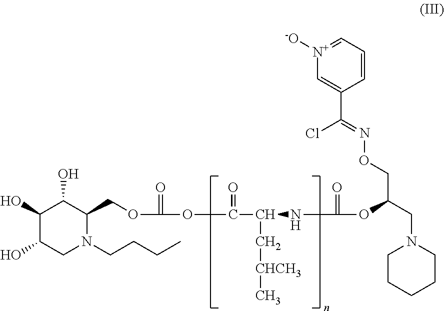

wherein n is 1-50,
or a pharmaceutically acceptable salt thereof.

In a further embodiment, the present disclosure provides a composition comprising a therapeutically effective amount of a compound of Formula III, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50

In a further embodiment, the present disclosure provides a composition comprising a therapeutically effective amount of a compound of Formula III, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In an embodiment, the present disclosure provides a composition comprising:
a therapeutically effective amount of a compound of Formula IV:

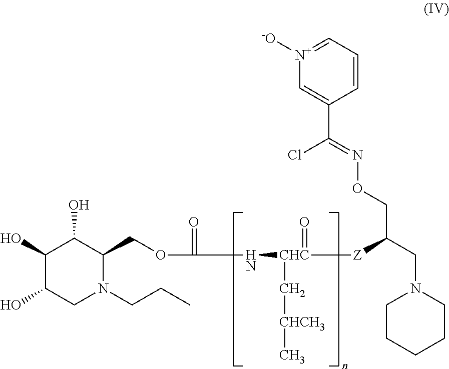

wherein Z is selected from the group consisting of O or OC(═O)O,
n is 1-50,
or a pharmaceutically acceptable salt thereof.

In a further embodiment, the present disclosure provides a composition comprising a therapeutically effective amount of a compound of Formula IV, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50

In a further embodiment, the present disclosure provides a composition comprising a therapeutically effective amount of a compound of Formula IV, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In an embodiment, the present disclosure provides a composition comprising:
a therapeutically effective amount of a compound of Formula V:

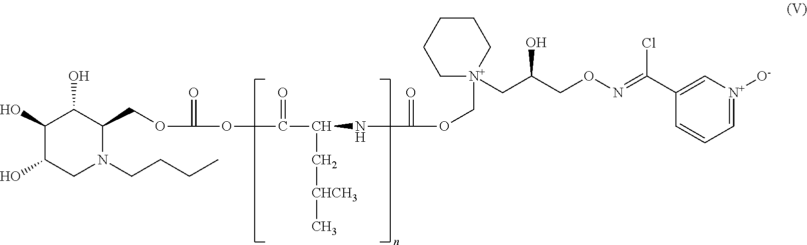

wherein n is 1-50,
or a pharmaceutically acceptable salt thereof.

In a further embodiment, the present disclosure provides a composition comprising a therapeutically effective amount of a compound of Formula V, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50

In a further embodiment, the present disclosure provides a composition comprising a therapeutically effective amount of a compound of Formula V, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further embodiment, the present disclosure provides a composition comprising a therapeutically effective amount of a compound selected from Formula I to Formula V, wherein the composition further comprises at least one excipient. In a still further embodiment, the at least one excipient is selected from the group consisting of antiadherents, binders, coatings, disintegrants, gel forming agents, fillers, flavors and colors, glidants, lubricants, preservatives, sorbents and sweeteners.

“Excipients” as it relates to the present technology means pharmaceutically inert compounds which may include one or more of the following types: antiadherents, binders, coatings, disintegrants, gel forming agents, fillers, flavors and colors, glidants, lubricants, preservatives, sorbents and sweeteners.

In a further embodiment, the present disclosure provides a composition comprising a therapeutically effective amount of a compound selected from Formula I to Formula V, wherein the composition further comprises at least one additional active pharmaceutical ingredient, wherein the at least one additional active pharmaceutical ingredient is selected from the group consisting of bimoclomol, miglustat, eliglustat, N-acetyl-L-leucine and 2-hydroxypropyl-β-cyclodextrin, arimoclomol, and combinations thereof.

In a further embodiment, the present disclosure provides a composition comprising a therapeutically effective amount of a compound selected from Formula I to Formula V, wherein the at least one additional active pharmaceutical ingredient is selected from miglustat, N-acetyl-L-leucine, and combinations thereof.

In a further embodiment, the present disclosure provides a composition comprising a therapeutically effective amount of a compound selected from Formula I to Formula V, wherein the at least one additional active pharmaceutical ingredient is a combination of miglustat and N-acetyl-L-leucine.

In a further embodiment, the present disclosure provides a composition comprising a therapeutically effective amount of a compound selected from Formula I to Formula V, wherein the composition is formulated for parenteral or intranasal administration.

“Parenteral administration” as it relates to the present technology, means administration by injection, infusion, intravenous such as through a drip line. Parenteral administration may also mean through dermal absorptions such as from a skin patch.

In a further embodiment, the present disclosure provides a composition comprising a therapeutically effective amount of a compound selected from Formula I to Formula V, wherein the composition is formulated for oral administration.

In a further embodiment, the present disclosure provides a composition comprising a therapeutically effective amount of a compound selected from Formula I to Formula V, wherein the composition is a solid oral dosage formulation include but are not limited to sublingual, gummy, chewable tablet, rapidly dissolving tablet, tablet, capsule, caplet, troche, lozenge, powder, oral thin film (OTF), oral strip, rectal film, or suppository. In some embodiments, the dosage forms are to be administered orally. Preferred oral administration forms are capsule, tablet, solutions and OTF. Solid oral dosage formulations can optionally include one or more of the following types of excipients: antiadherents, binders, coatings, disintegrants, gel forming agents, fillers, flavors and colors, glidants, lubricants, preservatives, sorbents and sweeteners.

Other compounds which may be included by admixture are, for example, medically inert ingredients, e.g., solid and liquid diluents, such as lactose, dextrose, saccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate, hydrogenated oils, sodium stearyl fumarate, and/or polyethylene glycols; gelling agents such as colloidal clays, polyethylene oxide, hydroxypropyl methyl cellulose, or carbomers; thickening agents such as gum tragacanth or sodium alginate, binding agents such as starches, arabic gums, gelatin, methylcellulose, carboxymethylcellulose or polyvinylpyrrolidone (povidone); disintegrating agents such as starch, alginic acid, alginates, crospovidone, or sodium starch glycolate; effervescing mixtures; dyestuff; sweeteners; wetting agents such as lecithin, polysorbates, poloxamer, sorbitan monoesters, glyceryl monooleates, or laurylsulfates; and other therapeutically acceptable accessory ingredients, such as humectants, preservatives, buffers and antioxidants, which are known additives for such formulations. In a still further embodiment, the solid oral dosage formulation is selected from the group consisting of a sublingual, a gummy, a chewable tablet, a rapidly dissolving tablet, a tablet, a capsule, a caplet, a troche, a lozenge, an oral powder, a solution, a thin strip, an oral thin film (OTF), an oral strip, and a rectal film.

In a further embodiment, the present disclosure provides a composition comprising a therapeutically effective amount of a compound selected from Formula I to Formula V, wherein the solid oral dosage formulation is selected from the group consisting of a sublingual, a gummy, a chewable tablet, a rapidly dissolving tablet, a tablet, a capsule, a caplet, a troche, a lozenge, an oral powder, a solution, a thin strip, an oral thin film (OTF), and an oral strip.

In a further embodiment, the present disclosure provides a composition comprising a therapeutically effective amount of a compound selected from Formula I to Formula V, wherein the composition is a liquid oral dosage formulation. In certain embodiments, the liquid oral dosage formulation is selected from the group consisting of syrups, emulsions, solutions, and suspensions.

In a further embodiment, the present disclosure provides a composition comprising a therapeutically effective amount of a compound selected from Formula I to Formula V, wherein the therapeutically effective amount of the compound of Formula I ranges from about 1 to about 5000 mg per day. In a still further aspect, the therapeutically effective amount ranges from about 50 mg to about 2500 mg, alternatively about 100 mg to about 2000 mg, alternatively about 200 mg to about 1500 mg, alternatively about 500 mg to about 1000 mg per day.

In a further embodiment, the present disclosure provides a composition comprising a therapeutically effective amount of a compound selected from Formula I to Formula V, wherein the therapeutically effective amount of the compound selected Formula I to Formula V is administered as a unit dose.

“Unit dose form” as it relates to the present technology means a single entity of a solid therapeutic dosage form (e.g., 1 capsule, 1 tablet) or a single volume dispensed from a non-solid dosage form (e.g., 5 mL of a liquid or syrup). Such a unit dose form can be from about 0.1 mg to about 5000 mg per day, alternatively from about 0.1 mg to about 300 mg per day, about 0.1 mg to about 200 mg per day, alternatively about 0.1 mg to about 100 mg per day.

In a further embodiment, the present disclosure provides a composition comprising a therapeutically effective amount of a compound selected from Formula I to Formula V, wherein the therapeutically effective amount of the compound selected from the groups Formula I to Formula V is administered in 1-2 unit doses, alternatively 1 unit dose.

In a further embodiment, the present disclosure provides a composition comprising a therapeutically effective amount of a compound selected from Formula I to Formula V, wherein the composition is an extended-release formulation, wherein the extended-release formulation further comprises a release controlling agent.

“Extended-release” as it relates to the present disclosure means one or more modifications to the pharmaceutical composition designed to release the active pharmaceutical ingredient (API) at a delayed or reduced rate compared to an unmodified pharmaceutical composition. This delayed or reduced rate of release may result in slower absorption and/or a longer elimination half-life of the API and/or its active metabolites. The delayed or reduced rate of release may be engendered through drug product formulation and/or through the prodrug metabolism.

In a further embodiment, the present disclosure provides a composition comprising a therapeutically effective amount of a compound selected from Formula I to Formula V, wherein the release controlling agent is selected from the group consisting of polymer coating, polymer matrix, and combinations thereof. In a still further embodiment, the release controlling agent is a polymer coating selected from the group consisting of polyethylene oxides (PEO), hydroxypropyl cellulose (HPC), methylcellulose (MC), hydroxypropyl methylcellulose (HPMC), hydroxyethyl cellulose (HEC), ethylcellulose (EC), and sodium carboxymethyl cellulose (CMC) In an alternative embodiment, the release controlling agent is a polymer matrix selected from the group consisting of hydroxypropylmethylcellulose (HPMC), ethylcellulose (EC), methylcellulose, hypromellose acetate succinate, hypromellose phthalate, cellulose acetate, glycerin monostearate, glyceryl monooleate, glyceryl palmitate, glyceryl behenate, hydrogenated vegetable oil, guar gum, polyvinyl alcohol, alginates, xanthan gum, carnauba wax, yellow wax, white wax, zein, carrageenan, carbomers and agar. In a still further embodiment, the release controlling agent is a combination of the polymer coating and the polymer matrix materials described above.

Methods of Treating Neurodegenerative and/or Metabolic Diseases or Disorders Using Hydroxylamine-Hydroxypiperdiene Compounds

Arimoclomol is indicated for use in combination with miglustat for the treatment of neurological manifestations of Niemann-Pick disease type C (NPC) in adult and pediatric patients 2 years of age and older.

The recommended oral dosage of arimoclomol, in combination with miglustat, for patients with an actual body weight of:
8 kg to 15 kg, is 47 mg three times a day
>15 kg to 30 kg, is 62 mg three times a day
>30 kg to 55 kg, is 93 mg three times a day
>55 kg, is 124 mg three times a day

For patients with an eGFR≥15 to <50 mL/minute, the recommended oral dosage of arimoclomol, in combination with miglustat, for patients with an actual body weight of [see Use in Specific Populations (8.6) and Clinical Pharmacology (12.3)]:
8 kg to 15 kg, is 47 mg two times a day
>15 kg to 30 kg, is 62 mg two times a day
>30 kg to 55 kg, is 93 mg two times a day
>55 kg, is 124 mg two times a day

However, for patients who have difficulty swallowing capsules, administer arimoclomol in one of two ways:

### Oral Administration

Consume the entire mixture immediately. Carefully open the capsule and sprinkle the entire contents into 15 mL of water or apple juice or 15 mL of soft food (e.g., applesauce, pudding, or yogurt).
Stir the mixture for 15 seconds.
Consume the entire mixture immediately.

Feeding Tube Administration (nasogastric or gastric tube)
Carefully open the capsule and sprinkle the entire contents into 20 mL of water. Do not add the capsule contents to other liquids besides water.
Stir the mixture for 15 seconds.
Administer the entire mixture immediately via feeding tube.
Flush the feeding tube with 5 mL of water after administration.

The safety and effectiveness of arimoclomol in combination with miglustat for the treatment of neurological manifestations of NPC have been established in pediatric patients 2 years of age and older. Use of arimoclomol in combination with miglustat for this indication is supported by evidence from a randomized, double-blind, placebo-controlled 12-month trial.

The safety and effectiveness of arimoclomol have not been established in pediatric patients younger than 2 years of age.

Arimoclomol exhibited linear and dose proportional pharmacokinetics following oral administration of doses ranging from 62 to 372 mg (three times the maximum recommended dosage) three times a day in healthy subjects. The plasma Cₘₐₓ and AUC₀₋₈ₕᵣ of arimoclomol at the 248 mg dose (two times the maximum recommended dosage) are summarized in Table X.

TABLE 1
Arimoclomol PK data

248 mg arimoclomol oral
administration three times a day

| Plasma PK Parameters | Day 1 (first dose) | Day 6 (steady state) |
|---|---|---|
| AUC_{0-8 hr} (hr · ng/mL) | 5317 (17%) | 7207 (19%) |
| Cₘₐₓ (ng/mL) | 1749 (49%) | 2090%) |

Miglustat is indicated for the treatment of certain lysosomal storage disorders in adult patients for whom enzyme replacement therapy is not a therapeutic option, by administering a 200 mg unit dosage three times a day. [Zavesca Label]

In an embodiment the present disclosure provides a method of treating a neurodegenerative or metabolic disorder in a patient in need thereof, the method comprising:
administering to the patient in need thereof a composition comprising a therapeutically effective amount of a compound of Formula I:

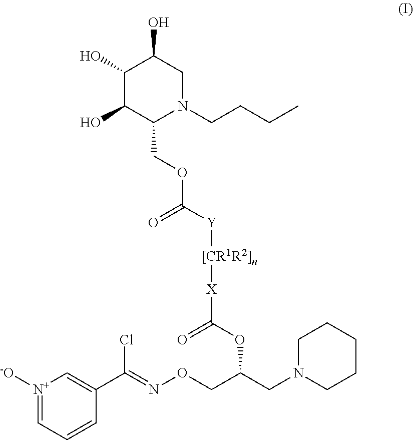

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkyl carb onyl amino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

“Neurodegenerative and or metabolic diseases or disorder” as it relates to the present disclosure means a disease or disorder wherein damage to tissue occurs due to a build up of a substance is tissue. In such diseases or disorders, build up of a substance in, for example, the neuron and or brain cells results in the progressive deterioration and death of nerve cells (neurons) in the brain and nervous system, leading to a decline in function over time. By way of another example, build up of a different substance in tissue such as the liver results in progressive deterioration and death of liver tissue, leading to a decline is function over time. Many of the neurodegenerative and/or metabolic diseases or disorders contemplated are lysosomal storage disorders.

“Lysosomal storage disorder” or “Lysosomal Storage Disorders” as it relates to the present technology means a disease or disorder associated with reduced lysosomal function. Lysosomal storage diseases (LSDs) are a group of approximately 40 rare inherited metabolic disorders that result from defects in lysosomal function. LSDs are caused by lysosomal dysfunction usually as a consequence of deficiency of a single enzyme required for the metabolism of lipids, glycoproteins or mucopolysaccharides. Although each disorder results from different gene mutations that translate into a deficiency in enzyme activity, they all share a common biochemical characteristic—all lysosomal disorders originate from an abnormal accumulation of substances inside the lysosome. Individually LSDs occur with incidences of less than 1:100.000, however, as a group the incidence is about 1:5000-1:10.000. Most of these disorders are autosomal recessively inherited, however a few are X-linked recessively inherited, such as Fabry disease. The lysosomal storage diseases are generally classified by the nature of the primary stored material involved, and can be broadly broken into the following: lipid storage disorders (or lipidoses), mainly sphingolipidoses (including Gaucher's and Niemann-Pick diseases); gangliosidosis (including Tay-Sachs disease); leukodystrophies; mucopolysaccharidoses (including Hunter syndrome and Hurler disease); glycoprotein storage disorders (glycoproteinosis); and nucolipidoses. Depending on the severity of the disease patients either die at a young and unpredictable age, many within a few months or years of birth, whereas others survive into early adulthood finally succumbing to the various pathologies of their particular disorder. The symptoms of LSD vary, depending on the particular disorder and can be mild to severe. They can include developmental delay, movement disorders, seizures, dementia, deafness and/or blindness. Some people with LSD have enlarged livers (hepatomegaly) and enlarged spleens (splenomegaly), pulmonary and cardiac problems, and abnormal bone growth. The majority of patients are initially screened by an enzyme assay, which is the most efficient method to arrive at a definitive diagnosis. In some families where the disease-causing mutation(s) is known and in certain genetic isolates, mutation analysis may be performed. As there may be numerous different mutations, sequencing of the gene encoding the particular affected enzyme is sometimes necessary to confirm the diagnosis. Prenatal diagnosis may be useful when there is a known genetic risk factor. Normal lysosomal function starts with localization to the nucleus of TFEB and TFE3, both of which activate expression of genes responsible for lysosomal protein synthesis. Lysosomes are organelles responsible for removing waste from the cell. Genes activated by TFEB and TFE3 are collectively known as the CLEAR network of genes. In cells unaffected by NPC, normally assembled and maturated proteins migrate to the lysosomal membrane where they play a critical role in transporting cholesterol and other lipids out of the lysosome. In healthy cells, this process supports elimination of waste (autophagy) and healthy neuronal function.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula I, wherein n is 1-3, alternatively 4-7, alternatively 8-10, alternatively 1-5, alternatively 3-7, alternatively 6-10

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula I, wherein X and Y are absent; or a pharmaceutically acceptable salt thereof. Alternatively, the compound of Formula I is selected from the group consisting of:

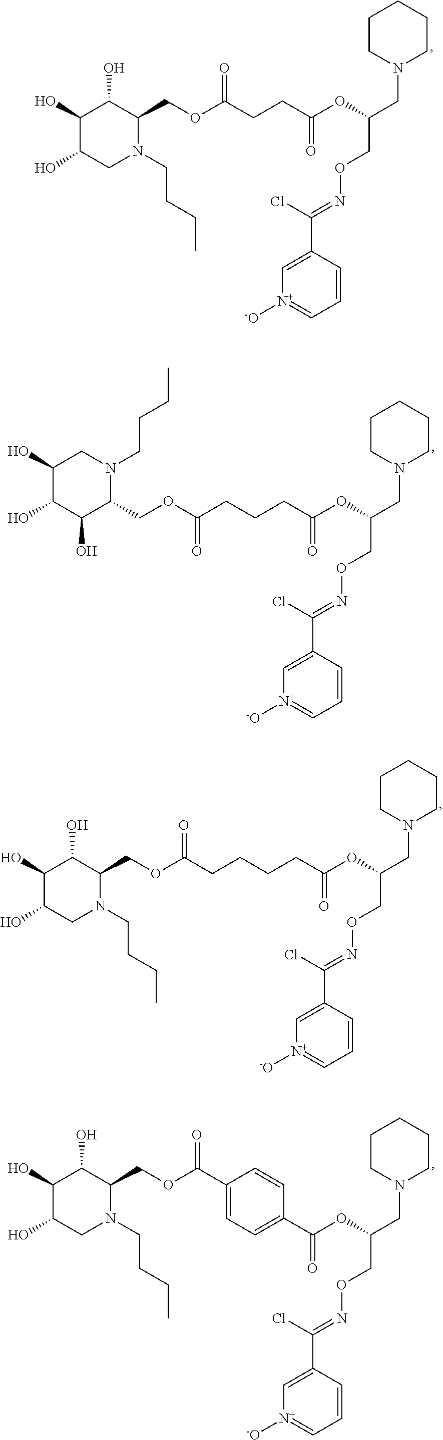

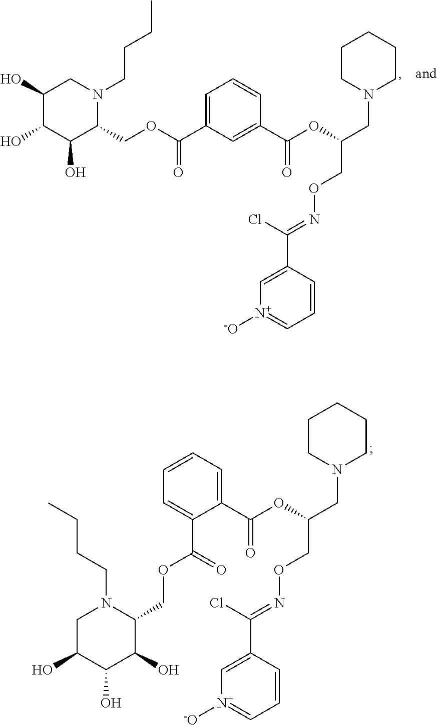

or a pharmaceutically acceptable salt thereof.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula I, wherein the compound of Formula I is present in a form selected from the group consisting of a stereoisomer and a racemate, or a mixture thereof.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula I, wherein X or Y is absent; or a pharmaceutically acceptable salt thereof. Alternatively, the compound of Formula I is selected from the group consisting of:

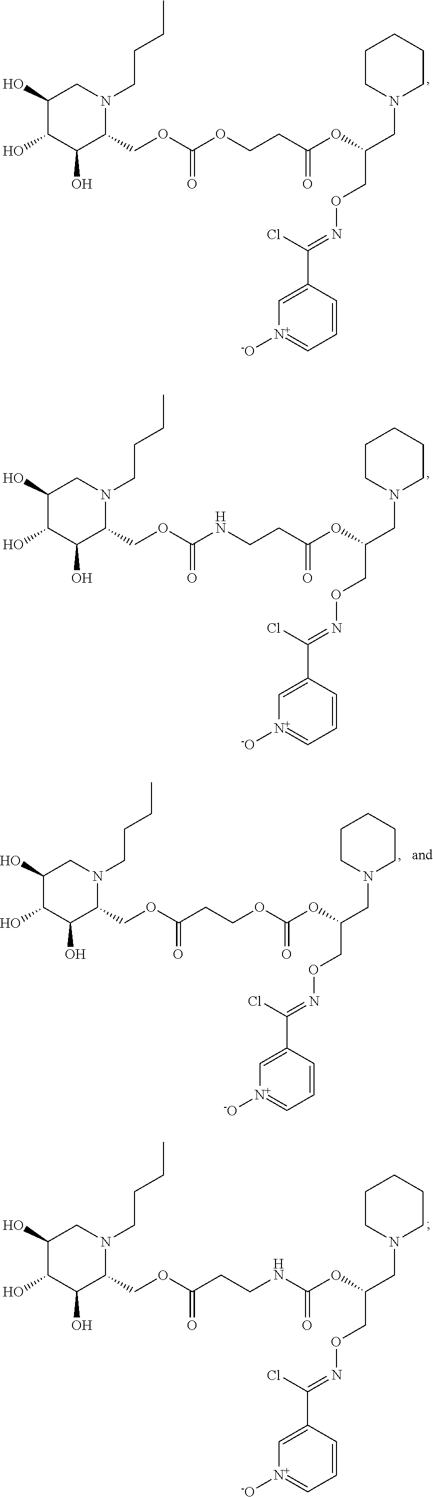

or a pharmaceutically acceptable salt thereof.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula I, wherein the compound of Formula I is present in a form selected from the group consisting of a stereoisomer and a racemate, or a mixture thereof.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula I, wherein X is N or O, and Y is N or O; or a pharmaceutically acceptable salt thereof. Alternatively, the compound of Formula I is selected from the group consisting of:

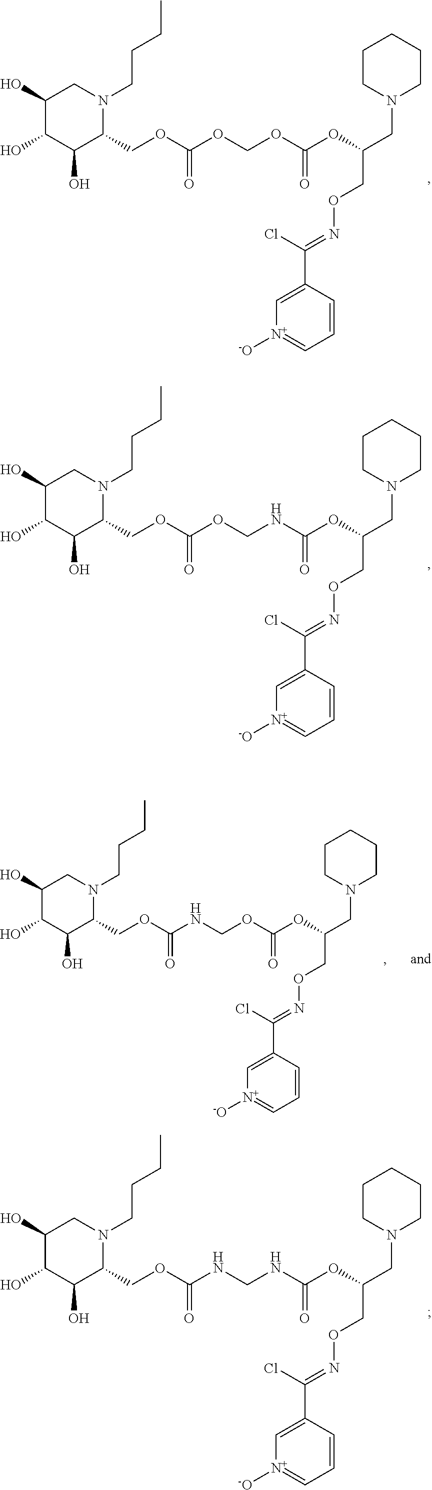

or a pharmaceutically acceptably salt thereof.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula I, wherein the compound of Formula I is present in a form selected from the group consisting of a stereoisomer and a racemate, or a mixture thereof.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula I, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In an embodiment, the present disclosure provides a method of treating a neurodegenerative or metabolic disorder in a patient in need thereof, the method comprising:
administering to the patient in need thereof a composition comprising a therapeutically effective amount of a compound of Formula II:

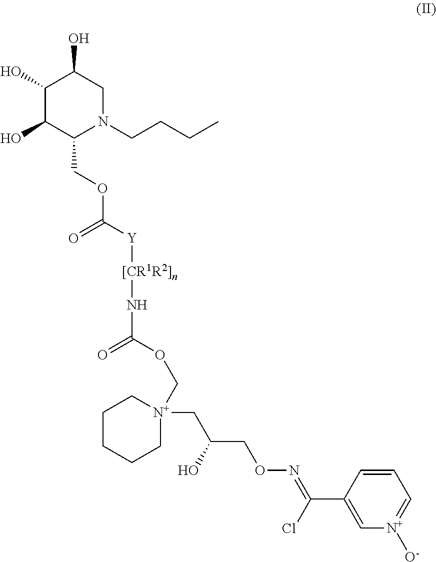

wherein Y is selected from the group consisting of O, N, or absent,
   n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula II, wherein n is 1-3, alternatively 4-7, alternatively 8-10, alternatively 1-5, alternatively 3-7, alternatively 6-10

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula II, wherein Y is absent; or a pharmaceutically acceptable salt thereof. Alternatively, the compound of Formula II is selected from the group consisting of:

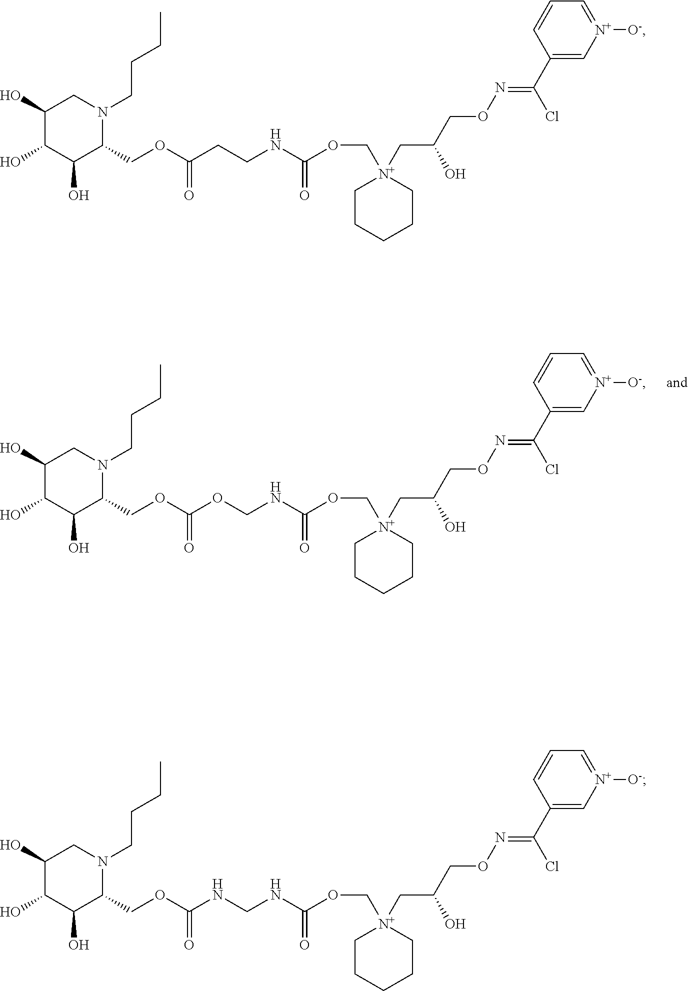

or a pharmaceutically acceptable salt thereof.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula II, wherein the compound of Formula II is present in a form selected from the group consisting of a stereoisomer and a racemate, or a mixture thereof.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula II, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further embodiment, the present disclosure provides a method of treating a neurodegenerative or metabolic disorder in a patient in need thereof, the method comprising:
administering to the patient in need thereof a therapeutically effective amount of a compound of Formula III:

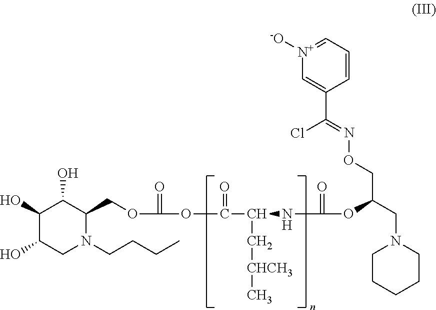

wherein n is 1-50,
or a pharmaceutically acceptable salt thereof.

In a further aspect, the present technology relates to a method of treating a neurodegenerative or metabolic disease or disorder in a patient in need thereof, wherein a method comprises administering to the patient in need thereof a composition comprising a therapeutically effective amount of the compound of Formula III, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present technology relates to a method of treating a neurodegenerative or metabolic disease or disorder in a patient in need thereof, wherein a method comprises administering to the patient in need thereof a composition comprising a therapeutically effective amount of the compound of Formula III, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further embodiment, the present disclosure provides a method of treating a neurodegenerative or metabolic disorder in a patient in need thereof, the method comprising:
administering to the patient in need thereof a therapeutically effective amount of a compound of Formula IV:

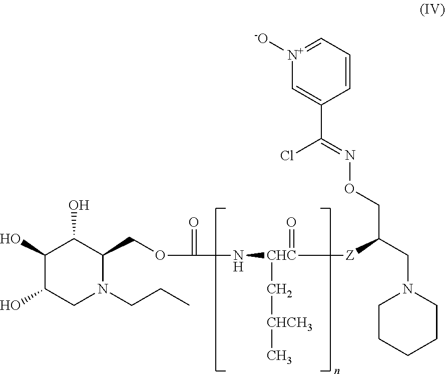

wherein Z is selected from the group consisting of O or OC(═O)O,
   n is 1-50,
or a pharmaceutically acceptable salt thereof.

In a further embodiment, the present disclosure provides a method of treating a neurodegenerative or metabolic disease or disorder in a patient in need thereof, the method comprising administering to the patient in need thereof a composition comprising a therapeutically effective amount of the compound of Formula IV, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present disclosure provides a method of treating a neurodegenerative or metabolic disease or disorder in a patient in need thereof, the method comprising administering to the patient in need thereof a composition comprising a therapeutically effective amount of the compound of Formula IV, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In an embodiment, to the present disclosure provides a method of treating a neurodegenerative or metabolic disorder in a patient in need thereof, the method comprising:
administering to the patient in need thereof a therapeutically effective amount of a compound of Formula V:

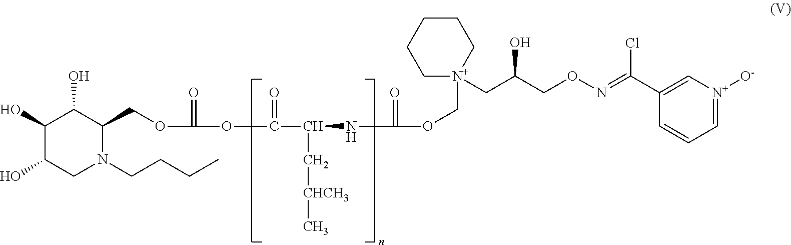

wherein n is 1-50,
or a pharmaceutically acceptable salt thereof.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula V, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present disclosure describes a method of treating a neurodegenerative or metabolic disease or disorder in a patient in need thereof, the method comprising administering to the patient in need thereof a composition comprising a therapeutically effective amount of a compound of Formula V, wherein the pharmaceutically acceptable salt is selected from the salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the therapeutically effective amount of the compound selected from the group comprising Formula I to Formula V, or mixtures thereof, ranges from about 1 to about 10000 mg per day. In a still further embodiment, the therapeutically effective amount ranges from about 50 mg to about 7500 mg, alternatively about 100 mg to about 5000 mg, alternatively about 200 mg to about 2500 mg, alternatively about 500 mg to about 1000 mg per day.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the composition is formulated for parenteral or intranasal administration.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the composition is formulated for oral or suppository administration. In a further embodiment, the composition is formulated for oral administration.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the oral formulation is a liquid oral dosage formulation. In certain embodiments, the liquid oral formulation is selected from the group consisting of syrups, emulsions, solutions, and suspensions.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the oral formulation is a solid oral dosage formulation. In certain embodiments, the solid oral dosage formulation is selected from the group consisting of a sublingual, a gummy, a chewable tablet, a rapidly dissolving tablet, a tablet, a capsule, a caplet, a troche, a lozenge, an oral powder, a solution, a thin strip, an oral thin film (OTF), and an oral strip.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the solid oral dosage is administered as a unit dosage. In certain embodiments, the unit dosage is administered in 1 to 3 unit doses. Alternatively, the unit dosage is administered in 1-2 unit doses; alternatively, the unit dosage is administered in 1 unit dose.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the neurodegenerative or metabolic disease or disorder is a lysosomal storage disorder.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the lysosomal storage disorder is selected from the group consisting of sphingolipidoses, mucopolysaccharidoses (MPS), lysosomal glycogen storage disorders, oligosaccharidoses, mucolipidoses, lipid storage disorders, glycoproteinoses, neuronal ceroid lipofuscinoses (NCL), peroxisomal biogenesis disorders (Zellweger spectrum disorders), and other lysosomal storage disorders.

:Other lysosomal storage disorders” as it relates to the present disclosure means Danon disease, cystinosis, sialic acid storage disease, Salla disease, lysosomal acid lipase deficiency (LAL-D), multiple sulfatase deficiency, pycnodysostosis, galactosialidosis, hyaluronidase deficiency (MPS IX), Hermansky-Pudlak syndrome, and pseudoxanthoma elasticum (PXE).

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the sphingolipidosis is selected from the group consisting of Gaucher disease type 1, Gaucher disease type 2, Gaucher disease type 3, Fabry disease, Niemann-pick disease type A, Niemann-pick disease type B, Niemann-pick disease type C, Tay-Sachs disease, Sandhoff disease, GM1 gangliosidosis, metachromatic leukodystrophy (MLD), Krabbe disease, and Farber lipogranulomatosis.

“Gaucher Disease” as it relates to the present technology means a disease caused by a defect in the glucosylceramidase enzyme (also known as glucocerebrosidase and acid β-glucosidase); a 55.6 KD, 497 amino acids long protein. Glucosylceramidase is responsible for the conversion of glycosylceramide (or glucocerebroside) to ceramide; the defect thus leads to an accumulation of glycosylceramide. Its activity is stimulated by BMP and is dependent on saposins. Gaucher's disease is the most common of the lysosomal storage diseases. Fatty material can collect in the spleen, liver, kidneys, lungs, brain and bone marrow. Symptoms may include enlarged spleen and liver, liver malfunction, skeletal disorders and bone lesions that may be painful, severe neurologic complications, swelling of lymph nodes and (occasionally) adjacent joints, distended abdomen, a brownish tint to the skin, anemia, low blood platelets and yellow fatty deposits on the sclera. Persons affected most seriously may also be more susceptible to infection. The disease shows autosomal recessive inheritance and therefore affects both males and females. Different mutations of glucosylceramidase determine the remaining activity of the enzyme, and, to a large extent, the phenotype. Research suggests that heterozygotes for particular glucosylceramidase mutations are at an increased risk of Parkinson's disease and particular malignancies (non-Hodgkin lymphoma, melanoma and pancreatic cancer). Glycosylceramide is a cell membrane constituent of red and white blood cells. The macrophages that clear these cells are unable to eliminate the waste product, which accumulates in fibrils, and turn into Gaucher cells, which appear on light microscopy to resemble crumpled-up paper. Gaucher's disease has three common clinical subtypes. Each type has been linked to particular mutations. In all, there are about 80 known mutations. Type I (or nonneuropathic type) Gaucher Disease is the most common form of the disease occurring in approximately 1 in 50,000 live births. It occurs most often among persons of Ashkenazi Jewish heritage, 100 times the occurrence in the general populace. Symptoms may begin early in life or in adulthood and include enlarged liver and grossly enlarged spleen (together hepatosplenomegaly); the spleen can rupture and cause additional complications. Skeletal weakness and bone disease may be extensive. Spleen enlargement and bone marrow replacement cause anemia, thrombocytopenia and leukopenia. The brain is not affected, but there may be lung and, rarely, kidney impairment. Patients in this group usually bruise easily (due to low levels of platelets) and experience fatigue due to low numbers of red blood cells. Depending on disease onset and severity, type 1 patients may live well into adulthood. Many patients have a mild form of the disease or may not show any symptoms. Type II (or acute infantile neuropathic Gaucher's disease) typically begins within 6 months of birth and has an incidence rate of approximately 1 in 100,000 live births. Symptoms include an enlarged liver and spleen, extensive and progressive brain damage, eye movement disorders, spasticity, seizures, limb rigidity, and a poor ability to suck and swallow. Affected children usually die by age 2. Type III (the chronic neuropathic Gaucher Disease) can begin at any time in childhood or even in adulthood and occurs in approximately 1 in 100,000 live births. It is characterized by slowly progressive but milder neurologic symptoms compared to the acute or type 2 version. Major symptoms include an enlarged spleen and/or liver, seizures, poor coordination, skeletal irregularities, eye movement disorders, blood disorders including anemia and respiratory problems. Patients often live into their early teen years and adulthood. The National Gaucher Foundation states that around 1 in 100 people in the general U.S. population is a carrier for type 1 Gaucher's disease, giving a prevalence of 1 in 40,000; among Ashkenazi Jews the rate of carriers is considerably higher, at roughly 1 in 15. Type 2 Gaucher's disease shows no particular preference for any ethnic group. Type 3 Gaucher's disease is especially common in the population of the Northern Swedish region of Norrbotten where the incidence of the disease is 1 in 50,000. For type 1 and most type 3 patients, enzyme replacement treatment with intravenous recombinant glucosylceramidase can decrease liver and spleen size, reduce skeletal abnormalities, and reverse other manifestations. The rarity of the disease means that dose-finding studies have been difficult to conduct, so there remains controversy over the optimal dose and dosing frequency. Due to the low incidence, this has become an orphan drug in many countries. Successful bone marrow transplantation cures the non-neurological manifestations of the disease, because it introduces a monocyte population with active glucosylceramidase. However, this procedure carries significant risk and is rarely performed in Gaucher patients. Surgery to remove the spleen (splenectomy) may be required on rare occasions if the patient is anemic or when the enlarged organ affects the patient's comfort. Blood transfusion may benefit some anemic patients. Other patients may require joint replacement surgery to improve mobility and quality of life. Other treatment options include antibiotics for infections, antiepileptics for seizures, bisphosphonates for bone lesions, and liver transplants. Substrate reduction therapy may prove to be effective in stopping Type 2, as it can cross through the blood barrier into the brain. There is currently no effective treatment for the severe brain damage that may occur in patients with types 2 and 3 Gaucher disease.

“Krabbe Disease” as it relates to the present technology means a disease caused by a defect in the β-galactosylceramidase enzyme. β-galactosylceramidase is responsible for the conversion of galactosylceramide to ceramide; the defect thus leads to an accumulation of galactosylceramide. Its activity is stimulated by BMP and is dependent on saposins. Krabbe disease is also known as globoid cell leukodystrophy or galactosylceramide lipidosis. It is a rare, often fatal degenerative autosomal recessive disorder that affects the myelin sheath of the nervous system. It occurs in about 1 in 100,000 births. A higher prevalence, about 1 in 6,000 has been reported in some Arab communities in Israel. Krabbe disease is caused by mutations in the GALC gene, which causes a deficiency of the galactosylceramidase enzyme. The lipid buildup affects the growth of the nerve's protective myelin sheath (the covering that insulates many nerves) and causes severe degeneration of motor skills. Infants with Krabbe disease are normal at birth. Symptoms begin between the ages of 3 and 6 months with irritability, fevers, limb stiffness, seizures, feeding difficulties, vomiting, and slowing of mental and motor development. In the first stages of the disease, doctors often mistake the symptoms for those of cerebral palsy. Other symptoms include muscle weakness, spasticity, deafness, optic atrophy and blindness, paralysis, and difficulty when swallowing. Prolonged weight loss may also occur. There are also juvenile- and adult-onset cases of Krabbe disease, which have similar symptoms but slower progression. In infants, the disease is generally fatal before age 2. Patients with late-onset Krabbe disease tend to have a slower progression of the disease and live significantly longer.

“Farber Disease” as it relates to the present technology means a disease associated with a defect is the acid ceramidase enzyme, which is responsible for the conversion of ceramide to sphingosine (and fatty acid); the defect thus leads to an accumulation of ceramide. Its activity is stimulated by BMP and is dependent on saponins. Acid ceramidase is also known as N-acylsphingosine amidohydrolase and is coded by the gene ASAH1. It is a heterodimeric protein consisting of a nonglycosylated alpha subunit and a glycosylated beta subunit that is cleaved to the mature enzyme post-translationally. Farber disease is also known as Farber's lipogranulomatosis, ceramidase deficiency, Fibrocytic dysmucopolysaccharidosis, and Lipogranulomatosis. It is an extremely rare autosomal recessive disease characterized by abnormalities in the joints, liver, throat, tissues and central nervous system. The liver, heart, and kidneys may also be affected. Symptoms are typically seen in the first few weeks of life and include impaired motor and mental ability and difficulty with swallowing. Other symptoms may include arthritis, swollen lymph nodes and joints, hoarseness, nodules under the skin (and sometimes in the lungs and other parts of the body), chronic shortening of muscles or tendons around joints, and vomiting. Affected persons may require the insertion of a breathing tube. In severe cases, the liver and spleen are enlarged. Currently there is no specific treatment for Farber disease. Corticosteroids can help relieve pain. Nodes can be treated with bone marrow transplants, in certain instances, or may be surgically reduced or removed. Most children with the classic form of Farber's disease die by age 2, usually from lung disease. Individuals having a milder form of the disease may live into their teenage years.

“Nieman-Pick Disease” or “NP” as it relates to the present technology means a group of rare genetic diseases of varying severity. These are inherited metabolic disorders in which sphingomyelin accumulates in lysosomes in cells of many organs. NP types A, A/B, and B are caused by mutations in the SMPD1 gene, which causes a deficiency of an acid sphingomyelinase (ASM). NP type C is now considered a separate disease, as SMPD1 is not involved, and there is no deficiency in ASM. State. Arimoclomol targets NPC etiology by both NPC1-independent and NPC1-dependent pathways: (1) via the NPC1-independent pathway, arimoclomol upregulates expression of all CLEAR genes, thereby mitigating the deleterious effects of impaired cholesterol trafficking and improving overall cell health; (2) via the NPC1-dependent pathway, CLEAR gene upregulation increases production of the NPC1 protein. Though still mutated, overproducing the reduced function protein improves lysosomal function and cholesterol elimination.

“Nieman-Pick Disease Type C” or “NPC” as it relates to the present technology means a rare progressive genetic disorder characterized by an inability of the body to transport cholesterol and other fatty substances (lipids) inside of cells. This leads to the abnormal accumulation of these substances within various tissues of the body, including brain tissue.

“Nieman-Pick Type C Protein 1” or “NPC1” as it relates to the present technology means the gene encoding the Niemann-Pick type C protein 1, also referred to in the art as the NPC intracellular cholesterol transporter 1. The term “NPC1” refers to the protein product of the NPC1 gene. In patients with NPC, the NPC1 protein has substantially diminished quantity and functionality because mutations in NPC genes prevent most NPC1 protein from completing assembly and maturation in the endoplasmic reticulum and golgi apparatus. Furthermore, the small amount of NPC1 protein that does localize in the lysosome is thought to be less effective, leading to cholesterol accumulation and neuronal death.

“NPC1^{-/-}” as it relates to the present technology means a mouse model that possesses a null allele mutation in the NPC1 gene which results in expression of a non-functional mutant NPC1 protein.

“NPC1^{nmf164}” as it relates to the present technology means a mouse model that possesses a point mutation in the NPC1 genes which results in expression of a mutant NPC1 protein with reduced function compared to wild-type NPC1 protein.

“NPC1-dependent” as it relates to the present technology means a biological pathway that involves the NPC1 gene and/or the NPC1 protein.

“NPC1-independent” as it relates to the present technology means a biological pathway that does not involve the NPC1 gene or NPC1 protein.

In certain patients with NPC, the NPC1 protein has substantially diminished quantity and functionality because mutations in NPC genes prevent most NPC1 protein from completing assembly and maturation in the endoplasmic reticulum and golgi apparatus (FIG. 12, Panel B). Furthermore, the small amount of NPC1 protein that does localize in the lysosome is thought to be less effective, leading to cholesterol accumulation and neuronal death.

“Fabry Disease” as it relates to the present technology means a disease is caused by a defect in the α-galactosidase A enzyme. α-galactosidase A is responsible for the conversion of globotriaosylceramide to lactosylceramide; the defect thus leads to an accumulation of globotriaosylceramide (also abbreviated as Gb3, GL-3, or ceramide trihexoside). Its activity is stimulated by BMP and is dependent on saposins. Fabry disease is also known as Anderson-Fabry disease, Angiokeratoma corporis diffusum, Ruiter-Pompen-Wyers syndrome, Ceramide trihexosidosis, and Sweeley-Klionsky disease. It is an X-linked recessive (inherited) disease that affects hemizygous males, as well as both heterozygous and homozygous females; males tend to experience the most severe clinical symptoms, while females vary from virtually no symptoms to those as serious as males. This variability is thought to be due to X-inactivation patterns during embryonic development of the female. Symptoms include anhidrosis (lack of sweating), fatigue, angiokeratomas (benign cutaneous injury of capillaries), burning extremity pain and ocular involvement. Angiokeratomas are tiny, painless papules that appear at any region of the body, but are predominant on the thighs, buttocks, lower abdomen, and groin. Cosmetic ocular involvement may be present showing cornea verticillata (also known as vortex keratopathy). Keratopathy may be the presenting feature in asymptomatic carriers and must be differentiated from other causes of vortex keratopathy (e.g. drug deposition in the cornea). Other ocular findings that can be seen include conjunctival aneurysms, posterior spoke-like cataracts, papilloedema, macular edema, optic atrophy and retinal vascular dilation. Kidney complications are a common and serious effect of the disease; renal insufficiency and renal failure may worsen throughout life. Proteinuria is often the first sign of kidney involvement. Cardiac complications may also occur; heart related effects worsen with age and may lead to increased risk of heart disease. Cerebrovascular effects lead to an increased risk of stroke. Other symptoms include tinnitus, vertigo, nausea, and diarrhea. Symptoms are typically first experienced in early childhood and can be very difficult to understand; the rarity of Fabry disease to many clinicians sometimes leads to misdiagnoses or ignorance. Manifestations of the disease usually increase in number and severity as an individual age.

“Tay-Sachs Disease” as it relates to the present disclosure, means a disease is caused by a genetic mutation in the HEXA gene on chromosome 15, which codes a subunit of the hexosaminidase enzyme known as hexosaminidase A. It is inherited in an autosomal recessive manner. The mutation disrupts the activity of the enzyme, which results in the build-up of the molecule GM2 ganglioside within cells, leading to toxicity. Diagnosis may be supported by measuring the blood hexosaminidase A level or genetic testing. Tay-Sachs disease is a type of GM2 gangliosidosis and sphingolipidosis. Tay-Sachs disease is caused by mutations in the HEXA gene on chromosome 15, which encodes the alpha-subunit of beta-N-acetylhexosaminidase A, a lysosomal enzyme. Hexosaminidase A is a vital hydrolytic enzyme, found in the lysosomes, that breaks down sphingolipids. When hexosaminidase A is no longer functioning properly, the lipids accumulate in the brain and interfere with normal biological processes. Hexosaminidase A specifically breaks down fatty acid derivatives called gangliosides; these are made and biodegraded rapidly in early life as the brain develops. Patients with and carriers of Tay-Sachs can be identified by a simple blood test that measures hexosaminidase A activity. In a further embodiment, the present technology describes a method of treating a neurodegenerative or metabolic disease or disorder, the method comprising administering to the patient in need thereof a therapeutically effective amount of the compound of Formula I, wherein the mucopolysaccharidosis is selected from the group consisting of MPS I, Hurler syndrome, Hurler-Scheie syndrome, Scheie syndrome, MPS II, Hunter syndrome), MPS III, Sanfilippo syndrome type A, Sanfilippo syndrome type B, Sanfilippo syndrome type C, Sanfilippo syndrome type D, MPS IV, Morquio syndrome type A, Morquio syndrome type B), MPS VI, Maroteaux-Lamy syndrome, MPS VII, Sly syndrome, MPS IX, and Natowicz syndrome.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the mucopolysaccharidosis is selected from the group consisting of MPS I, Hurler syndrome, Hurler-Scheie syndrome, Scheie syndrome, MPS II, Hunter syndrome), MPS III, Sanfilippo syndrome type A, Sanfilippo syndrome type B, Sanfilippo syndrome type C, Sanfilippo syndrome type D, MPS IV, Morquio syndrome type A, Morquio syndrome type B), MPS VI, Maroteaux-Lamy syndrome, MPS VII, Sly syndrome, MPS IX, and Natowicz syndrome.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the lysosomal glycogen storage disorder is selected from the group consisting of Pompe disease and glycogen storage disease type II.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the oligosaccharidosis is selected from the group consisting of Schindler disease, Sialidosis Type I, Sialidosis type II, aspartylglucosaminuria, alpha-mannosidosis, beta-mannosidosis, and fucosidosis.

“Sialidosis” or “Mucolipidosis type 1” as it relates to the present technology means a caused by a defect in the sialidase enzyme (or alpha-neuraminidase). Sialidase is responsible for the conversion of GM3 to lactosylceramide; the defect thus leads to an accumulation of GM3. Its activity is stimulated by BMP and is dependent on saposins. Sialidosis is inherited in an autosomal recessive manner. Symptoms are either present at birth or develop within the first year of life. In many affected infants, excessive swelling throughout the body is noted at birth. These infants are often born with coarse facial features, such as a flat nasal bridge, puffy eyelids, enlargement of the gums, and excessive tongue size (macroglossia). Many infants with are also born with skeletal malformations such as hip dislocation. Infants often develop sudden involuntary muscle contractions (called myoclonus) and have red spots in their eyes (cherry red macules). They are often unable to coordinate voluntary movement (called ataxia). Tremors, impaired vision, and seizures also occur. Tests reveal abnormal enlargement of the liver (heptomegaly) and spleen (splenomegaly) and extreme abdominal swelling. Infants generally lack muscle tone (hypotonia) and have mental retardation that is either initially or progressively severe. Many patients suffer from failure to thrive and from recurrent respiratory infections. Most infants with ML I die before the age of 1 year. Sialidosis may be sub-categorized according to the age at which symptoms begin and the types of symptoms present. The effects of the disease may range from mild to severe.

Sialidosis is a rare disorder that has no racial predilection. Very little population data are available, but a study from the Netherlands reported a frequency of approximately 1 case in 2,175,000 live births. However, this rate may not apply to all populations, some of which could have a higher incidence; moreover, missed clinical recognition is an important factor when newborn screening is not an option.

“Metachromatic Leukodystrophy (MLD)” or “Arylsulfatase A deficiency” as it relates to the present technology means a disease caused by a defect in the arylsulfatase A enzyme (or cerebroside-sulfatase). Arylsulfatase A is responsible for the conversion of sulfatide (or cerebroside 3-sulfate) to galactosylceramide; the defect thus leads to an accumulation of sulfatide. Its activity is stimulated by BMP and is dependent on saposins. It is a lysosomal storage disease which is commonly listed in the family of leukodystrophies. Leukodystrophiea affect the growth and/or development of myelin, the fatty covering which acts as an insulator around nerve fibers throughout the central and peripheral nervous systems. Like many other genetic disorders that affect lipid metabolism, there are several forms of MLD, which are late infantile, juvenile, and adult: In the late infantile form, which is the most common form MLD, affected children begin having difficulty walking after the first year of life. Symptoms include muscle wasting and weakness, muscle rigidity, developmental delays, progressive loss of vision leading to blindness, convulsions, impaired swallowing, paralysis, and dementia. Children may become comatose. Untreated, most children with this foil of MLD die by age 5, often much sooner. Children with the juvenile loan of MLD (onset between 3-10 years of age) usually begin with impaired school performance, mental deterioration, and dementia and then develop symptoms similar to the late infantile form but with slower progression. Age of death is variable, but normally within 10 to 15 years of symptom onset. The adult form commonly begins after age 16 as a psychiatric disorder or progressive dementia. Adult-onset MLD progresses more slowly than the late infantile and juvenile forms, with a protracted course of a decade or more. In rare cases the body can compensate for the deficiency and the person will exhibit no symptoms.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the mucolipidosis is selected from the group consisting of mucolipidosis II, I-cell disease, mucolipidosis III, pseudo-Hurler polydystrophy, and mucolipidosis IV.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the lipid storage disorder is selected from the group consisting of Wolman disease, cholesteryl ester storage disease, and Farber disease.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the glycoproteinosis is selected from the group consisting of alpha-mannosidosis, beta-mannosidosis, fucosidosis, aspartylglucosaminuria.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the neuronal ceroid lipofuscinosis is selected from the group consisting of infantile NCL, CLN1, CLN5, late infantile NCL, CLN2, juvenile NCL, CLN3, adult NCL, Kufs disease, CLN4, congenital NCL, and CLN10.

In a further aspect, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the peroxisomal biogenesis disorder is selected from the group consisting of Zellweger syndrome, neonatal adrenoleukodystrophy, and infantile Refsum disease.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the other lysosomal storage disorder is selected from the group consisting of Danon disease, cystinosis, sialic acid storage disease, Salla disease, lysosomal acid lipase deficiency (LAL-D), multiple sulfatase deficiency, pycnodysostosis, galactosialidosis, hyaluronidase deficiency (MPS IX), Hermansky-Pudlak syndrome, and pseudoxanthoma elasticum (PXE).

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the Niemann Pick disease Type C is Niemann Pick disease Type C 1 or Niemann Pick disease Type C 2.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the composition further comprises a therapeutically effective amount of at least one additional active pharmaceutical ingredient, wherein the at least one additional active pharmaceutical ingredient is selected from the group consisting of bimoclomol, miglustat, eliglustat, N-acetyl-L-leucine and 2-hydroxypropyl-β-cyclodextrin, arimoclomol, and combinations thereof.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the at least one additional active pharmaceutical ingredient is selected from miglustat, arimoclomol, N-acetyl-L-leucine, and combinations thereof. In a still further embodiment, the at least one additional active pharmaceutical ingredient is a combination of arimoclomol and miglustat, alternatively the at least one additional active pharmaceutical ingredient is a combination arimoclomol and N-acetyl-L-leucine, alternatively the at least one additional pharmaceutical ingredient is a combination of miglustat and N-acetyl-L-leucine.

In a further embodiment, the present disclosure describes a method of treating a neurodegenerative or metabolic disorder in a patient in need thereof, the method comprising administering to the patient in need thereof a composition comprising a therapeutically effective amount of a compound selected from Formula I to Formula V, wherein the at least one additional active pharmaceutical ingredient is a combination of miglustat, arimoclomol, and N-acetyl-L-leucine.

In an embodiment, the present disclosure describes a method of treating Niemann-Pick disease Type C in a patient in need thereof, the method comprising administering to the patient in need thereof a composition comprising a therapeutically effective amount of a compound of Formula I to Formula V, wherein the therapeutically effective amount is selected from an equivalent of 47 mg t.i.d. of arimoclomol citrate, and equivalent of 62 mg t.i.d. arimoclomol citrate, an equivalent of 93 mg t.i.d. of arimoclomol citrate. and an equivalent of 124 mg t.i.d. of arimoclomols citrate. Table 8 describes how the aforementioned equivalents are selected based on patient weight.

TABLE 2
Recommended dosing schedule for patients 2 years and older.

| | Recommended Equivalent |
|---|---|
| Patient Body Weight | of Arimoclomol base |
| 8 kg to 22 kg | 47 mg t.i.d. |
| >22-38 kg | 62 mg t.i.d. |
| >38-55 kg | 93 mg t.i.d. |
| >55 kg | 124 mg t.i.d. |

In a further embodiment, the present disclosure provides a method of treating a neurodegenerative or metabolic disease or disorder, the method comprising administering to the patient in need thereof a composition comprising a therapeutically effective amount of a compound of Formula I to Formula V, wherein the therapeutically effective amount is administered in 1 unit dose.

In a further embodiment, the present disclosure provides a method of treating a neurodegenerative or metabolic disease or disorder, the method comprising administering to the patient in need thereof a composition comprising a therapeutically effective amount of a compound of Formula I to Formula V, wherein the therapeutically effective amount is between 1 mg and 5000 mg per day. In a further embodiment, he therapeutically effective amount is between 500 mg and 2000 mg, alternatively from about 50 mg to about 2500 mg, alternatively about 100 mg to about 2000 mg, alternatively about 200 mg to about 1500 mg, alternatively about 500 mg to about 1000 mg per day.

In a further embodiment, the present disclosure provides a method of treating a neurodegenerative or metabolic disease or disorder, the method comprising administering to the patient in need thereof a composition comprising a therapeutically effective amount of a compound of Formula I to Formula V, wherein the therapeutically effective amount is administered in 1 to 2 unit doses.

In a further embodiment, the present disclosure provides a method of treating a neurodegenerative or metabolic disease or disorder, the method comprising administering to the patient in need thereof a composition comprising a therapeutically effective amount of a compound of Formula I to Formula V, wherein the therapeutically effective amount is administered in 1 unit dose.

In a further embodiment, the present disclosure provides a method of treating a neurodegenerative or metabolic disease or disorder, the method comprising administering to the patient in need thereof a composition comprising a therapeutically effective amount of a compound of Formula I to Formula V, wherein the method further comprises administering a therapeutically effective amount of at least one additional active pharmaceutical ingredient selected from the group consisting of bimoclomol, miglustat, eligustat, N-acetyl-L-leucine, arimoclomol, and 2-hydroxypropyl-β-cyclodextrin, and combinations thereof. In a still further embodiment, the at least one additional active pharmaceutical ingredient is selected from the group consisting of N-acetyl-L-leucine, 2-hydroxypropyl-β-cyclodextrin, and combinations thereof.

In an embodiment, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula V: or a pharmaceutically acceptable salt thereof, wherein the therapeutically effective amount ranges from about 0.1 mg/kg/day to about 10 mg/kg/day of arimoclomol freebase. Dosing based on patient body weight for patients under 2 years of age are listed in Table 9.

TABLE 3
Recommended dosage for patients less than 2 years of age.

| Patient Body Weight | Arimoclomol citrate dose | Arimiclomol base dose |
|---|---|---|
| 8-15 kg | 50 mg t.i.d. | 31 mg t.i.d. |
| >15-22 kg | 75 mg t.i.d. | 47 mg t.i.d. |
| >22-38 kg | 100 mg t.i.d. | 62 mg t.i.d. |
| >38-55 kg | 150 mg t.i.d. | 93 mg t.i.d. |
| >55 kg | 200 mg t.i.d | 124 mg t.i.d. |

In yet another embodiment, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the therapeutically effective amount ranges from about 1 mg/kg/day to about 8 mg/kg/day. In a embodiment, the therapeutically effective amount is 6 mg/kg/day of arimoclomol freebase.

In yet another embodiment, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the therapeutically effective amount is administered in 1 to 3 unit doses. In an embodiment, the therapeutically effective amount is administered in 3 unit doses. In an alternative embodiment, the unit dosage is 2 mg/kg/day of arimoclomol freebase.

In yet another embodiment, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the composition is liquid oral dosage formulation. In an alternative embodiment, the liquid oral dosage formulation comprises about 0.1 mg/mL to about 5 mg/mL of the compound of Formula I to Formula VII, or pharmaceutically acceptable salt thereof. In an embodiment, the liquid oral dosage formulation comprises about 3.1 mg/mL of the compound of Formula I to Formula VII, or pharmaceutically acceptable salt thereof.

In yet another embodiment, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the composition is liquid oral dosage formulation wherein the liquid oral dosage formulation made by emptying the contents of a 47 mg of arimoclomol citrate capsule into 15 mL of water. The liquid oral formulation may be administered orally or through a feeding tube. In this embodiment, the amount of the liquid oral formulation administered to the patient in need thereof is measured in 0.2 mL increments and remaining solution is discarded and not saved for later use. Alternatively the contents of a 31 mg arimoclomol citrate capsule may be emptied into 10 mL of water; alternatively, the contents of a 62 mg of arimoclomol citrate capsule may be emptied into 20 mL of water. In certain embodiments, the amount of the liquid oral formulation administered is determined according patient body weight according to Table 10.

TABLE 4
Amount of 3.1 mg/mL arimoclomol citrate liquid
formulation administered based on body weight.

| | Recommended dose (exact dose based |
|---|---|
| Patient body | on weight should be calculated |
| weight | by your doctor) |
| 5 kg | 3.2 mL 3 times a day |
| 6 kg | 3.8 mL 3 times a day |
| 7 kg | 4.4 mL 3 times a day |
| 8 kg | 5.2 mL 3 times a day |
| 9 kg | 5.8 mL 3 times a day |
| 10 kg | 6.4 mL 3 times a day |
| 11 kg | 7.0 mL 3 times a day |
| 12 kg | 7.6 mL 3 times a day |
| 13 kg | 8.4 mL 3 times a day |
| 14 kg | 9.0 mL 3 times a day |
| 15 kg | 9.6 mL 3 times a day |
| 16 kg | 10.2 mL 3 times a day |
| 17 kg | 10.8 mL 3 times a day |

ent in need thereof, the method comprising administering to the patient in need thereof a composition comprising a therapeutically effective amount of a compound selected from Formula I to Formula V, wherein the patient in need thereof is two years of age or less.

### EXAMPLES

The presently described technology and its advantages will be better understood by reference to the following examples. These examples are provided to describe specific embodiments of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### Treatment of Niemann-Pick Disease, Type C, in Patients Under 2 Years of Age Study Design

Data regarding neonatal presentation of NPC is very limited. Although most patients present during childhood with one or more neurological manifestations, very early-onset patients are usually diagnosed based on isolated systemic manifestations [3]. NPC is a very rare disease and children from newborn to 2 years of age are considered particularly fragile and vulnerable. All patients in this paediatric substudy were to receive active treatment with arimoclomol as it was not considered ethical to expose these children to placebo.

Based on feasibility, 3-5 patients were expected to be enrolled during the recruitment period preferably at the open sites participating in the main study. The very low number of patients diagnosed in the neonatal and infantile period was confirmed in a paper where a medical center reported 10 patients with neonatal onset over a period of 22 years [3].

The selection criteria for this paediatric substudy were chosen to exclude newborns (less than 6 months of age) and children with perinatal diseases other than NPC which can cause severe development failure and to avoid interference with the assessment of safety of arimoclomol during the trial.

In addition to the scheduled study visits in the paediatric substudy, the investigator was encouraged to arrange for the patient to attend the center for unscheduled visits, performing the relevant assessments. These visits could be arranged at the discretion of the investigator at any point during the paediatric substudy, to closely follow and monitor the safety of the patients.

Arimoclomol dosing was based on the patient's actual weight at a given visit to determine a dose that was as accurate as possible and accounted for the expected weight gain during the conduct of the paediatric substudy. PK sampling was to take place to analyze AUC and confirm when the target AUC had been attained.

### Dosing Regimen and Preparation

Based on population PK simulations, a starting dose of 3.2 mg arimoclomol citrate/kg t.i.d. was selected for all patients 6 to less than 24 months of age.

One 100 mg capsule was to be opened per administration, and the full content of the capsule to be dispersed into 20 mL of water resulting in a suspension/solution with a concentration of 5 mg/mL. It was also optional to disperse the IMP suspension/solution in other liquids, such as apple juice, and soft foodstuff, e.g., yogurt or apple sauce. In the dispersed state, arimoclomol could also be administered via a gastric tube (if applicable). For full administration of the dose, the tube should be flushed with 5 mL of water. Arimoclomol was to be administered orally using a 10 mL syringe with 0.2 mL graduations. The appropriate amount was withdrawn according to the formula below and the calculated number of mLs was rounded down to nearest 0.2 mL. Arimoclomol was to be administered orally t.i.d. The patient's dose in mL is based on the patient's body weight in kg (measured with 1 decimal) and calculated using the equation:

Weight (kg)×3.2 mg kg/5 mg ml=mL of solution to be withdrawn (rounded down to nearest 0.2 mL), administered t.i.d.

The initial dose was based on 3.2 mg arimoclomol citrate/kg and the patient's body weight in kg. If the initial PK sample demonstrated that a dosing adjustment was needed, the adjusted dose should be applied (instead of 3.2 mg/kg) in the above equation going forward. If PK sampling needed to be repeated, for any reason, an unscheduled visit was to be performed.

Once an enrolled patient turned 24 months of age, the dosing of arimoclomol was to be based on Table 1 (similar to dosing in the main study). This change in dose had to occur as soon as possible but no later than the patient's next clinic visit. The patient's weight was measured at each visit and the IMP dose was adjusted as required. If required, arimoclomol could be dispersed in 10-20 mL (i.e., 1-2 tablespoons) liquid (water, apple juice) or soft foodstuff (yoghurt or apple sauce) or administered via a gastric tube. If 100 mg capsules in solution were still used after a patient turned 24 months of age, the amount of mL solution to administer t.i.d. is shown in Table 3:

TABLE 5
Example of Arimoclomol Dosing Schedule
According to Patient Bodyweight

| | | Corresponding | Solution of 100 mg |
|---|---|---|---|
| Patient | | dose of | arimoclomol citrate |
| body | Arimoclomol | arimoclomol | capsule in 20 mL |
| weight | citrate dose | base^{a} | liquid (5 mg/mL) |
| 8-15 kg | 50 mg t.i.d. | 31 mg t.i.d. | 10 mL t.i.d. |
| | (150 mg/day) | | |
| >15-22 kg | 75 mg t.i.d. | 47 mg t.i.d. | 15 mL t.i.d. |
| | (225 mg/day) | | |
| >22-38 kg | 100 mg t.i.d. | 62 mg t.i.d. | 20 mL t.i.d. |
| | (300 mg/day) | | |
| >38-55 kg | 150 mg t.i.d. | 39 mg t.i.d. | 30 mL^{b} t.i.d. |
| | (450 mg/day) | | |
| >55 kg | 200 mg t.i.d. | 124 mg t.i.d. | 40 mL^{b} t.i.d. |
| | (600 mg/day) | | |

t.i.d. = 3 time per day.
^{a}Conversion factor from arimoclomol citrate to arimoclomol free base: [citrate dose] * 0.620241.
^{b}Two 100 mg arimoclomol citrate capsules are dissolved in 40 mL liquid (5 mg/mL).

If a patient who was receiving 150 mg IMP per day (50 mg t.i.d.) required a dose reduction, a dose of 75 mg per day (25 mg t.i.d) was to be administered. This corresponds to 5 mL solution of 100 mg arimoclomol citrate capsule in 20 mL liquid (5 mg/mL).

### PK Assessments

AUC_{0-8,SS} was determined from PK samples at Visit 2 to assess if the exposure level was acceptable and to describe PK in patients aged 6 to <24 months at study enrolment.

Additional PK data collected at Visits 6 and 8 were to be incorporated into a population PK model. PK samples were collected through venipuncture. At Visit 2, PK sampling to confirm dose selection was performed at 2 time-points within the time window of 6-8 hours following the first dose of arimoclomol with a minimum 0.25 hours between the 2 samples. PK sampling had to be performed prior to the second dose of arimoclomol. At Visits 6 and 8, PK samples for population PK were taken 5 min (±5 min) prior to the first daily dose of arimoclomol and 30 min (±5 min) following arimoclomol dosing. The results are presented in Table 2:

TABLE 6
PK data obtained from patients under 2 years of age.

| Patient | | Age | Bodyweight | Dose | CL | K_{TR} | K_{A} | AUC_{(0-8 h, ss)}^{a} | C_{max, ss}^{a} | C_{min, ss}^{a} |
|---|---|---|---|---|---|---|---|---|---|---|
| No. | Visit | (years) | (kg) | (μg) | (L/h) | (1/h) | (1/h) | (h · μg/L) | (μg/L) | (μg/L) |
| 1 | 2 | 2.019 | 11.8 | 31,011.6 | 18.111 | 19.454 | 0.455 | 1,712.3 | 374.8 | 118.8 |
| | 8 | 3.038 | 13.3 | 31,011.6 | 19.473 | 19.454 | 0.458 | 1,592.5 | 357.4 | 106.5 |
| 2 | 2 | 1.212 | 7.65 | 15,183.28 | 8.357 | 20.8 | 0.339 | 1,816.9 | 294.2 | 173.3 |
| | 4 (+1) | 1.288 | 7.80 | 15,183.28 | 8.471 | 20.8 | 0.34 | 1,792.3 | 291.1 | 170.4 |
| | 4 (+2) | 1.327 | 7.75 | 15,183.28 | 8.461 | 20.8 | 0.34 | 1,794.4 | 291.4 | 170.6 |
| | 5 (+1) | 1.500 | 7.50 | 15,381.75 | 8.393 | 20.8 | 0.34 | 1,832.7 | 297.2 | 174.5 |
| | 6 | 1.731 | 8.30 | 20,219.56 | 8.893 | 20.8 | 0.341 | 2,273.7 | 373.1 | 213.6 |
| | 8 | 2.192 | 9.20 | 31,011.6 | 14.649 | 20.8 | 0.342 | 2,117.0 | 387.5 | 173.5 |
| 3 | 2 | 1.731 | 9.7 | 19,227.19 | 12.862 | 17.783 | 0.331 | 1,494.9 | 261.5 | 129.2 |
| | 6 | 2.212 | 11.1 | 31,011.6 | 21.525 | 17.783 | 0.332 | 1,440.7 | 283.8 | 105.8 |
| | 8 | 2.654 | 12.0 | 31,011.6 | 22.5 | 17.783 | 0.333 | 1,378.3 | 274.7 | 99.6 |
| 4 | 2 | 1.404 | 9.97 | 19,227.19 | 6.758 | 24.073 | 0.299 | 2,844.8 | 431.5 | 288.4 |
| | 6 | 1.904 | 10.5 | 21,087.89 | 7.057 | 24.073 | 0.3 | 2,988 | 456.7 | 300.4 |
| | 8 | 2.385 | 11.0 | 31,011.6 | 11.282 | 24.073 | 0.301 | 2,748.8 | 457 | 249.6 |
| 5 | 2 | 1.615 | 10.3 | 19,847.42 | 9.081 | 17.204 | 0.289 | 2,185.5 | 343.5 | 212.1 |

^{a}Simulated values have units referring to arimoclomol freebase.
CL = clearance; K_{TR} = transit compartment rate constant; K_{A} = absorption rate constant; AUC_{(0-8 h), ss} = area under the plasma concentration curve from time 0 to 8 hours at steady state; C_{max, ss} = maximum plasma concentration at steady state; C_{min, ss} = minimum plasma concentration at steady state.

The population PK model's ability to predict arimoclomol concentrations for each patient was assessed with a range visual predictive check (VPC) which compared the observed data with simulated concentration-time profiles for 1000 virtual patients of equal age and weight profiles for the study duration. For 3 of the patients (Patient No. 2, 4, and 5) simulations from the model were consistent with observed data. For the 2 remaining patients (Patient No. 1 and 3) the observations were below the lower limit of the 90% prediction interval at visit 2, whereas improved consistency between predictions and observations were seen at later visits.

### Bayley III Scale Scoring

The Bailey III score, a three-part scale for assessing the developmental delay of children, was used to assess the development of the patients. It was implemented as it targets children between the age of 1 months and 42 months.

The Bayley III score was assessed by means of the Bayley Scales of Infant Development—Third Edition (BSID-III). This child development assessment tool was used for the following reasons: universality of use; availability of age-equivalent scores for severely impaired children; nonverbal content as on the cognitive scale. Also, the BSID-III is deemed valid as test instrument in children from cultures in western countries. The BSID-III assesses developmental functioning in infants and young children aged 1-42 months. The test consists of scores in 5 domains of function in infants: cognitive, communication, physical, social/emotional, and adaptive behavior. The following domains could be administered independently: cognitive, language, and motor scales as sub scores. The total score as well as these sub-scores were recorded separately. The BSID-III assessment was also applicable for pediatric substudy patients older than 42 months. This was justified by significant developmental delay in the patient population including all domains monitored by the scoring tool. The BSID-III was only to be administered by trained professionals who had experience and training in the test. As the test situation could be a burden for the patient it was recommended to start with the motor and cognitive scales and only continue with language if possible. The scales for social/emotional and adaptive behavior were conducted with parent questionnaires.

### Patient No. 1

With scaled scores, the patient appeared to be below average development compared to age expectations with regards to the cognitive domain and the expressive communication, receptive communication, fine motor and gross motor subscales at baseline and declined further for all but the gross motor subscale during the study. The change in GSVs indicated that the patient was largely stable in the cognitive domain, expressive communication, fine motor and gross motor subscales from baseline to visit 6, after which decline in all domains/subscales were seen through visit 9. However, for receptive communication the pattern varied more with an initial decline at visit 6, then a return to baseline level at visit 8 after which a decline was recorded at visit 9.

Overall, the patient declined in functioning over the course of study participation.

### Patient No. 2

This patient appeared to be below average development compared to age expectations with regards to the cognitive domain and the expressive communication, receptive communication, and gross motor subscales at baseline. For the fine motor subscale, the patient started out within age expectations at baseline but declined subsequently. The change in GSVs indicated that the patient was largely stable in all domains and subscales assessed from baseline to visit 11, and even with slight improvements in expressive and receptive communication from visit 9 and 10, respectively. Substantial and marked decline were seen for all domain and subscales, except receptive communication, between visits 11 and 12. However, notes at visit 12 indicate that the patient was tired and did not participate fully in the assessment, suggesting that this decline in scores was likely an artifact rather than a meaningful regression. The patient generally appeared to have stable developmental function over the course of study participation.

### Patient No. 3

At baseline, scaled scores for this patient indicated broadly age-appropriate developmental function for all domain and subscales assessed. For the gross motor subscale, a mild and gradual decrease in scaled scores falling below age expectations at visits 8 through 10 was seen. However, changes in GSVs indicated consistent improvements for all domains and subscales during the substudy compared to baseline levels. Overall, the patient appeared to gain developmental skills over the course of the substudy.

### Patient No. 4

Baseline scaled scores indicated age-appropriate abilities for the cognitive domain, receptive communication and fine motor subscales, and underdeveloped abilities compared to age expectations for the expressive communication and gross motor subscales. Data were only available from 2 visits (visit 2 and 8), however, changes in GSVs indicated modest improvement in the cognitive domain, and marked improvements in expressive communication and gross motor subscales from visit 2 to visit 8, while fine motor suggested stable abilities and receptive communication abilities declined from visit 2 to visit 8. Although limited data are available, the patient appeared with largely stable and potentially improved developmental function over the course of the substudy.

### Patient No. 5

Only baseline (visit 2) Bayley III scores were available for this patient, thus GSVs could not be calculated. Scaled scores for expressive communication, receptive communication, fine motor and gross motor subscales suggested average abilities compared to age expectations at baseline. Scaled scores for the cognitive domain suggested underdeveloped functioning compared to age expectations. Overall, the patient appeared to have broadly age-appropriate developmental function at baseline.

In summary, the evaluation of Bayley III scores indicated that 2 of the patients (Patient No. 3 and 5) were generally within the age-appropriate development window at baseline whereas 2 of the patients were generally underdeveloped compared to age expectations at baseline (Patient No. 1 and 2). The remaining patient (Patient No. 4) was age appropriate with regards to the cognitive domain, receptive communication, and fine motor subscales, and underdeveloped in the gross motor and expressive communication subscales at baseline. During the pediatric substudy, 1 patient gained developmental skills (Patient No. 3), 2 patients were largely stable over the course of the substudy (Patient No. 2 and 4), 1 patient generally declined over the course of the substudy (Patient No. 1), and 1 patient only had data available from the baseline visit (Patient No. 5).

### 10.1 Disposition of Patients

During the recruitment period (30 Oct. 2018-24 Jan. 2024), 6 patients were screened for eligibility: 1 patient was a screening failure and 5 patients were enrolled in the pediatric substudy. 4 of the 5 patients participated in the substudy for more than 12 months. Of the 5 patients enrolled, 2 completed the substudy and 3 were withdrawn prior to the planned end of study visit. For 2 of these patients (Patient No. 1 and 4) their withdrawal from trial was due to withdrawal of consent by their LAR after 1029 and 483 days in the substudy, respectively. For the last patient (Patient No. 5), the criteria for discontinuation of arimoclomol treatment was met due to AEs, the treatment interruption exceeded 4 weeks and the patient was later withdrawn from the substudy after 128 days.

Overall, change in Bayley III scores (growth scale values) provided an indication of individual performance compared to baseline. o During the substudy, 1 patient gained developmental skills, 2 patients were largely stable, and 1 patient generally declined. Results were inconclusive for 1 patient as Bayley III was only assessed at 1 timepoint.

We claim:

In one aspect of the present disclosure is claimed a compound of Formula I: wherein X is selected from the group consisting of O, N, or absent; Y is selected from the group consisting of O, N, or absent; n is 1-10; R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol; or a pharmaceutically acceptable salt thereof. In a further aspect of the present disclosure is claimed a compound of Formula I, wherein both X and Y are absent; or a pharmaceutically acceptable salt thereof. In another aspect of the present disclosure is claimed a compound of Formula I, wherein one of X or Y is absent or a pharmaceutically acceptable salt thereof. In a further aspect of the present disclosure is claimed a compound of Formula I wherein X is N or O, and Y is N or O; or a pharmaceutically acceptable salt thereof. In yet still another aspect of the present disclosure, the compound of Formula I is present in a form selected from the group consisting of a stereoisomer and a racemate, or a mixture thereof. In yet another aspect of the present disclosure,

the pharmaceutically acceptable salt of the compound of Formula I is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In yet another aspect of the present disclosure is claimed a composition of the compound of Formula I, or a pharmaceutically acceptable salt of the compound of Formula I, as described above.

In one aspect of the present disclosure is claimed a compound of Formula II wherein Y is selected from the group consisting of O, N, or absent; n is 1-10; R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol; or a pharmaceutically acceptable salt thereof. In a further aspect of the present disclosure is claimed a compound of Formula II, wherein Y is absent; or a pharmaceutically acceptable salt thereof. In yet still another aspect of the present disclosure, the compound of Formula II is present in a form selected from the group consisting of a stereoisomer and a racemate, or a mixture thereof. In yet another aspect of the present disclosure,

the pharmaceutically acceptable salt of the compound of Formula II is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In yet another aspect of the present disclosure is claimed a composition of the compound of Formula II, or a pharmaceutically acceptable salt of the compound of Formula II, as described above.

In one aspect of the present disclosure is claimed a compound of Formula III wherein n is 1-50, or a pharmaceutically acceptable salt thereof. In yet another aspect of the present disclosure, the pharmaceutically acceptable salt of the compound of Formula III is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In yet another aspect of the present disclosure is claimed a composition of the compound of Formula III, or a pharmaceutically acceptable salt of the compound of Formula III, as described above.

In one aspect of the present disclosure is claimed a compound of Formula IV wherein Z is selected from the group consisting of O or OC(═O)O; n is 1-50, or a pharmaceutically acceptable salt thereof. In yet another aspect of the present disclosure, the pharmaceutically acceptable salt of the compound of Formula IV is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In yet another aspect of the present disclosure is claimed a composition of the compound of Formula IV, or a pharmaceutically acceptable salt of the compound of Formula IV, as described above.

In one aspect of the present disclosure is claimed a compound of Formula V wherein n is 1-50, or a pharmaceutically acceptable salt thereof. In yet another aspect of the present disclosure, the pharmaceutically acceptable salt of the compound of Formula V is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In yet another aspect of the present disclosure is claimed a composition of the compound of Formula V, or a pharmaceutically acceptable salt of the compound of Formula V, as described above.

In yet another aspect of the present disclosure is claimed a composition of one or more compounds selected from the group consisting of Formula I, Formula II, Formula III, Formula IV and Formula V as described above, wherein the composition further comprises at least one excipient and/or at least one additional active pharmaceutical ingredient. In certain aspects of the present disclosure, the at least one excipient is selected from the group consisting of antiadherents, binders, coatings, disintegrants, gel forming agents, fillers, flavors and colors, glidants, lubricants, preservatives, sorbents and sweeteners. In certain aspects of the present disclosure, the at least one additional active pharmaceutical ingredient is selected from the group consisting of bimoclomol, miglustat, eliglustat, N-acetyl-L-leucine and 2-hydroxypropyl-β-cyclodextrin, arimoclomol, and combinations thereof. In another aspect of the present disclosure, the composition comprises at least one additional active pharmaceutical ingredient selected from the group consisting of miglustat, N-acetyl-L-leucine, and combinations thereof. In certain aspects of the present disclosure, the at least one additional active pharmaceutical ingredient is a combination of miglustat and N-acetyl-L-leucine. In

In yet another aspect of the present disclosure is claimed a method of treating a neurodegenerative or metabolic disorder in a patient in need thereof, the method comprising:

administering to the patient in need thereof a composition comprising a therapeutically effective amount of a compound of Formula I: wherein X is selected from the group consisting of O, N, or absent; Y is selected from the group consisting of O, N, or absent; n is 1-10; R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol; or a pharmaceutically acceptable salt thereof. In a further aspect of the present disclosure the method comprises a compound of Formula I, wherein both X and Y are absent; or a pharmaceutically acceptable salt thereof. In another aspect of the present disclosure, the method comprises a compound of Formula I, wherein one of X or Y is absent or a pharmaceutically acceptable salt thereof. In a further aspect of the present disclosure the method comprises a compound of Formula I wherein X is N or O, and Y is N or O; or a pharmaceutically acceptable salt thereof. In yet still another aspect of the present disclosure, the method comprises a compound of Formula I which is present in a form selected from the group consisting of a stereoisomer and a racemate, or a mixture thereof. In yet another aspect of the present disclosure, the method comprises a pharmaceutically acceptable salt of the compound of Formula I, wherein the pharmaceutically acceptable salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In yet another aspect of the present disclosure is claimed a method of treating a neurodegenerative or metabolic disorder in a patient in need thereof, the method comprising:

administering to the patient in need thereof a composition comprising a therapeutically effective amount of a compound of Formula II: wherein Y is selected from the group consisting of O, N, or absent; n is 1-10; R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol; or a pharmaceutically acceptable salt thereof. In a further aspect of the present disclosure the method comprises a compound of Formula II, wherein Y is absent; or a pharmaceutically acceptable salt thereof. In yet still another aspect of the present disclosure, the method comprises a compound of Formula II which is present in a form selected from the group consisting of a stereoisomer and a racemate, or a mixture thereof. In yet another aspect of the present disclosure, the method comprises a pharmaceutically acceptable salt of the compound of Formula II, wherein the pharmaceutically acceptable salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In yet another aspect of the present disclosure is claimed a method of treating a neurodegenerative or metabolic disorder in a patient in need thereof, the method comprising:
administering to the patient in need thereof a composition comprising a therapeutically effective amount of a compound of Formula III wherein n is 1-50,
or a pharmaceutically acceptable salt thereof. In yet still another aspect of the present disclosure, the compound of Formula III is present in a form selected from the group consisting of a stereoisomer and a racemate, or a mixture thereof. In yet another aspect of the present disclosure, the pharmaceutically acceptable salt of the compound of Formula III is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In yet another aspect of the present disclosure is claimed a method of treating a neurodegenerative or metabolic disorder in a patient in need thereof, the method comprising: administering to the patient in need thereof a composition comprising a therapeutically effective amount of a compound of Formula IV wherein Z is selected from the group consisting of O or OC(═O)O; n is 1-50, or a pharmaceutically acceptable salt thereof. In yet still another aspect of the present disclosure, the compound of Formula IV is present in a form selected from the group consisting of a stereoisomer and a racemate, or a mixture thereof. In yet another aspect of the present disclosure, the pharmaceutically acceptable salt of the compound of Formula IV is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In yet another aspect of the present disclosure is claimed a method of treating a neurodegenerative or metabolic disorder in a patient in need thereof, the method comprising:
administering to the patient in need thereof a composition comprising a therapeutically effective amount of a compound of Formula V wherein n is 1-50,
or a pharmaceutically acceptable salt thereof. In yet still another aspect of the present disclosure, the compound of Formula V is present in a form selected from the group consisting of a stereoisomer and a racemate, or a mixture thereof. In yet another aspect of the present disclosure,
the pharmaceutically acceptable salt of the compound of Formula V is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In yet a further aspect of the present disclosure is claimed a method of treating a neurodegenerative or metabolic disorder in a patient in need thereof, the method comprising:
administering to the patient in need thereof a composition comprising a therapeutically effective amount of one or more compounds selected from the group consisting of Formula I, Formula II, Formula III, Formula IV, and Formula V, or mixtures thereof, wherein the therapeutically effective amount of the compound selected from the group comprising Formula I, Formula II, Formula III, Formula IV, and Formula V, or mixtures thereof, ranges from about 1 to about 10000 mg per day. In yet a further aspect of the present disclosure, the method comprises a composition which is formulated for a specific administration selected from the group consisting of parenteral or intranasal administration, oral or suppository administration. In one aspect of the present disclosure, the composition is formulated for oral administration (oral formulation) in either a liquid or solid dosage formulation. In yet another aspect of the present disclosure, the oral formulation is a liquid oral dosage formulation, wherein the liquid oral formulation is selected from the group consisting of syrups, emulsions, solutions, and suspensions. In yet another aspect of the present disclosure, the oral formulation is a solid oral dosage formulation, wherein the solid oral dosage formulation is selected from the group consisting of a sublingual, a gummy, a chewable tablet, a rapidly dissolving tablet, a tablet, a capsule, a caplet, a troche, a lozenge, an oral powder, a solution, a thin strip, an oral thin film (OTF), and an oral strip. In yet still another aspect of the present disclosure, the solid oral dosage is administered as a unit dosage, wherein the unit dosage is administered in 1 to 3 unit doses.

Yet in another further aspect of the present disclosure, the neurodegenerative or metabolic disorder treated by the above described method is a lysosomal storage disorder, wherein the lysosomal storage disorder is selected from the group consisting of sphingolipidoses, mucopolysaccharidoses (MPS), lysosomal glycogen storage disorders, oligosaccharidoses, mucolipidoses, lipid storage disorders, glycoproteinoses, neuronal ceroid lipofuscinoses (NCL), peroxisomal biogenesis disorders (Zellweger spectrum disorders), and other lysosomal storage disorders. In yet another aspect of the present disclosure, the sphingolipidosis treated by the above described method is selected from the group consisting of Gaucher disease type 1, Gaucher disease type 2, Gaucher disease type 3, Fabry disease, Niemann-pick disease type A, Niemann-pick disease type B, Niemann-pick disease type C, Tay-Sachs disease, Sandhoff disease, GM1 gangliosidosis, metachromatic leukodystrophy (MLD), Krabbe disease, and Farber lipogranulomatosis. In yet another aspect of the present disclosure, the mucopolysaccharidosis treated by the above described method is selected from the group consisting of MPS I, Hurler syndrome, Hurler-Scheie syndrome, Scheie syndrome, MPS II, Hunter syndrome), MPS III, Sanfilippo syndrome type A, Sanfilippo syndrome type B, Sanfilippo syndrome type C, Sanfilippo syndrome type D, MPS IV, Morquio syndrome type A, Morquio syndrome type B), MPS VI, Maroteaux-Lamy syndrome, MPS VII, Sly syndrome, MPS IX, and Natowicz syndrome. In yet another aspect of the present disclosure, the lysosomal glycogen storage disorder treated by the above described method is selected from the group consisting of Pompe disease and glycogen storage disease type II. In yet another aspect of the present disclosure, the oligosaccharidosis treated by the above described method is selected from the group consisting of Schindler disease, Sialidosis Type I, Sialidosis type II, aspartylglucosaminuria, alpha-mannosidosis, beta-mannosidosis, and fucosidosis. In yet another aspect of the present disclosure, the mucolipidosis treated by the above described method is selected from the group consisting of mucolipidosis II, I-cell disease, mucolipidosis III, pseudo-Hurler polydystrophy, and mucolipidosis IV. In yet another aspect of the present disclosure, the lipid storage disorder treated by the above described method is selected from the group consisting of Wolman disease, cholesteryl ester storage disease, and Farber disease. In yet another aspect of the present disclosure, the glycoproteinosis treated by the above described method is selected from the group consisting of alpha-mannosidosis, beta-mannosidosis, fucosidosis, aspartylglucosaminuria. In yet another aspect of the present disclosure, the neuronal ceroid lipofuscinosis treated by the above described method is selected from the group consisting of infantile NCL, CLN1, CLN5, late infantile NCL, CLN2, juvenile NCL, CLN3, adult NCL, Kufs disease, CLN4, congenital NCL, and CLN10. In yet another aspect of the present disclosure, the peroxisomal bigogensis disorder treated by the above described method is selected from the group consisting of Zellweger syndrome, neonatal adrenoleukodystrophy, and infantile Refsum disease. In yet another aspect of the present disclosure, the other lysosomal storage disorder treated by the above described method is selected from the group consisting of Danon disease, cystinosis, sialic acid storage disease, Salla disease, lysosomal acid lipase deficiency (LAL-D), multiple sulfatase deficiency, pycnodysostosis, galactosialidosis, hyaluronidase deficiency (MPS IX), Hermansky-Pudlak syndrome, and pseudoxanthoma elasticum (PXE). In yet another aspect of the present disclosure, the Niemann Pick disease Type C treated by the above described method is selected from the group consisting of Niemann Pick disease Type C 1 or Niemann Pick disease Type C 2.

In an embodiment, the present disclosure provides a method of treating Niemann-Pick Disease, type C, in a patient in need thereof, the method comprising: administering to the patient in need thereof a composition comprising a therapeutically effective amount of a compound selected from the group consisting of Formula I, Formula II, Formula III, Formula IV, and Formula V, or a pharmaceutically acceptable salt thereof. In a further embodiment, the patient in need thereof is 2 years of age or less.

1. A compound of Formula I: 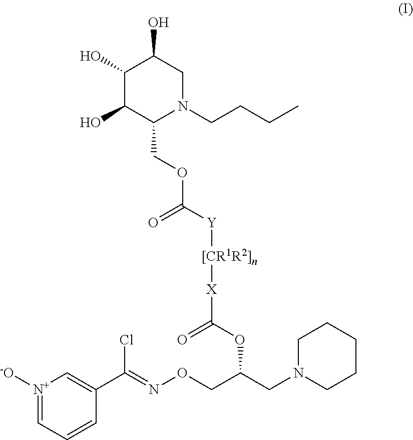
wherein X is selected from the group consisting of O, N, or absent, Y is selected from the group consisting of O, N, or absent, n is 1-10,
R¹ and R² are selected independently from each other and for each CR1R2 unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, aryl ammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

2. The compound of claim 1, wherein X and Y are absent; or a pharmaceutically acceptable salt thereof.

3. The compound of claim 1, wherein the compound of Formula I is selected from the group consisting of 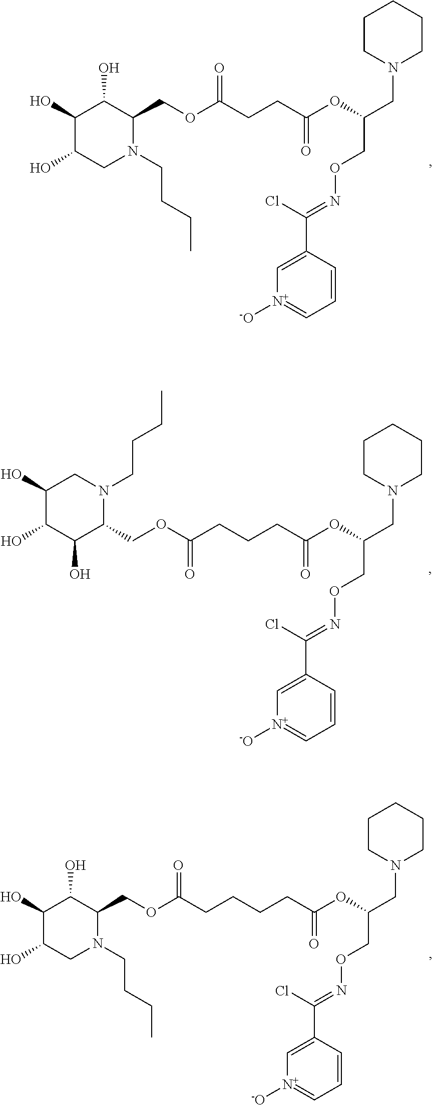 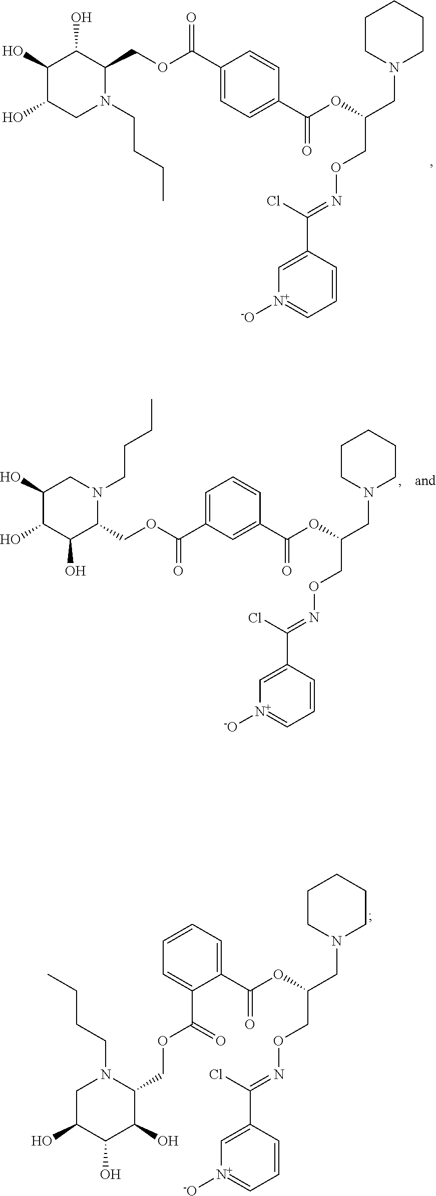
or a pharmaceutically acceptable salt thereof.

4. The compound of claim 1, wherein the compound of Formula I is present in a form selected from the group consisting of a stereoisomer and a racemate, or a mixture thereof.

5. The compound of claim 1, wherein the pharmaceutically acceptable salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

6. The compound of claim 1, wherein one of X or Y is absent; or a pharmaceutically acceptable salt thereof.

7. The compound of claim 1, wherein the compound of Formula I is selected from the group consisting of: 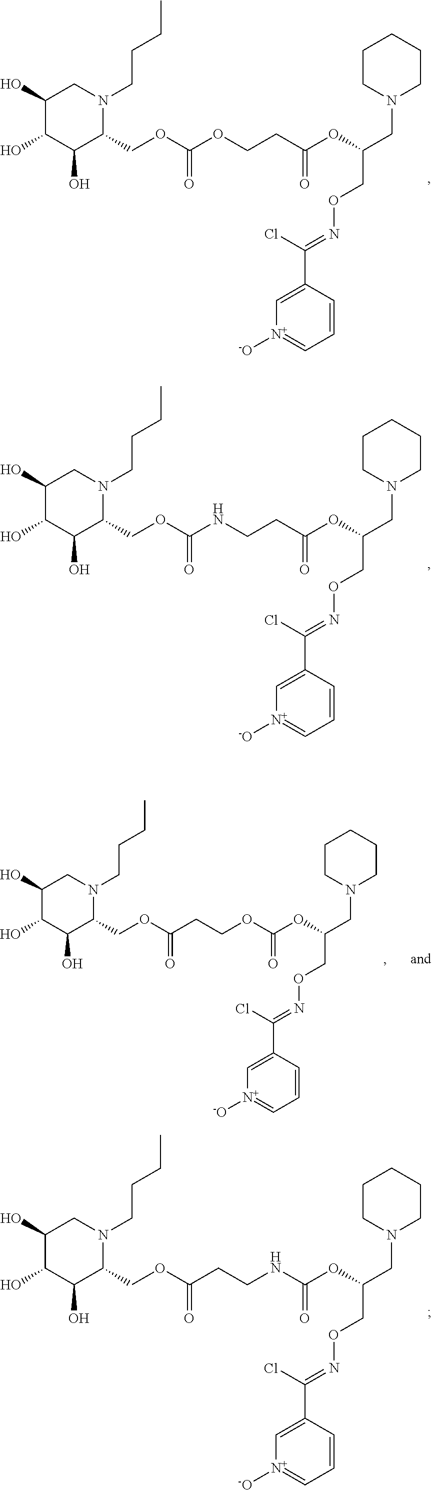
or a pharmaceutically acceptable salt thereof.

8. The compound of claim 6, wherein the compound of Formula I is present in a form selected from the group consisting of a stereoisomer and a racemate, or a mixture thereof.

9. The compound of claim 6, wherein the pharmaceutically acceptable salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

10. The compound of claim 1, wherein X is N or O, and Y is N or O; or a pharmaceutically acceptable salt thereof.

11. The compound of claim 1, wherein the compound of Formula I is selected from the group consisting of 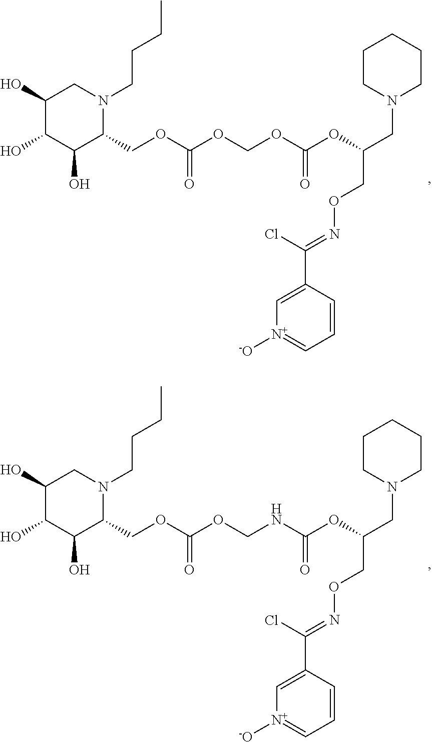
or a pharmaceutically acceptable salt thereof.

12. The compound of claim 10, wherein the compound of Formula I is present in a form selected from the group consisting of a stereoisomer and a racemate, or a mixture thereof.

13. The compound of claim 10, wherein the pharmaceutically acceptable salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

14. A compound of Formula II: 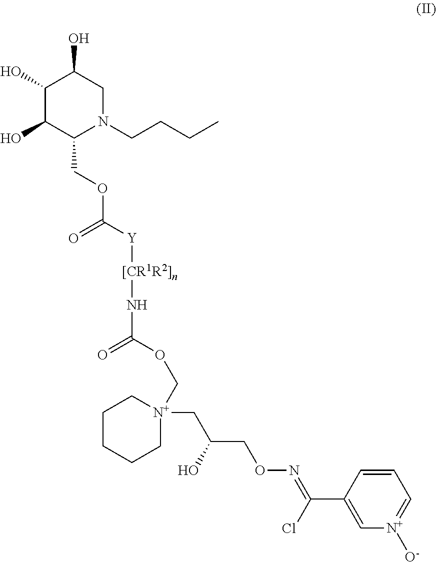
wherein Y is selected from the group consisting of O, N, or absent,
n is 1-10,
R¹ and R² are selected independently from each other and for each CR1R2 unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

15. The compound of claim 14, wherein Y is absent; or a pharmaceutically acceptable salt thereof.

16. The compound of claim 14, wherein the compound of Formula II is selected from the group consisting of 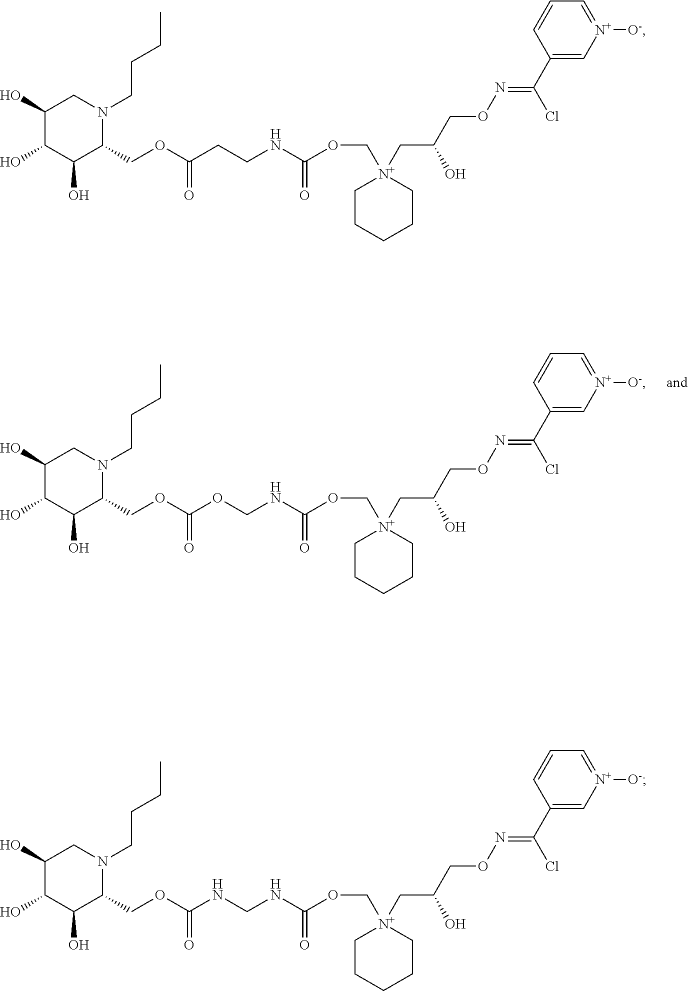
or a pharmaceutically acceptable salt thereof.

17. The compound of claim 14, wherein the compound of Formula II is present in a form selected from the group consisting of a stereoisomer and a racemate, or a mixture thereof.

18. The compound of claim 14, wherein the pharmaceutically acceptable salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

19. A compound of Formula III: 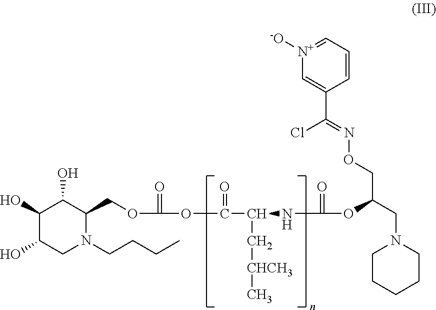
wherein n is 1-50,
or a pharmaceutically acceptable salt thereof.

20. The compound of claim 19, wherein the pharmaceutically acceptable salt is selected from the group consisting of sulfate, citrate, acetate, oxalate, hydrochloride, hydrobromide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

21.-117. (canceled)
